(19) United States
(12) Patent Application Publication
(10) Pub. No.: US 2021/0113712 A1
(43) Pub. Date: Apr. 22, 2021

(54) METHODS FOR PREPARING CELL TARGETING CONJUGATES AND CONJUGATES OBTAINABLE BY SAID METHODS

(71) Applicant: LINXIS B.V., Amsterdam (NL)

(72) Inventors: Eugen Merkul, Amsterdam (NL); Niels Jurriaan Sijbrandi, Utrecht (NL); Joey Armand Muns, Hoofddorp (NL); Augustinus Antonius Maria Silvester Van Dongen, Utrecht (NL); Paulus Johannes Gerardus Maria Steverink, Oss (NL); Hendrik Jan Houthoff, Amsterdam (NL)

(21) Appl. No.: 16/956,467

(22) PCT Filed: Dec. 19, 2018
(86) PCT No.: PCT/NL2018/050857

§ 371 Date: Jun. 19, 2020

(30) Foreign Application Priority Data
Dec. 19, 2017 (NL) 2020120

(51) Int. Cl. A61K 47/68; A61K 47/60

(52) U.S. Cl. CPC A61K 47/6889; A61K 47/6803; A61K 47/60; A61K 47/6811; A61K 47/6855

(57) ABSTRACT

Methods for preparing a cell targeting conjugate, which conjugate comprises a cell binding moiety conjugated to a secondary functional moiety. The disclosure further relates to the cell targeting conjugates obtainable by the method, to a pharmaceutical composition comprising the conjugates and to the secondary functional moieties as such. The disclosure also relates to the use of the cell targeting conjugates in the treatment of cancer.

### CROSS-REFERENCE TO RELATED APPLICATIONS

This application is a national phase entry under 35 U.S.C. § 371 of International Patent Application PCT/NL2018/050857, filed Dec. 19, 2018, designating the United States of America and published in English as International Patent Publication WO 2019/125153 A1 on Jun. 27, 2019, which claims the benefit under Article 8 of the Patent Cooperation Treaty to Dutch Patent Application Serial No. 2020120, filed Dec. 19, 2017.

### TECHNICAL FIELD

The disclosure relates to methods for preparation and characterization of cell targeting conjugates, which conjugates comprise a cell binding moiety conjugated to a functional moiety via a linker. The disclosure further relates to the cell targeting conjugates obtainable by the method and to pharmaceutical compositions comprising the conjugates. The disclosure also relates to the use of the cell targeting conjugates in the treatment of cancer.

### BACKGROUND

Cell targeting conjugates, also known as antibody-drug conjugates (ADCs), are a relatively new class of biotherapeutics that have the potency to combine the pharmacokinetics, specificity, and biodistribution of an immunoglobulin with the cell killing properties of a small-molecule drug. Delivery of drugs linked to an immunoglobulin molecule, such as antibodies, that, with preference, specifically targets a cancerous cell only, is considered a valuable tool to improve the therapeutic efficacy and to reduce the systemic toxicity of drugs used for the treatment of cancer.

Whereas non-targeted drug compounds typically reach their intended target cells via whole-body distribution and passive diffusion or receptor-mediated uptake over the cell membrane, targeted drugs home-in and concentrate mainly at the targeted tissues. Consequently, targeted drugs require smaller dosages while still allowing the drug to reach therapeutically effective levels inside the target cells and thus improving the therapeutic window. The targeting of drugs to specific cells is therefore a conceptually attractive method to enhance specificity, to decrease systemic toxicity, and to allow for the therapeutic use of compounds that are less suitable or unsuitable as systemic drugs.

Although the general concept of cell targeting conjugates is simple, their successful clinical use depends on many factors such as the choice of the immunoglobulin, of the cytotoxic drug and, importantly, of the method of linking the cytotoxic drug to the immunoglobulin since pharmacokinetics, specificity, biodistribution, and toxicity of the cell targeting conjugates can be impacted by any of these building blocks. Linkers are an essential part of antibody-drug conjugates and they account i.a. for stability in circulation, pharmacokinetics, the release of toxic drugs at the site of interest, and they may have a significant effect on the biological activity (i.a. efficacy of cell killing) of the conjugate. So, the linker can considerably affect the properties of cell targeting conjugates, and therefore it is of key importance for the efficacy and toxicity of cell targeting conjugates.

Most linking technologies make use of the covalent coupling of organic linkers to immunoglobulins via a reactive ester or a maleimide functional groups, allowing the coupling to lysine or cysteine residues of the immunoglobulin, respectively. However, it is recognized that cell targeting conjugates comprising the above mentioned covalent linker technologies are associated with a suboptimal therapeutic window.

The use of transition metal complexes has been shown to provide for a facile, elegant, and robust means to produce effective cell targeting conjugates (WO2013/103301). Due to their unique chemical features, transition metal complexes can overcome challenges often encountered in the field of cell targeting conjugates such as the absence of chemically reactive groups for conventional conjugation chemistry or the presence of unwanted chemically reactive groups on the payload. Moreover, the aggregate formation of immunoglobulins following drug conjugation readily encountered when using classical linker systems for the generation of cell targeting conjugates can be diminished.

Additionally, the modification of the immunoglobulin, e.g., the reduction of the disulfide bridges of the hinge region of the immunoglobulin in order to liberate cysteines or the introduction of cysteines by genetic engineering, as is required in most current organic linker technologies, is not required for the present method wherein transition metal complexes are used as linkers.

Using transition metal complexes to link toxic drugs to immunoglobulins renders highly stable cell targeting conjugates having pharmacokinetic properties, specificity, and biodistribution profiles similar to the native immunoglobulin. This is particularly important because only when features such as immunoreactivity of the cell binding moiety (e.g., an immunoglobulin) remains sufficiently high and its biodistribution profile remains unaltered, it will be possible to deliver the conjugated drug as a therapeutic compound to the place of interest in the body.

Whereas cell targeting conjugates have hit the “tipping point” with the recent approvals of ADCETRIS®, KADCYLA®, MYLOTARG, and BESPONSA®, these should be regarded as first-generation therapies in the field of cell targeting conjugates. At the current state of technology, in order to achieve a stable coupling of a drug to an antibody, ADCs need to be developed according to, often complex, stepwise conjugation routes for every particular clinical application. This approach is inefficient with respect to i.a. development time and the use of resources and has resulted in ADCs with limited applicability in terms of e.g., their balance between efficacy and toxicity (therapeutic window). The next wave of innovation in ADC development, therefore, requires cell targeting conjugates using a more versatile linker technology, the potential for greater efficacy, and a vast improvement of their therapeutic window. Hence, there is a clear need for a more rapid, efficient, and systematic development, characterization, and production of clinically relevant cell targeting conjugates.

### BRIEF SUMMARY

The current disclosure allows for an efficient and modular approach of ADC development and production. The disclosure foresees the use of functional moieties bound to a transition metal complex for ADC development.

A first aspect of the disclosure relates to a method for preparing a cell targeting conjugate, which conjugate comprises a cell binding moiety conjugated to a secondary functional moiety, the method comprising:
a. providing a secondary functional moiety, which secondary functional moiety comprises a transition metal complex having a primary functional moiety as a first ligand and iodide or bromide as a second ligand;
b. providing a cell binding moiety and letting the secondary functional moiety bind to the cell binding moiety via substitution of the second ligand with the cell binding moiety; and
c. treating the cell targeting conjugate of step b) with a nucleophilic agent and purifying the formed cell targeting conjugate.

A second aspect of the disclosure relates to a method for preparing a cell targeting conjugate, which conjugate comprises a cell binding moiety conjugated to a functional moiety, the method comprising:
a. providing a transition metal complex comprising a first and a second leaving ligand each chosen from iodide, bromide or chloride;
b. providing a primary functional moiety and letting the primary functional moiety bind to the transition metal complex via substitution of the first leaving ligand by the primary functional moiety, such that a secondary functional moiety is obtained comprising the primary functional moiety as a first ligand and iodide, bromide or chloride as a second ligand;
c. mixing the secondary functional moiety of step b) with an iodide or/and a bromide releasing agent, such that the second ligand of the secondary functional moiety is iodide or bromide;
d. providing a cell binding moiety and letting the secondary functional moiety bind to the cell binding moiety via substitution of the iodide or bromide of the secondary functional moiety with the cell binding moiety; and
e. treating the cell targeting conjugate of step d) with a nucleophilic agent and purifying the formed cell targeting conjugate.

It was found that for binding the secondary functional moiety to the cell binding moiety (such as an antibody) it is advantageous that the second ligand is iodide or bromide. It has been found that the use of iodide or bromide as a leaving ligand has a considerable and unexpected effect on the efficiency of conjugation of the secondary functional moiety to the cell targeting moiety and on the increased hydrolytical stability of the secondary functional moiety. Due to this increased conjugation efficiency and considering the high costs of a typical cytotoxic compound used in the ADC field, the costs of production of a cell targeting conjugate can be considerably lower.

A third aspect of the disclosure relates to cell targeting conjugates obtainable by the method according to the disclosure.

A fourth aspect of the disclosure relates to a pharmaceutical composition comprising the cell targeting conjugates.

A fifth aspect of the disclosure relates to the use of the cell targeting conjugates in the treatment of cancer.

A sixth aspect of the disclosure relates to the secondary functional moieties used in the method of the disclosure. The secondary functional moieties according to the disclosure comprise a transition metal complex, such as a platinum complex, which complex has a primary functional moiety (e.g., an unmodified or modified cytotoxic drug) as a first ligand and iodide or bromide as a second ligand. Secondary functional moieties comprising an iodide or bromide group as a second ligand show an improved binding efficiency to cell binding moieties (e.g antibodies). Furthermore, the secondary functional moieties according to the disclosure are hydrolytically more stable. Moreover, the secondary functional moieties of the disclosure having iodide or bromide as a leaving ligand are also more apolar compared to the secondary functional moieties having chloride as a leaving ligand, which allows a more efficient separation (e.g., by means of preparative HPLC) of the corresponding secondary functional moieties from the unreacted primary functional moieties, which might still be present in the reaction mixture after step b) of the second aspect of the method described above.

### BRIEF DESCRIPTION OF THE DRAWINGS

FIG. 1. Conjugation efficiencies in the presence of different halide salts.

FIG. 2. Conjugation efficiencies of different monoclonal antibodies. Note: In brackets, the biological targets were indicated.

FIG. 3. Conjugation efficiencies in the presence of iodide and bromide salts having different cations. Note: NaIO₃ was used as an iodine-containing non-iodide salt (negative control).

FIG. 4. Conjugation efficiencies at different pH values (different 20 mM buffers containing 10 mM NaI); a) pH range 3.65-10.55; b) pH range 6.52-9.45.

### DETAILED DESCRIPTION

### Definitions

The term “cell targeting conjugate” as used herein has its conventional meaning and refers to a primary functional moiety, such as a therapeutic compound, diagnostic compound, chelating agent, dye, or any model compound coupled to a cell binding moiety, such as an antibody, via a linker. Cell targeting conjugates involving antibodies are also referred to as antibody-drug conjugates. However, it is noted that within the realm of the disclosure other types of cell binding moieties other than antibodies may be used.

The term “cell binding moiety” as used herein has its conventional meaning and refers to a member of a specific binding pair, i.e., a member of a pair of molecules wherein one of the pair of molecules has an area on its surface or a cavity that specifically binds to, and is therefore defined as complementary with, a particular spatial and polar organization of the other molecule, so that the molecule pair has the property of binding specifically to each other. Examples of cell binding moieties according to the disclosure are antibodies and antibody fragments.

The term “primary functional moiety” (PFM) as used herein refers to a molecule, which has the structural ability to form a coordination bond with a transition metal complex. Typical functional moieties are therapeutic compounds (i.e., drugs) or diagnostic compounds (i.e., tracers or dyes) having or being equipped with a suitable coordination group, which is able to make a coordinative bond to the metal center such as Pt(II).

The term “secondary functional moiety” (SFM) or “semi-final product” as used herein refers to a molecule comprising a transition metal complex, such as a platinum complex, having a first ligand and a second ligand, wherein the first ligand is a “primary functional moiety” (e.g., a modified or unmodified cytotoxic drug), which was defined above, and the second ligand is iodide, bromide or chloride, preferably iodide or bromide. When allowing the secondary functional moiety to bind to a cell binding moiety, the second ligand (e.g., iodide or bromide) is substituted by the cell binding moiety. Hence, if the primary functional moiety (e.g., a modified or unmodified cytotoxic drug) and the cell binding moiety (e.g., an antibody) are bound to each other, the transition metal complex functions as a linker between them.

The term “linker” as used herein has its conventional meaning and refers to a chemical moiety, which forms a bridge-like structure between a cell binding moiety and a primary functional moiety, such that the latter two are bound to each other.

The term “ligand” as used herein has its conventional meaning and refers to an ion (such as halide) or a molecule (such as a primary functional moiety) that binds to a central metal atom or ion (such as Pt(II)) to form a coordination complex.

The term “transition metal complex” as used herein has its conventional meaning and refers to a central transition metal atom or ion, which is called the coordination center, and a surrounding array of bound molecules or ions, that are known as ligands or complexing agents. A specific example of a preferred transition metal complex used in this disclosure is a platinum(II) complex.

The term “Lx” as used herein refers to a structural fragment of a transition metal complex M(Nu₁-Nu₂) comprising a combination of a metal center with a bidentate ligand:

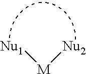

wherein M represents a metal ion or atom, which preferably is Pt(II), and Nu is a nucleophilic group wherein Nu₁ and Nu₂ can be structurally the same group or different groups and which together with the dotted line between Nu₁ and Nu₂ represent a bidentate ligand.

A first aspect of the disclosure relates to a method for preparing a cell targeting conjugate, which conjugate comprises a cell binding moiety conjugated to a secondary functional moiety, the method comprising:
a. providing a secondary functional moiety, which secondary moiety comprises a transition metal complex having a primary functional moiety as a first ligand and iodide or bromide as a second ligand;
b. providing a cell binding moiety and letting the secondary functional moiety bind to the cell binding moiety via substitution of the second ligand, which is iodide or bromide, with the cell binding moiety; and
c. treating the cell targeting conjugate of step b) with a nucleophilic agent and purifying the formed cell targeting conjugate.

It was found that for binding the secondary functional moiety to the cell binding moiety (such as an antibody) it is advantageous if the second ligand is iodide or bromide. It has been found that the use of iodide or bromide as a leaving ligand has a considerable and unexpected effect on the conjugation efficiency of the secondary functional moiety to the cell targeting moiety and on the increased hydrolytical stability of the secondary functional moiety. Due to this increased conjugation efficiency and considering the high costs of a typical cytotoxic compound used in the ADC field, the costs of production of a cell targeting conjugate can be considerably lower.

A second aspect of the disclosure relates to a method for preparing a cell targeting conjugate, which conjugate comprises a cell binding moiety conjugated to a primary functional moiety, the method comprising:
a. providing a transition metal complex comprising a first and a second leaving ligand each chosen from iodide, bromide or chloride;
b. providing a primary functional moiety and letting the functional moiety bind to the transition metal complex via substitution of the first leaving ligand by the primary functional moiety, such that a secondary functional moiety is obtained comprising the primary functional moiety as a first ligand and iodide, bromide or chloride as a second ligand;
c. mixing the secondary functional moiety of step b) with an iodide or/and a bromide releasing agent, such that the second ligand of the secondary functional moiety is iodide or bromide;
d. providing a cell binding moiety and letting the secondary functional moiety bind to the cell binding moiety via substitution of the iodide or bromide of the secondary functional moiety with the cell binding moiety; and
e. treating the cell targeting conjugate of step d) with a nucleophilic agent and purifying the formed cell targeting conjugate.

The second aspect of the present method enjoys the same advantages as the first aspect of the present method. The difference between the two aspects is that according to the second aspect of the present method a secondary functional moiety may be used, which may also comprise a chloride as a leaving ligand. However, in order to increase the conjugation efficiency, the second ligand of the secondary functional moiety, in case it is a chloride, is substituted by iodide or bromide by the addition of an iodide or a bromide releasing agent. Therefore, for the increase of the conjugation efficiency the second ligand of the secondary functional moiety can indistinguishably be iodide, bromide or chloride. All of them will yield the same product after addition of the necessary amount of an iodide or bromide releasing agent, and the efficiency of the conjugation will be considerably increased in all cases.

According to the disclosure it is possible to treat the secondary functional moiety comprising chloride as a second ligand first with an iodide or/and a bromide releasing agent, thus allowing the halide exchange, and subsequently perform the conjugation to the cell binding moiety. However, it is also possible to perform these steps simultaneously, i.e., combining steps c) and d) of the second aspect of the disclosure in a single process step. In any case the result will be that a secondary functional moiety having iodide or bromide as a second ligand will bind to the cell binding moiety with a higher efficiency than a secondary functional moiety having chloride as a second ligand in the absence of an iodide or a bromide releasing agent.

Also provided is a secondary functional moiety comprising a transition metal complex as defined in aspect having a primary functional moiety as a first ligand and iodide or bromide as a second ligand. In a preferred embodiment, first ligand is an auristatin derivative such as auristatin E and F or monomethyl auristatin E and F. Preferably auristatin F is used. Such secondary functional moiety is preferably obtainable as an intermediate product in a method according to the disclosure. The secondary functional moiety according to the disclosure comprises a transition metal complex, such as a platinum complex, which complex has a primary functional moiety (e.g., an unmodified or modified cytotoxic drug) as a first ligand and iodide or bromide as a second ligand. Secondary functional moieties comprising an iodide or bromide group as a second ligand show an improved binding efficiency to cell binding moieties (e.g., antibodies). Furthermore, the secondary functional moieties having iodide or bromide as a leaving ligand according to the disclosure are hydrolytically more stable. Moreover, the secondary functional moieties of the disclosure having iodide or bromide as a leaving ligand are also more apolar compared to the secondary functional moieties having chloride as a leaving ligand, which allows a more efficient separation (e.g., by means of preparative HPLC) of the corresponding secondary functional moieties from the unreacted primary functional moieties which might still be present in the reaction mixture after step b) of the second aspect of the method described above.

In an embodiment of the disclosure the transition metal complex of the secondary functional moiety may comprise a spacer. In such a case the primary functional moiety (e.g., an unmodified or modified cytotoxic drug) may be bound to the spacer-transition metal complex species rather than be bound directly to the metal center, which preferentially is platinum(II), of the transition metal complex. Examples of spacers are substituted or unsubstituted unbranched or branched aliphatic or heteroaliphatic chains bearing a saturated or unsaturated heterocyclic moiety, an amine or other donor group capable to bind to the metal center of the transition metal complex.

Furthermore, the secondary functional moieties are preferably provided in an isolated form and may be stored separately prior to being used in the method of the disclosure.

The secondary functional moieties comprise a transition metal complex having at least two ligands. The first ligand is a primary functional moiety and the second ligand is iodide, bromide or chloride, preferably iodide or bromide, most preferably iodide. The transition metal used is preferably platinum(II). Furthermore, the complex preferably comprises a bidentate ligand, which bidentate ligand preferably represents various substituted or unsubstituted diamine structures.

The secondary functional moiety according to the disclosure is represented by the following formula:

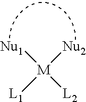

wherein L₁ or L₂ both represent ligands, wherein one of the ligands L₁ or L₂ is a leaving ligand and is chosen from iodide, bromide or chloride and the other ligand is a primary functional moiety; Nu is a nucleophilic group wherein Nu₁ and Nu₂ can be structurally the same group or different groups and which together with the dotted line between Nu₁ and Nu₂ represent a bidentate ligand; M is a transition metal atom or metal ion, preferably platinum(II).

Particular examples of bidentate ligands according to the above mentioned formula are: ethane-1,2-diamine (1), propane-1,2-diamine (2), butane-2,3-diamine (3), 2-methylpropane-1,2-diamine (4), 2,3-diaminobutane-1,4-diol (5), 2,3-diaminopropanoic acid (6), 2,3-diaminosuccinic acid (7), 3,4-diaminobutanoic acid (8), N¹,N²-dimethylethane-1,2-diamine (9), N¹-methylethane-1,2-diamine (10), N¹,N¹-dimethylethane-1,2-diamine (11), N¹,N¹,N²-trimethylethane-1,2-diamine (12), N¹,N¹,N²,N²-tetramethylethane-1,2-diamine (13), N¹,N²-diethylethane-1,2-diamine (14), N¹,N²-dipropylethane-1,2-diamine (15), N¹,N²-diisopropylethane-1,2-diamine (16), 2-((2-aminoethyl)amino)ethan-1-ol (17), 2,2′-(ethane-1,2-diylbis(azanediyl))bis(ethan-1-ol) (18), 2,2′-(ethane-1,2-diylbis(azanediyl))bis(butan-1-ol) (19), 2,2′,2″,2″-(ethane-1,2-diylbis(azanetriyl))tetrakis(ethan-1-ol) (20), 3-((2-aminoethyl)amino)propan-1-ol (21), (2-aminoethyl)glycine (22), 3-((2-aminoethyl)amino)propanoic acid (23), 2,2′-(ethane-1,2-diylbis(azanediyl))diacetic acid (24), 3,3′-(ethane-1,2-diylbis(azanediyl))dipropionic acid (25), 3-((2-aminoethyl)amino)propane-1-sulfonic acid (26), N¹-(2-aminoethyl)ethane-1,2-diamine (27), N¹-(2-aminoethyl)-N¹-methylethane-1,2-diamine (28), N¹,N¹-bis(2-aminoethyl)ethane-1,2-diamine (29), piperazine (30), decahydroquinoxaline (31), decahydroquinoxaline-6-carboxylic acid (32), (decahydroquinoxalin-6-yl)methanol (33), pyrrolidin-2-ylmethanamine (34), 1-(pyrrolidin-2-yl)ethan-1-amine (35), 2,2′-bipyrrolidine (36), piperidin-2-ylmethanamine (37), 1-(piperidin-2-yl)ethan-1-amine (38), 2,2′-bipiperidine (39), pyrrolidin-3-amine (40), 4-aminopyrrolidin-3-ol (41), pyrrolidin-3-ylmethanamine (42), cyclohexane-1,2-diamine (43), 4-methylcyclohexane-1,2-diamine (44), N¹,N²-dimethylcyclohexane-1,2-diamine (45), N¹,N¹,N²,N²-tetramethylcyclohexane-1,2-diamine (46), cyclohex-4-ene-1,2-diamine (47), (3R,4R,5S,6R)-3,4-diamino-6-(hydroxymethyl)tetrahydro-2H-pyran-2,5-diol (48), (4aR,6R,7R,8R,8aS)-6-methoxy-2-phenylhexahydropyrano[3,2-d][1,3]dioxine-7,8-diamine (49), cyclopentane-1,2-diamine (50), cyclobutane-1,2-diamine (51), cyclopropane-1,2-diamine (52), 1-benzylpyrrolidine-3,4-diamine (53). The bidentate ligands have following chemical structures:

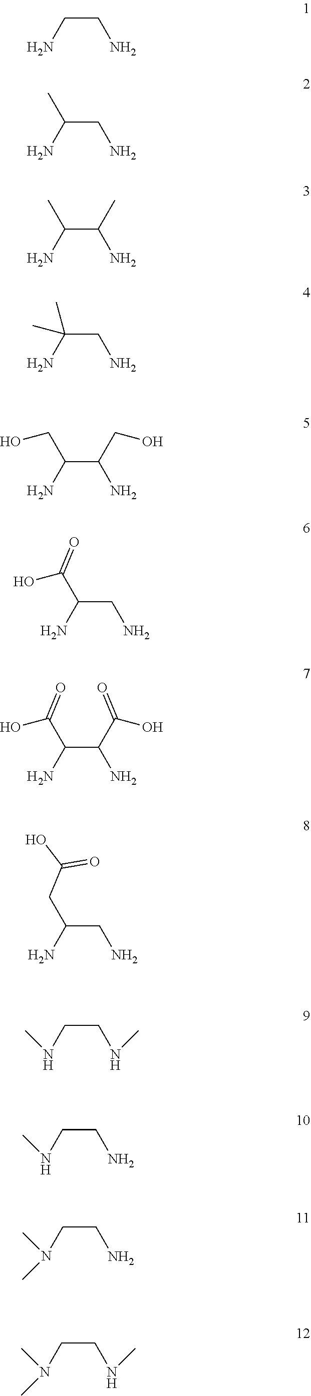

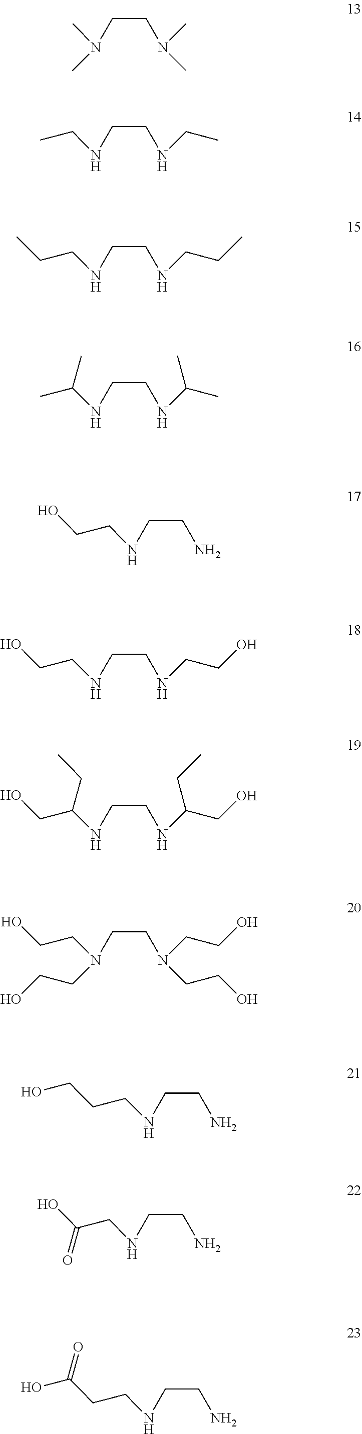

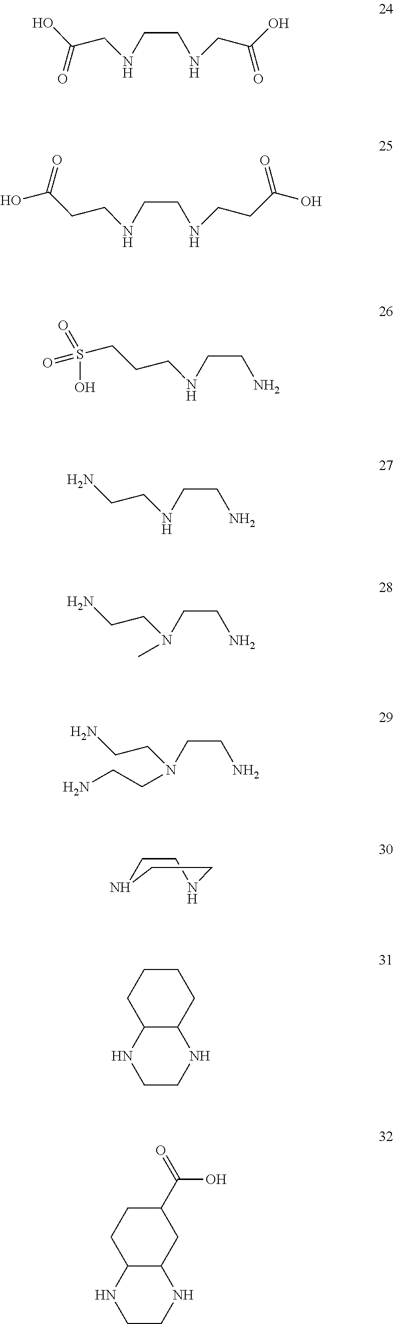

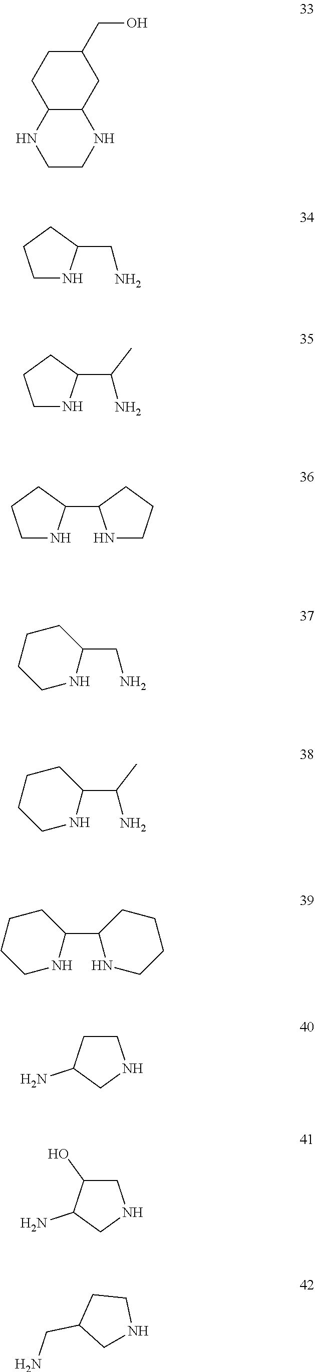

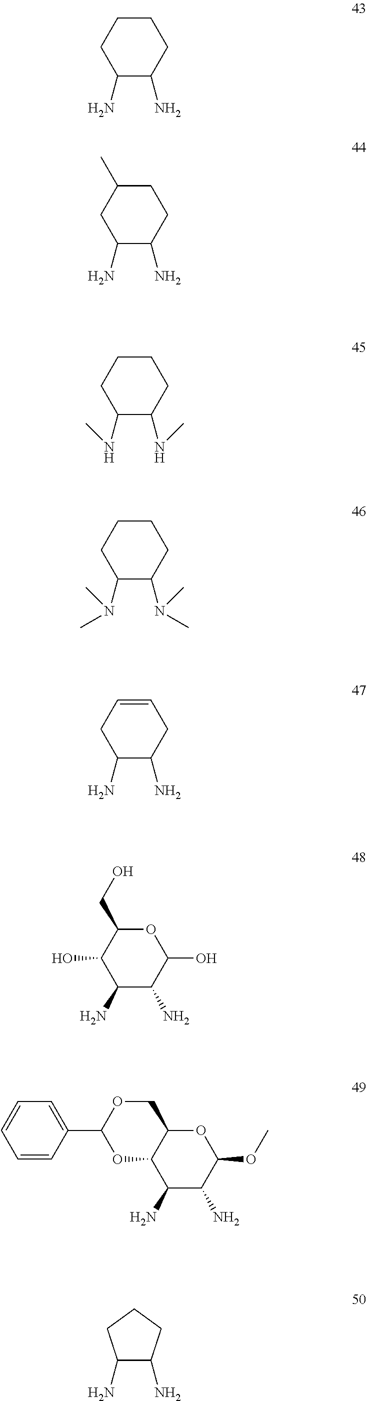

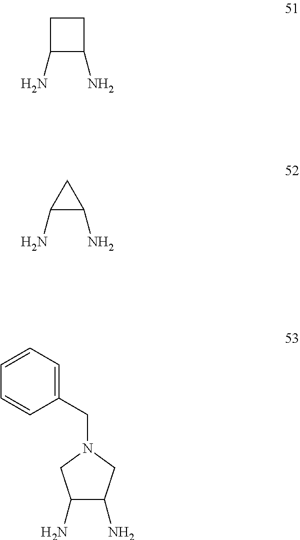 [SMILES: NC1CCC1N ; NC1CC1N ; NC1CN(Cc2ccccc2)CC1N]

Further examples of bidentate ligands according to the above mentioned formula are: propane-1,3-diamine (54), butane-1,3-diamine (55), butane-1,3-diamine (56), 2,4-diaminobutanoic acid (57), 2,4-diaminopentanedioic acid (58), 2,2-dimethylpropane-1,3-diamine (59), cyclobutane-1,1-diyldimethanamine (60), (tetrahydro-2H-pyran-4,4-diyl)dimethanamine (61), 2,2-bis(aminomethyl)propane-1,3-diol (62), cyclohexane-1,1-diyldimethanamine (63), 2-methylpropane-1,3-diamine (64), 1,3-diaminopropan-2-ol (65), 2-(aminomethyl)-2-methylpropane-1,3-diamine (66), 1,3-diaminopropan-2-one (67), M-methylpropane-1,3-diamine (68), 1,3-bis(dimethylamino)propan-2-ol (69), 1,3-bis(methylamino)propan-2-ol (70), (3-aminopropyl)glycine (71), 2-((3-aminopropyl)amino)ethan-1-ol (72), 2,2′-(propane-1,3-diylbis(azanediyl))bis(ethan-1-ol) (73), 1,4-diazepane (74), 1-amino-3-((2-hydroxyethyl)amino)propan-2-ol (75), 2,2′-((2-hydroxypropane-1,3-diyl)bis(azanediyl))bis(ethan-1-ol) (76), M-(3-aminopropyl)butane-1,4-diamine (77), N¹,N^{r}-(butane-1,4-diyl)bis(propane-1,3-diamine) (78). The bidentate ligands have following chemical structures:

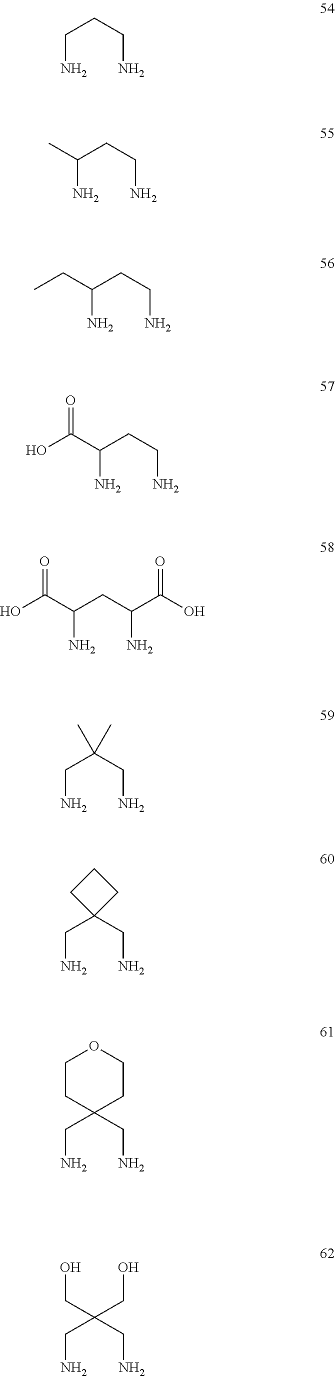

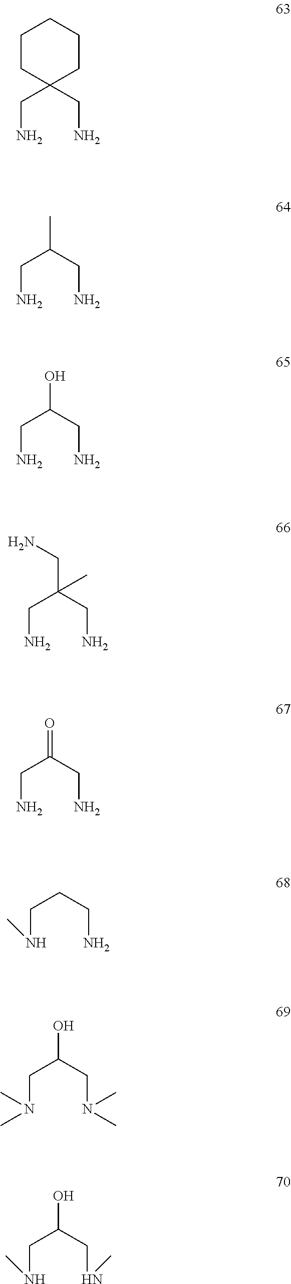

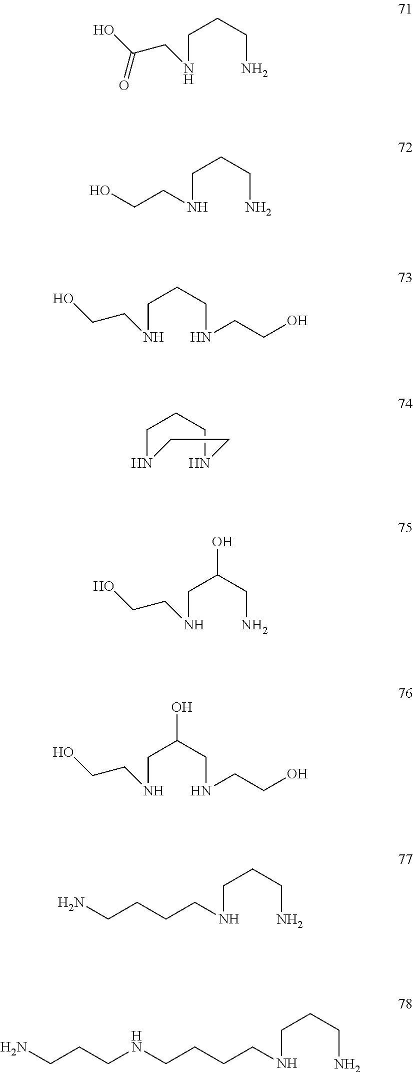

Even further examples of bidentate ligands according to the above mentioned formula are: butane-1,4-diamine (79), 2,5-diaminopentanoic acid (80), 2-methylbutane-1,4-diamine (81), 1,4-diaminobutane-2,3-diol (82), (1,3-dioxolane-4,5-diyl)dimethanamine (83), (2-methyl-1,3-dioxolane-4,5-diyl)dimethanamine (84), (2-ethyl-1,3-dioxolane-4,5-diyl)dimethanamine (85), (2-propyl-1,3-dioxolane-4,5-diyl)dimethanamine (86), (2-isopropyl-1,3-dioxolane-4,5-diyl)dimethanamine (87), (2-phenyl-1,3-dioxolane-4,5-diyl)dimethanamine (88), (2-(2-fluorophenyl)-1,3-dioxolane-4,5-diyl)dimethanamine (89), (2-(3-fluorophenyl)-1,3-dioxolane-4,5-diyl)dimethanamine (90), (2-(4-fluorophenyl)-1,3-dioxolane-4,5-diyl)dimethanamine (91), (2-(thiophen-2-yl)-1,3-dioxolane-4,5-diyl)dimethanamine (92), (2-(furan-2-yl)-1,3-dioxolane-4,5-diyl)dimethanamine (93), cyclobutane-1,2-diyldimethanamine (94), (1s,4s)-cyclohexane-1,4-diamine (95), N¹,N^{1′}-(butane-1,4-diyl)bis(propane-1,3-diamine) (96). The bidentate ligands have following chemical structures:

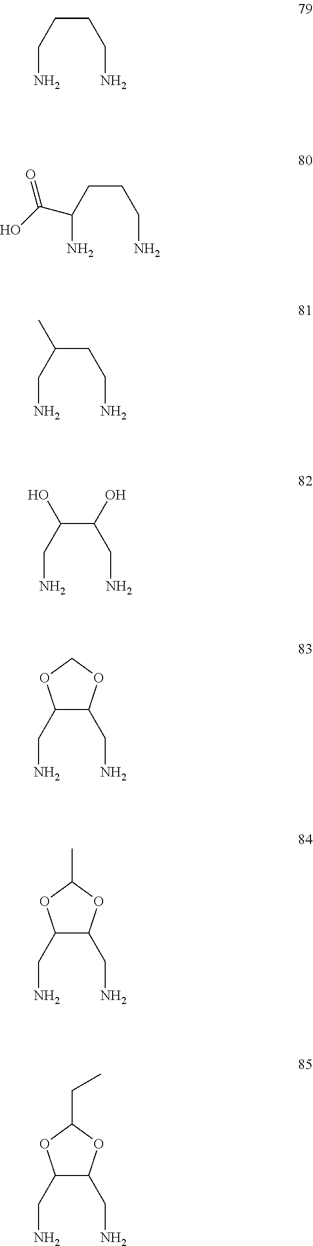

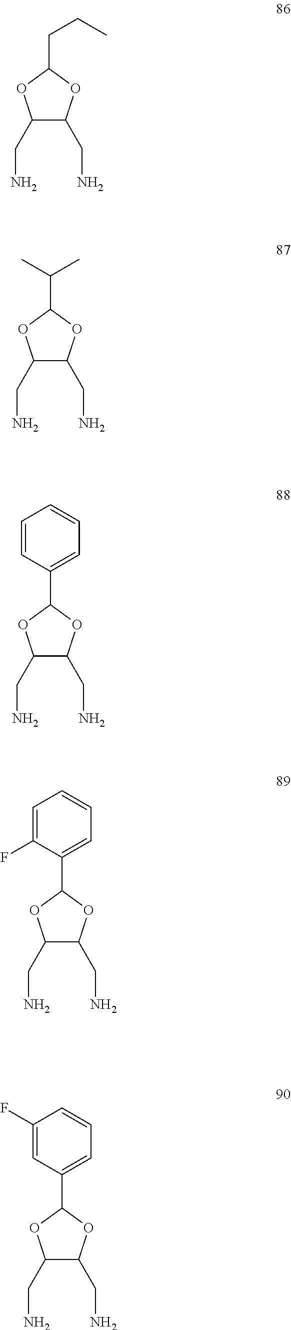 [SMILES: CCCC1OC(CN)C(CN)O1 ; CC(C)C1OC(CN)C(CN)O1 ; NCC1OC(c2ccccc2)OC1CN ; NCC1OC(c2ccccc2F)OC1CN ; NCC1OC(c2cccc(F)c2)OC1CN]

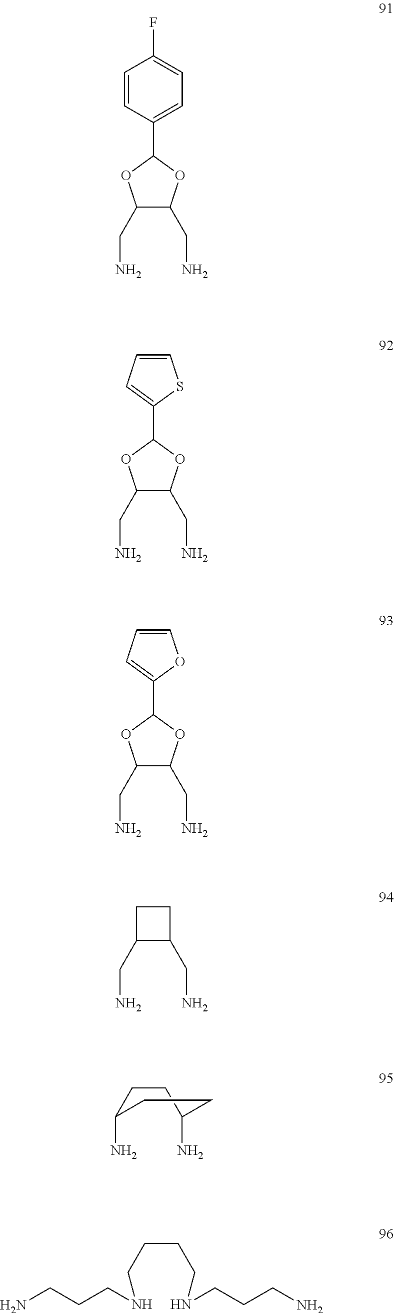 [SMILES: NCC1OC(c2ccc(F)cc2)OC1CN ; NCC1OC(c2cccs2)OC1CN ; NCC1OC(c2ccco2)OC1CN ; NCC1CCC1CN ; NC1CCC(N)CC1 ; NCCCNCCCCNCCCN]

The primary functional moiety, which is part of the secondary functional moiety used in the method of the disclosure is preferably a therapeutic compound, such as a cytotoxic drug, a diagnostic compound, such as a fluorescent dye or a radiotracer ligated to a chelating compound, or a model compound.

It is particularly preferred that the toxic drug is a therapeutic compound that interferes with the cytoskeleton, alkylates the DNA, intercalates into the DNA double helix, inhibits RNA polymerase II or III or inhibits a signal transduction cascade in a cellular system. Most preferably, the primary functional moiety is a cytotoxic compound. Preferred primary toxic moieties are numerous. Several examples of preferred primary toxic moieties hereof are compounds chosen from the group of auristatins, dolastatins, symplostatins, maytansinoids, tubulysins, HTI-286, calicheamycins, duocarmycins, pyrrolobenzodiazepines (PBDs), indolino-benzodiazepines (IGNs), camptothecins, anthracyclines, azonafides, amanitins, cryptophycins, rhizoxins, epothilones, spliceostatins, thailanstatins, colchicines, aplyronines, taxoids, methotrexate, aminopterin, vinca alkaloids. Also preferred toxic moieties are proteinaceous toxins such as a fragment of Pseudomonas exotoxin-A, statins, ricin A, gelonin, saporin, interleukin-2, interleukin-12, viral proteins such as E4, f4, apoptin or NS1, and non-viral proteins such as HAMLET, TRAIL or mda-7.

The primary functional moiety may also be a diagnostic compound. In an alternative embodiment, the functional moiety is a fluorescent dye, such as IRDye800CW, DY-800, ALEXA FLUOR®750, ALEXA FLUOR®790, indocyanine green, FITC, BODIPY dyes such as BODIPY FL and rhodamines such as rhodamine B. Other diagnostic compounds, which may be used in the disclosure as a functional moiety, are radionuclides, PET-imageable agents, SPECT-imageable agents or MRI-imageable agents. It is also possible to couple chelating agents (such as EDTA, DPTA, and deferoxamine (DESFERAL® or DFO)) or macrocyclic agents (such as DOTA or p-SCN-Bn-DOTA) as a functional moiety to the metal ion complex. In a subsequent step, those chelators are loaded with therapeutic or diagnostic radionuclides such as beta emitting agents (such as ⁹⁰Y or ¹⁷⁷Lu), alpha emitters (such as ²¹¹At), PET-itosopes (such as ⁸⁹Zr) or SPECT-istopes (such as⁹⁹mTc), or with non-radioactive metals.

Alternatively, more than one kind of functional moiety can be used. In this way, it is possible to bind different functional moieties, e.g., different useful combinations of therapeutic compounds or different combinations of useful diagnostic compounds or different combinations of both, to one targeting moiety. By doing this, a preferred combination of therapeutic compounds can be delivered to the tissue of interest.

In the case that the second ligand of the secondary functional moiety comprises a chloride as a leaving ligand, an iodide or a bromide releasing agent or their mixture is added to the secondary functional moiety or the conjugation mixture containing the secondary functional moiety and the cell binding moiety, so that chloride is substituted by iodide or bromide. The iodide or bromide releasing agent can be selected from the group comprising NaI, KI, LiI, CsI, RbI, NH₄I, MgI₂, CaI₂, SrI₂, MnI₂, InI₃, AlI₃, GeI₄, guanidinium iodide, tetramethyl ammonium iodide, acetylcholine iodide, 5-(2-hydroxyethyl)-3,4-dimethylthiazolium iodide, trimethylsulfoxonium iodide, NaBr, KBr, LiBr or a mixture thereof, more preferably NaI or KI or a mixture thereof.

If an iodide salt is used the concentration of the salt in the reaction mixture preferably ranges between 0.1 and 100 mM, more preferably between 1 and 30 mM and is most preferably about 10 mM. However, in case a bromide salt is used the concentration thereof in the reaction mixture is preferably about 50 mM.

Furthermore, the pH of the reaction mixture during the binding of the secondary functional moiety to the cell binding moiety to form a cell targeting conjugate preferably ranges between 5.5 and 10.0, more preferably between 7.5 and 8.5, most preferably the pH is about 8.1.

The cell binding moieties used in the methods of the disclosure are preferably antibodies. However, different types may be used, such as single chain antibodies, antibody fragments that specifically bind to a target cell, monoclonal antibodies, engineered monoclonal antibodies, single chain monoclonal antibodies, monoclonal antibodies that specifically bind to a target cell, chimeric antibodies, chimeric antibody fragments that specifically bind to a target cell, and nontraditional protein scaffolds, (e.g., affibodies, anticalins, adnectins, darpins), bicycles or tricycles or folic acid derivatives that specifically bind to the target cells.

Preferably, the cell binding moiety is an antibody selected from the group of immunoglobulins targeting Her2, Her1, CD30, CD20, CD79b, CD19, EGFR, EGFRvIII or PSMA, antibodies directed against intra-cellular targets (such as HLA-MAGE antigen complexes) of aberrant cells (such as tumor cells). More preferably, the cell binding moiety is an antibody selected from the group of immunoglobulins comprising trastuzumab, cetuximab, brentuximab, rituximab, ofatumumab, or obinutuzumab.

In one embodiment, cell targeting conjugates are provided for the specific targeting and killing of aberrant cells, wherein the toxic moiety is linked to cell binding moiety antibody via a transition metal complex. Preferably, the cell targeting conjugates are selected from the group comprising trastuzumab-Pt(ethane-1,2-diamine)-auristatin F, trastuzumab-Pt(ethane-1,2-diamine)-duocarmycin, trastuzumab-Pt(ethane-1,2-diamine)-tubulysin, trastuzumab-Pt(ethane-1,2-diamine)-PBD, trastuzumab-Pt(ethane-1,2-diamine)-maytansinoid, anti-EGFRvIII antibody-Pt(ethane-1,2-diamine)-PNU-159682, anti MAGE-HLA peptide complex antibody-Pt(ethane-1,2-diamine)-alpha-amanitin, anti MAGE-HLA peptide complex antibody-Pt(ethane-1,2-diamine)-PBD, anti MAGE-HLA peptide complex antibody-Pt(ethane-1,2-diamine)-alpha-amanitin, and brentuximab-Pt(ethane-1,2-diamine)-alpha-amanitin.

The concentrations and conditions used in the methods of the disclosure are preferably chosen such that the cell targeting conjugates prepared comprise on average 1-10 functional moieties per cell binding moiety. In case the cell binding moiety is an antibody, this is also referred to as the drug-antibody ratio (DAR).

Hence, the DAR ranges between 1:1 to 10:1, preferably between 1:1 to 5:1.

In the method according to the disclosure the secondary functional moiety is preferably represented by the following formula:

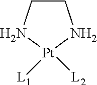

wherein one of the ligands L₁ or L₂ is a leaving ligand chosen from iodide, bromide or chloride, preferably iodide and bromide, and the other ligand is an auristatin derivative such as auristatin E and F or monomethyl auristatin E and F. More preferably auristatin F is used. Furthermore, the secondary functional moiety is preferably bound to trastuzumab according to the methods of the disclosure.

Alternatively, a method according to the disclosure is provided, wherein the secondary functional moiety is preferably represented by the following formula:

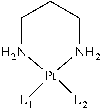

wherein one of the ligands L₁ or L₂ is a leaving ligand chosen from iodide, bromide or chloride, preferably iodide and bromide, and the other ligand is an auristatin derivative such as auristatin E and F or monomethyl auristatin E and F. More preferably auristatin F is used. Furthermore, the secondary functional moiety is preferably bound to trastuzumab according to the methods of the disclosure.

Alternatively, a method according to the disclosure is provided, wherein the secondary functional moiety is preferably represented by the following formula:

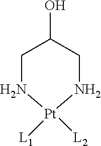

wherein one of the ligands L₁ or L₂ is a leaving ligand chosen from iodide, bromide or chloride, preferably iodide and bromide, and the other ligand is an auristatin derivative such as auristatin E and F or monomethyl auristatin E and F. More preferably auristatin F is used. Furthermore, the secondary functional moiety is preferably bound to trastuzumab according to the methods of the disclosure.

A third aspect of the disclosure relates to cell targeting conjugates obtainable by the method according to the disclosure.

A fourth aspect of the disclosure relates to a pharmaceutical composition comprising the cell targeting conjugates.

A fifth aspect of the disclosure relates to the use of the cell targeting conjugates in the treatment of cancer and other chronic diseases in mammals, in particular, humans. The cell targeting conjugates may be particularly useful in the treatment of colorectal cancer, breast cancer, pancreatic cancer, and non-small cell lung carcinomas. It may be particularly useful to use the cell targeting conjugates according to the disclosure in the treatment of breast cancer, wherein the breast cancer has a low expression level of Her2.

A sixth aspect of the disclosure relates to a composition comprising cell targeting conjugates of the disclosure further comprising a radionuclide such as ^{195m}Pt in the secondary functional moiety. The use of ^{195m}Pt allows the characterization and validation of Lx-based cell targeting conjugates in vivo by using a dual-labeling approach combining ^{195m}Pt counting and ⁸⁹Zr-immuno-PET imaging. The combined use of ⁸⁹Zr and ^{195m}Pt provides the capability of sensitive and direct detection of the Lx linker apart from the antibody and the primary functional moiety, i.a. a drug or a diagnostic agent. The dual labeling strategy can thus demonstrate the in vivo stability of cell targeting conjugates, the in vivo uptake, and the retention of cell targeting conjugates in tumors and normal organs as a function of DAR, and the sequestration of the platinum-based linker (Lx) in the body.

The disclosure will now be elucidated further by means of the following non-limiting examples.

### EXAMPLES

### Example 1: Example of LxCl₂ Complex Used for the Synthesis of Cl-Lx-PFM Complexes (Chlorido Lx-“Semi-Final Products”)

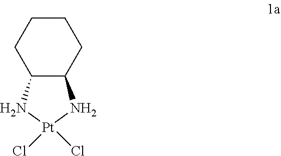

Compound 1a was purchased from Sigma-Aldrich, product code 404322, [52691-24-4].

### Example 2: Example of LxBr₂ Complex Used for the Synthesis of Br-Lx-PFM Complexes (Bromido Lx-“Semi-Final Products”)

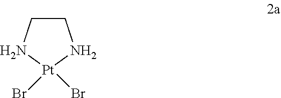

### 2.1. Synthesis and Analytical Characterization of PtBr₂(Ethane-1,2-Diamine) (2a)

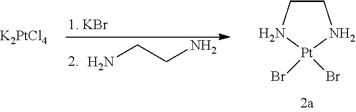

KBr (2.38 g, 20 mmol) was added to a solution of K₂PtCl₄ (415 mg, 1.0 mmol) in water (25 mL). The mixture was stirred at room temperature for 24 h, then the resulting brown mixture was filtered, ethane-1,2-diamine (81 μL, 1.2 mmol) was added to the filtrate, and the mixture was stirred at room temperature for 18 h. The precipitate was collected by filtration, thoroughly washed with water, and dried first under suction on the filter for 1 h. Then, the filter cake (335 mg of a yellow solid) was transferred into a flask and slurry-washed in MeOH (5 mL) for 1 h, collected by filtration, the filter cake was washed with MeOH, and then dried under reduced pressure for 12 h to obtain a yellow solid (298 mg, 72% yield).

Elemental analysis calc for C₂H₈Br₂N₂Pt: C, 5.79; H, 1.94; N, 6.75; found: C, 5.90; H, 1.87; N, 6.63. ¹⁹⁵Pt-NMR (86 MHz, DMF-d₇): δ −2628.

### Example 3: Examples of LxI₂ Complexes Used for the Synthesis of I-Lx-PFM Complexes (Iodido Lx-“Semi-Final Products”)

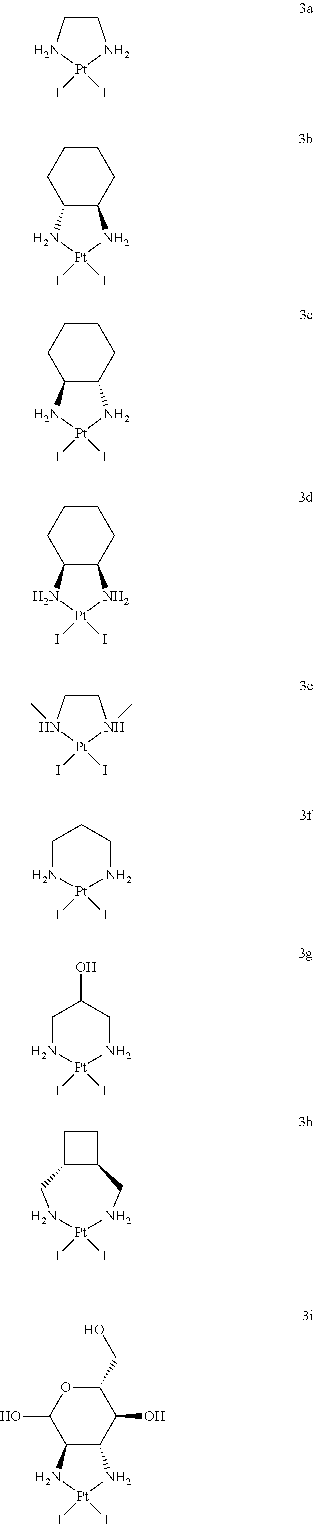

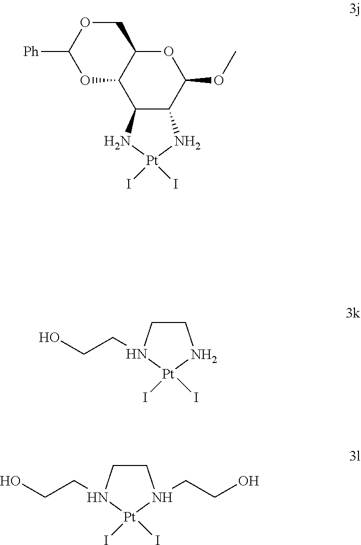

### 3.1. General Synthesis of Complexes PtI₂(Bidentate Ligand) 3a-h and 3j-l (Exemplified for the Complex 3a) and Analytical Data of the Complex Pt(Ethane-1,2-Diamine)I2 (3a)

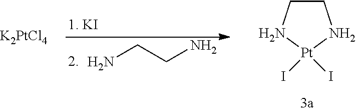

KI (33.2 g, 0.2 mol) was added to a solution of K₂PtCl₄ (4.15 g, 10 mmol) in water (200 mL). The mixture was stirred at room temperature for 22 h, then the resulting dark mixture was filtered, ethane-1,2-diamine (800 μL, 12 mmol) was added to the filtrate, and the mixture was stirred at room temperature for 23 h. A yellow precipitate started to form immediately upon addition of ethane-1,2-diamine. The precipitate was collected by filtration, thoroughly washed with water, and dried first under suction on the filter for 3-4 h and then under reduced pressure for 12 h to obtain a yellow solid (4.85 g, 95% yield).

Elemental analysis calc for C₂H₈I₂N₂Pt: C, 4.72; H, 1.58; N, 5.50; found: C, 4.68; H, 1.44; N, 5.30. ¹⁹⁵Pt-NMR (86 MHz, DMF-d₇): δ −3450. Lit (Inorg. Chem. 1992, 31, p. 5447): −3450.

HPLC (Grace Alltima C18, 25×4.6 mm, 5 μm) indicated that the product was 100% pure (retention time 9.8 min; gradient: 5 to 50% MeCN/0.1% TFA in water/0.1% TFA in 18 min measured at a wavelength of 273 nm).

Following complexes Pt(bidentate ligand)I₂ 3 were obtained in a similar way:

TABLE 1
Obtained complexes Pt(bidentate ligand)I₂ 3

| Complex | Amount of | Amount of | Isolated | Color of |
|---|---|---|---|---|
| 3 | K₂PtCl₄ | bidentate ligand | yield | obtained solid |
| 3b | 830 mg (2.0 mmol) | 280 mg (2.4 mmol) | 1.09 g, 97% | Yellow |
| 3c | 830 mg (2.0 mmol) | 280 mg (2.4 mmol) | 1.08 g, 96% | Yellow |
| 3d | 830 mg (2.0 mmol) | 294 μL (2.4 mmol) | 1.07 g, 95% | Yellow |
| 3e | 830 mg (2.0 mmol) | 261 μL (2.4 mmol) | 1.04 g, 97% | Yellow |
| 3f | 830 mg (2.0 mmol) | 202 μL (2.4 mmol) | 986 mg, 94% | Yellow |
| 3g | 415 mg (1.0 mmol) | 223 mg (2.4 mmol) | 404 mg, 75% | Yellow |
| 3h | 830 mg (2.0 mmol) | 248 μL (2.0 mmol) | 1.03 g, 91% | Beige-yellow |
| 3j | 74 mg (0.18 mmol) | 50 mg (0.18 mmol)¹ | 123 mg, 95% | Orange |
| 3k | 830 mg (2.0 mmol) | 252 mg (2.4 mmol)² | 1.02 g, 92% | Yellow |
| 3l | 830 mg (2.0 mmol) | 367 mg (2.4 mmol) | 960 mg, 80% | Yellow-orange |

¹dissolved in MeOH before addition
²dissolved in water before addition

3.1.1. Analytical data of the complex Pt((1R,2R)-cyclohexane-1,2-diamine)I₂ (3b)

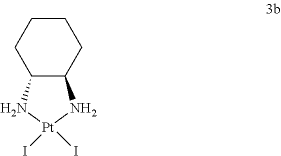

Elemental analysis calc for C₆H₁₄I₂N₂Pt: C, 12.80; H, 2.51; N, 4.98; found: C, 12.77; H, 2.42; N, 4.79. ¹⁹⁵Pt-NMR (86 MHz, DMF-d₇): δ −3421.

### 3.1.2. Analytical Data of the Complex Pt((1S,2S)-Cyclohexane-1,2-Diamine)I₂ (3c)

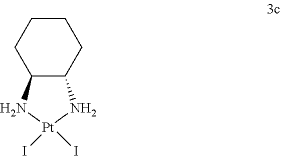

Elemental analysis calc for C₆H₁₄I₂N₂Pt: C, 12.80; H, 2.51; N, 4.98; found: C, 12.71; H, 2.35; N, 4.85.

### 3.1.3. Analytical Data of the Complex Pt((1R,2S)-cyclohexane-1,2-diamine)I₂ (3d)

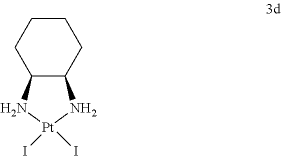

Elemental analysis calc for C₆H₁₄I₂N₂Pt: C, 12.80; H, 2.51; N, 4.98; found: C, 12.90; H, 2.36; N, 4.78. ¹⁹⁵Pt-NMR (86 MHz, DMF-d₇): 8-3399.

### 3.1.4. Analytical Data of the Complex Pt(N¹,N²-dimethylethane-1,2-diamine)I₂ (3e)

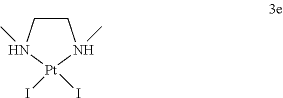

Elemental analysis calc for C₄H₁₂I₂N₂Pt: C, 8.95; H, 2.25; N, 5.22; found: C, 8.83; H, 2.08; N, 5.06. ¹⁹⁵Pt-NMR (86 MHz, DMF-d₇): δ −3431.

### 3.1.5. Analytical Data of the Complex PtI₂(propane-1,3-diamine) (3f)

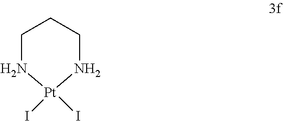

After isolation and initial drying step, the material was additionally slurry-washed in MeOH, filtered, washed with MeOH, and dried.

Elemental analysis calc for C₃H₁₀I₂N₂Pt: C, 6.89; H, 1.93; N, 5.36; found: C, 6.91; H, 1.85; N, 5.13. ¹⁹⁵Pt-NMR (86 MHz, DMF-d₇): 8-3330.

HPLC (Grace Alltima C18, 25×4.6 mm, 5 μm) indicated that the product was 100% pure (retention time 13.6 min; gradient: 5 to 50% MeCN/0.1% TFA in water/0.1% TFA in 18 min measured at a wavelength of 223 nm).

### 3.1.6. Analytical Data of the Complex Pt(1,3-diaminopropan-2-ol)I₂ (3g)

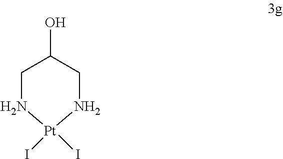

After isolation and initial drying step, the material was additionally slurry-washed in MeOH, filtered, washed with MeOH, and dried.

Elemental analysis calc for C₃H₁₀I₂N₂OPt: C, 6.68; H, 1.87; N, 5.20; found: C, 6.76; H, 1.78; N, 4.91. ¹⁹⁵Pt-NMR (86 MHz, DMF-d₇): 8-3354.

HPLC (Grace Alltima C18, 25×4.6 mm, 5 μm) indicated that the product was 100% pure (retention time 12.1 min; gradient: 5 to 50% MeCN/0.1% TFA in water/0.1% TFA in 18 min measured at a wavelength of 273 nm).

### 3.1.7. Analytical Data of the Complex Pt((1R,2R)-cyclobutane-1,2-diyl)dimethanamine)I₂ (3h)

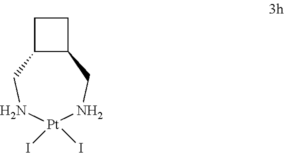

After isolation and initial drying step, the material was additionally slurry-washed in MeOH, filtered, washed with MeOH, and dried.

Elemental analysis calc for C₆H₁₄I₂N₂Pt: C, 12.80; H, 2.51; N, 4.98; found: C, 12.99; H, 2.43; N, 4.68. ¹⁹⁵Pt-NMR (86 MHz, DMF-d₇): 8-3325.

### 3.1.8. Analytical data of the complex Pt((4aR,6R,7R,8R,8aS)-6-methoxy-2-phenylhexahydropyrano[3,2-d][1,3]dioxine-7,8-diamine)I₂ (3j)

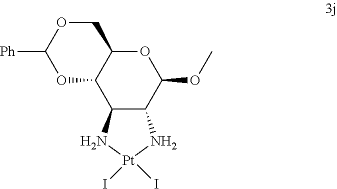

Elemental analysis calc for C₁₄H₂₀I₂N₂O₄Pt: C, 23.06; H, 2.76; N, 3.84; found: C, 23.09; H, 2.65; N, 3.73. ¹⁹⁵Pt-NMR (86 MHz, DMF-d₇): δ −3434.

### 3.1.9. Analytical Data of the Complex Pt(2-((2-aminoethyl)amino)ethan-1-ol)I₂ (3k)

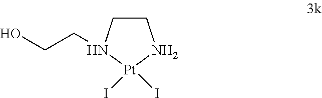

Elemental analysis calc for C₄H₁₂I₂N₂OPt: C, 8.69; H, 2.19; N, 5.07; found: C, 8.69; H, 2.06; N, 4.88. ¹⁹⁵Pt-NMR (86 MHz, DMF-d₇): δ −3438.

HPLC (Grace Alltima C18, 25×4.6 mm, 5 μm) indicated that the product was 100% pure (retention time 11.2 min; gradient: 5 to 50% MeCN/0.1% TFA in water/0.1% TFA in 18 min measured at a wavelength of 273 nm).

### 3.1.10. Analytical Data of the Complex Pt(2,2′-(ethane-1,2-diylbis(azanediyl))bis(ethan-1-ol))I₂ (3l)

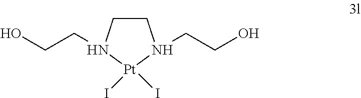

Elemental analysis calc for C₆H₁₆I₂N₂O₂Pt: C, 12.07; H, 2.70; N, 4.69; found: C, 12.03; H, 2.58; N, 4.44. ¹⁹⁵Pt-NMR (86 MHz, DMF-d₇): δ −3443.

### 3.2. Synthesis of the Complex Pt((3R,4R,5S,6R)-3,4-diamino-6-(hydroxymethyl)tetrahydro-2H-pyran-2,5-diol)I₂ (3i)

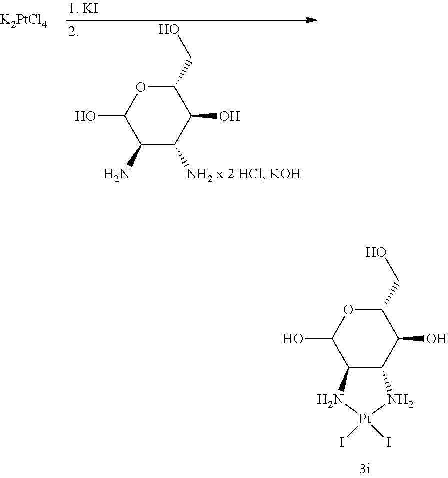

Prepared according to Berger et al., ChemMedChem 2007, 2, 505-514.

KI (531 mg, 3.2 mmol) was added to a solution of K₂PtCl₄ (266 mg, 0.64 mmol) in water (1.3 mL). The mixture was stirred at room temperature for 30 min, then the resulting dark mixture was filtered, and a solution of (3R,4R,5S,6R)-3,4-diamino-6-(hydroxymethyl)tetrahydro-2H-pyran-2,5-diol dihydrochloride (250 mg, 1.0 mmol) and KOH (98 mg, 1.5 mmol) in water (400 μL), filtered through a pad of Celite, was added to the filtrate. The mixture was stirred at room temperature for 22 h. A precipitate started to form immediately upon addition of the solution of (3R,4R,5S,6R)-3,4-diamino-6-(hydroxymethyl)tetrahydro-2H-pyran-2,5-diol. The precipitate was collected by filtration, washed with cold water (1.5 mL), followed by cold acetone (1 mL), and dried first under suction on the filter for 1 h and then under reduced pressure for 12 h to obtain a dark brown solid (162 mg, 43% yield).

¹⁹⁵Pt-NMR (86 MHz, DMF-d₇): δ−3423, −3430 (mixture of epimers).

### Example 4: Examples of Chlorido Lx-“Semi-Final Products” Cl-Lx-PFM (Chlorido SFMs)

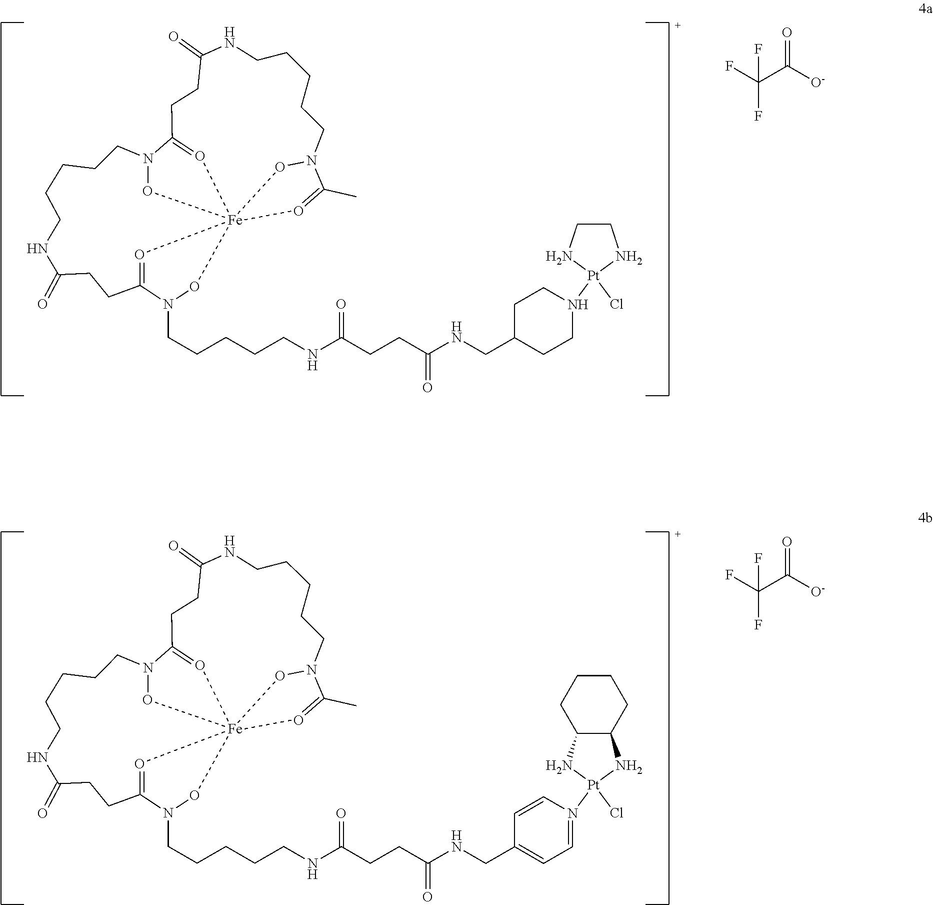

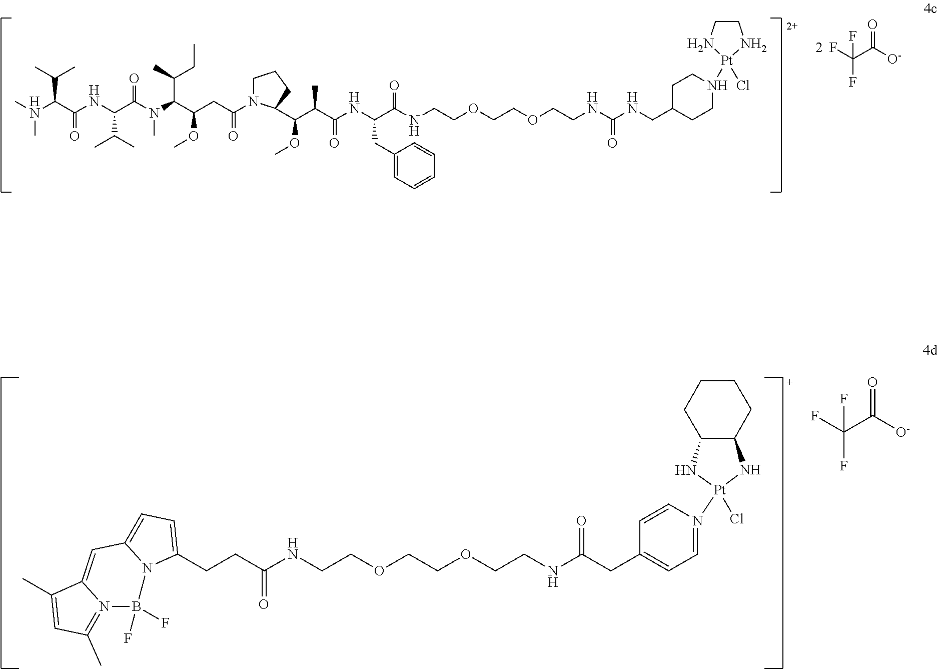

### 4.1. Synthesis and Analytical Characterization of [PtCl((Fe)DFO-pip)(ethane-1,2-diamine)]⁺ TFA⁻ (4a) is Described in Sijbrandi et al., Cancer Res. 2017, 72, 257-267

### 4.2. Synthesis and Analytical Characterization of [PtCl((Fe)DFO-suc-py)((1R,2R)-(−)-1,2-diaminocyclohexane)]⁺ TFA⁻ (4b)

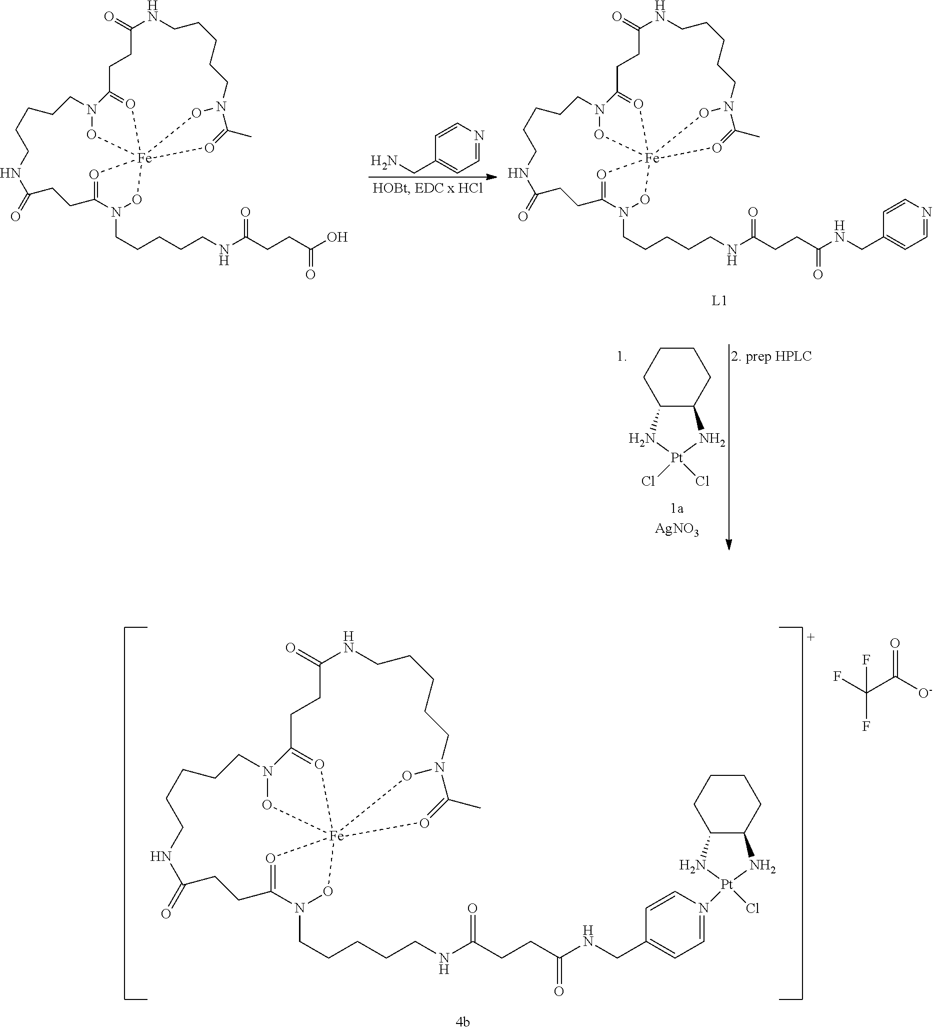

### 4.2.1. Synthesis of the Ligand (Fe)DFO-suc-py (L1)

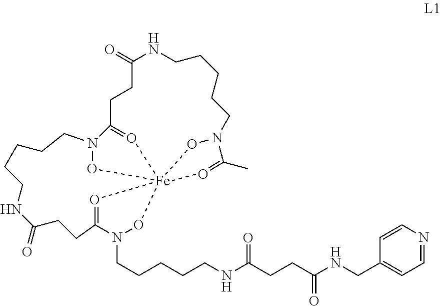

Prepared according to Verel et al., J. Nucl. Med. 2003, 44, 1271-1281.

N-Succinyl Desferal-Fe(III) ((Fe)DFO-suc; 89 mg, 124 μmol) was dissolved in DMF (1.2 mL) and HOBt (25.2 mg, 186 μmol), EDC×HCl (35.7 mg, 186 μmol), DIPEA (43 μL, 248 μmol) and pyridin-4-ylmethanamine (14 μL, 137 μmol) were sequentially added. The mixture was stirred for 20 h, concentrated, and the residue was dissolved in water and purified by Sep-Pak C18 Plus columns. The product was eluted from the columns and lyophilized resulting in a dark red solid (124 mg, 83% yield).

HRMS (ESI⁺) C₃₅H₅₆FeN₈O₁₀ [M+H]⁺ calc 804.3463, found 804.3516

### 4.2.2. Synthesis of the Complex [PtCl((Fe)DFO-suc-py)((1R,2R)-(−)-1,2-diaminocyclohexane)]⁺ TFA⁻ (4b)

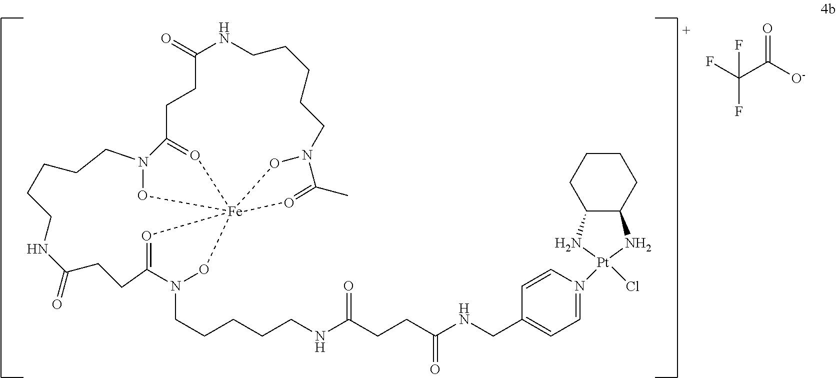

AgNO₃ (41 mg, 0.241 mmol) was added to a suspension of PtCl₂((1R,2R)-(−)-1,2-diaminocyclohexane) (1a) (87 mg, 0.229 mmol) in DMF (1 mL). After stirring for 24 h, the grey precipitate was filtered through Celite, which was then rinsed with DMF (2×0.5 mL). Then, 357 μL of this solution (1.1 eq. of activated Pt-complex) were added to (Fe)DFO-suc-py (L1) (30 mg, 0.037 mmol). The mixture was stirred for 24 h under argon after which HPLC indicated full conversion. The solvent was evaporated under reduced pressure, after which the residue was dissolved in a mixture of water and methanol. Purification was performed by preparative reverse-phase HPLC (Grace Alltima C18 5 μm column, 22×250 mm; gradient: 15 to 25% MeCN/0.1% TFA in water/0.1% TFA in 36 min). Product fractions were collected on ice and immediately frozen and lyophilized resulting in a dark red solid (10 mg, 21% yield).

HRMS (ESI⁺) C₄₁H₆₉³⁵ClFeN₁₀O₁₀¹⁹⁵Pt[M]⁺ calc 1147.3885, found 1147.3672; C₄₁H₆₉³⁵ClFeN₁₀N_{aO10}¹⁹⁵Pt[M+Na]²⁺ calc 585.1891, found 585.1771

HPLC (Grace Alltima C18 5 μm column, 25×4.6 mm) indicated that the product was 97.2% pure (retention time 14.2 min; gradient: 5 to 50% MeCN/0.1% TFA in water/0.1% TFA in 18 min measured at a wavelength of 430 nm).

### 4.3. Synthesis and Analytical Characterization of Pt(auristatin F-(4-(12-amino-3-oxo-7,10-dioxa-2,4-diazadodecyl)piperidine))C₁(ethane-1,2-diamine) (4c) is Described in Sijbrandi et al., Cancer Res. 2017, 72, 257-267

### 4.4. Synthesis and Analytical Characterization of [BODIPY FL-PEG₂-py-PtCl((1R,2R)-(−)-1,2-diaminocyclohexane)]⁺ TFA⁻ (4d)

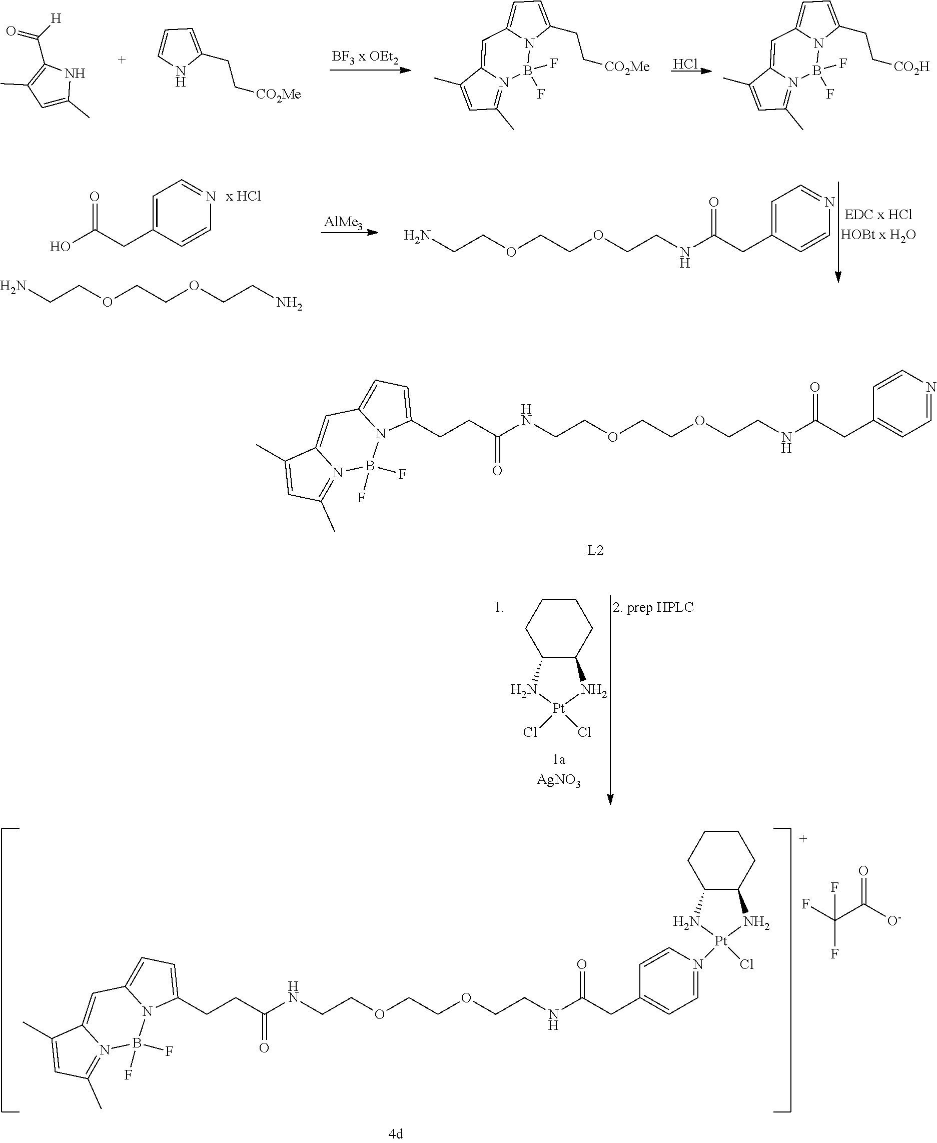

### 4.4.1. Synthesis of BODIPY FL Methyl Ester

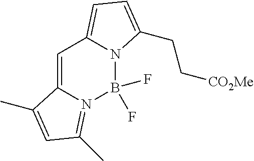

Prepared according to GieBler et al., Eur. J. Org. Chem 2010, 3611-3620.

Methyl 3-(1H-pyrrol-2-yl)propanoate (780 mg, 4.84 mmol, 1.0 eq.) and 3,5-dimethyl-1H-pyrrole-2-carbaldehyde (690 mg, 5.32 mmol, 1.1 eq.) were dissolved in DCM (50 mL) and cooled to 0° C. To this mixture, a solution of POCl₃ (500 μL, 5.36 mmol, 1.1 eq.) in DCM (5 mL) was added dropwise. The reaction mixture was stirred for 30 min at 0° C. and for 6 h at room temperature. The resulting black solution was again cooled to 0° C. and treated with BF₃×OEt₂ (2.4 mL, 19.5 mmol, 4.0 eq.) and DIPEA (3.5 mL, 20.1 mmol, 4.2 eq.) and stirred for 12 h with gradual warming to room temperature. Then, the mixture was cooled to 0° C. and water (100 mL) was added. The mixture was filtered through Celite, which was rinsed with DCM (4×25 mL), the filtrate phases were separated and the aqueous layer was extracted with DCM (3×50 mL). The combined organic layers were dried with sodium sulfate and the solvents were removed under reduced pressure. The residue was absorbed on Celite and purified by column chromatography (eluent: 10-0% petroleum ether/DCM) to afford a red solid (1.00 g, 68% yield).

¹H NMR (400 MHz, CDCl₃): δ 7.08 (s, 1H), 6.88 (d, J=3.4 Hz, 1H), 6.26 (d, J=3.6 Hz, 1H), 6.11 (s, 1H), 3.69 (s, 3H), 3.29 (t, J=7.6 Hz, 2H), 2.77 (t, J=7.6 Hz, 2H), 2.56 (s, 3H), 2.25 (s, 3H).

### 4.4.2. Synthesis of BODIPY FL

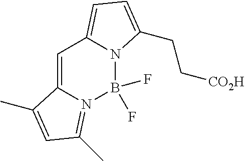

Prepared according to GieBler et al., Eur. J Org. Chem 2010, 3611-3620.

The BODIPY methyl ester (494 mg, 1.61 mmol) was dissolved in THF (75 mL) and 4.5 M HCl (75 mL). This mixture was stirred for 47 h at room temperature. Subsequently, DCM (300 mL) was added and the phases were separated. The aqueous layer was extracted with DCM (100 mL), the combined organic layers were dried with sodium sulfate and the solvents were removed under reduced pressure. The residue was purified by column chromatography (eluent: 0-0.5% MeOH/DCM+0.1% AcOH), followed by precipitation with n-pentane to afford a red solid (276 mg, 59% yield).

¹H NMR (400 MHz, CDCl₃): δ 10.1 (br s, 1H), 7.09 (s, 1H), 6.88 (d, J=3.4 Hz, 1H), 6.29 (d, J=3.6 Hz, 1H), 6.12 (s, 1H), 3.30 (t, J=7.6 Hz, 2H), 2.83 (t, J=7.6 Hz, 2H), 2.57 (s, 3H), 2.25 (s, 3H).

### 4.4.3. Synthesis of N-(2-(2-(2-aminoethoxy)ethoxy)ethyl)-2-(pyridin-4-yl)acetamide (PEG₂-py spacer)

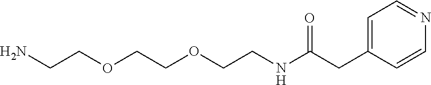 [SMILES: NCCOCCOCCNC(=O)Cc1ccncc1]

2-(Pyridin-4-yl)acetic acid hydrochloride (183 mg, 1.0 mmol, 1.0 eq.) and 2,2′-(ethane-1,2-diylbis(oxy))diethanamine (747 L, 5.0 mmol, 5.0 eq.) were dissolved in dry and degassed toluene (5 mL). Subsequently, a 2 M solution of AlMe₃ in toluene (0.5 mL, 1.0 mmol, 1.0 eq.) was added and the resulting reaction mixture was stirred for 1 h at 90° C. The reaction mixture was then allowed to cool to room temperature over the course of 1 h and was cooled further to 0° C., followed by the addition of isopropanol (1 mL) and a 7 M solution of NH₃ in MeOH (0.14 mL), and warmed to room temperature. The yellow mixture was filtered and the solvents were removed under reduced pressure to give a green oil. This oil was dissolved in DCM and the formed precipitate was again removed by filtration. The solvent was removed under reduced pressure, after which the residue was purified by column chromatography (eluent: DCM/MeOH/NH_{3 aq}. 100:9:1 to 100:9:1.5) to afford a pale yellow oil (129 mg, 48% yield).

HRMS (ESI⁺) C₁₃H₂₂N₃O₃ [M+H]⁺ calc 268.1656, found 268.1645.

¹H NMR (400 MHz, CDCl₃): δ 8.55-8.52 (m, 2H), 7.25-7.22 (m, 2H), 6.67 (s, 1H), 3.59-3.56 (m, 4H), 3.55-3.47 (m, 6H), 3.47-3.42 (m, 2H), 2.88-2.83 (m, 2H), 1.76 (s, 2H).

HPLC (Grace Alltima C18 5 μm column, 25×4.6 mm) indicated that the product was 100% pure (retention time 15.2 min; gradient: 20 to 100% MeCN/0.1% TFA in water/0.1% TFA in 20 min measured at a wavelength of 210 nm).

### 4.4.4. Synthesis of BODIPY FL-PEG₂-py Ligand (L2)

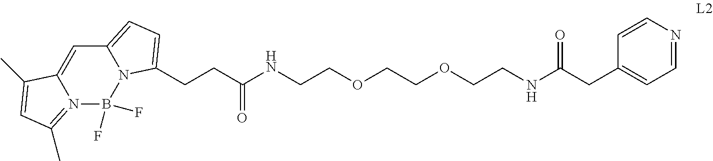

BODIPY FL (33 mg, 112 μmol, 1.0 eq.), EDC×HCl (24 mg, 123 μmol, 1.1 eq.), and HOBt hydrate (19 mg, 123 μmol, 1.1 eq.) where dissolved in DCM (1 mL) and stirred for 5 min. To this mixture PEG₂-py spacer (30 mg, 112 μmol, 1.0 eq.) was added, followed by DIPEA (41.0 μL, 236 μmol, 2.1 eq.), and the mixture was stirred for 18 h at room temperature. Subsequently, the mixture was diluted with DCM (25 mL) and washed with 0.14 M NaOH (32 mL). The two phases were separated, the aqueous layer was extracted with DCM (5×5 mL), and the combined organic layers were dried with sodium sulfate. The solvent was removed under reduced pressure and the residue was purified by column chromatography (eluent: 1-5.5% MeOH in DCM) to obtain a red oil (30 mg, 49% yield).

HRMS (ESI⁺) C₂₇H₃₅BF₂N₅O₄ [M+H]⁺ calc 542.2745, found 542.2755.

¹H NMR (250 MHz, CDCl₃): δ 8.5 (br s, 2H), 7.23-7.18 (m, 2H), 7.06 (s, 1H), 6.89-6.85 (m, 1H), 6.49-6.40 (m, 1H), 6.30-6.26 (m, 2H), 6.11 (s, 1H), 3.54-3.36 (m, 14H), 3.27 (t, J=7.6 Hz, 2H), 2.66-2.58 (m, 2H), 2.53 (s, 3H), 2.24 (s, 3H).

HPLC (Grace Alltima C18 5 μm column, 25×4.6 mm) indicated that the product was 100% pure (retention time 10.2 min; gradient: 20 to 100% MeCN/0.1% TFA in water/0.1% TFA 20 min measured at a wavelength of 488 nm).

### 4.4.5. Synthesis of [BODIPY FL-PEG₂-py-PtCl((1R,2R)-(−)-1,2-diaminocyclohexane)]⁺ TFA⁻ (4d)

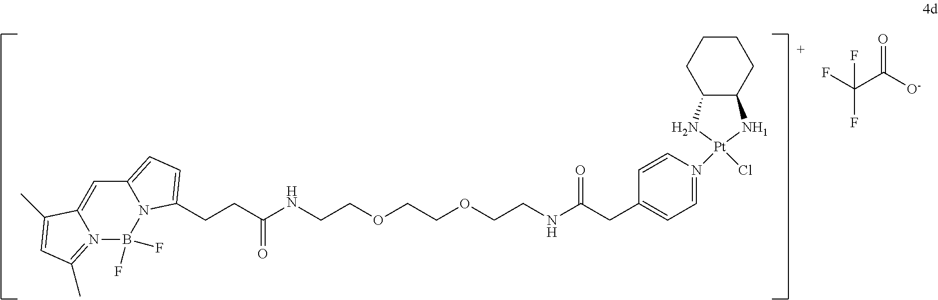

PtCl₂((1R,2R)-(−)-1,2-diaminocyclohexane) (1a) (50 mg, 131 μmol) and AgNO₃ (26 mg, 153 μmol) were dissolved in dry DMF (10 mL) under argon atmosphere and stirred for 22 h at room temperature under light exclusion (the reaction flask has been darkened). Subsequently, the mixture was filtered through a 0.2 μm syringe filter, to give a 13.2 mM stock solution of activated Pt-complex. Then, to the solution of BODIPY FL-PEG₂-py (L2) (14 mg, 26 μmol, 1.0 eq.) in DMF (200 μL), the 13.2 mM stock solution of activated Pt-complex (5.20 mL, 68.4 μmol, 2.6 eq.) was added, followed by triethylamine (7.21 μL, 52 μmol, 2.0 eq.), and the course of the reaction was followed by HPLC. The reaction mixture was stirred for 5 h at room temperature under light exclusion (the reaction flask has been darkened). At this moment, the reaction mixture contained 64.7% product and no starting material.

The mixture was concentrated under reduced pressure, diluted with water/MeOH (2.5:1, 2.5 mL), and filtered through a 0.2 μm syringe filter. Purification was performed by preparative reverse-phase HPLC (Grace Alltima C18 5 μm column, 22×250 mm; gradient: 35 to 85% MeOH/0.1% TFA in water/0.1% TFA in 36 min). Product fractions were lyophilized resulting in a bright orange solid (13 mg, 50% yield).

HRMS (ESI⁺) C₃₃H₄₈B³⁵ClF₂N₇O₄¹⁹⁵Pt[M]⁺ calc 885.3160, found 885.3162

HPLC (Grace Alltima C18 5 μm column, 25×4.6 mm) indicated that the product was 93.6% pure (retention time 12.2 min; gradient: 20 to 100% MeCN/0.1% TFA in water/0.1% TFA in 20 min measured at a wavelength of 488 nm).

### Example 5: Examples of Bromido Lx-“Semi-Final Products” Br-Lx-PFM (Bromido SFMs)

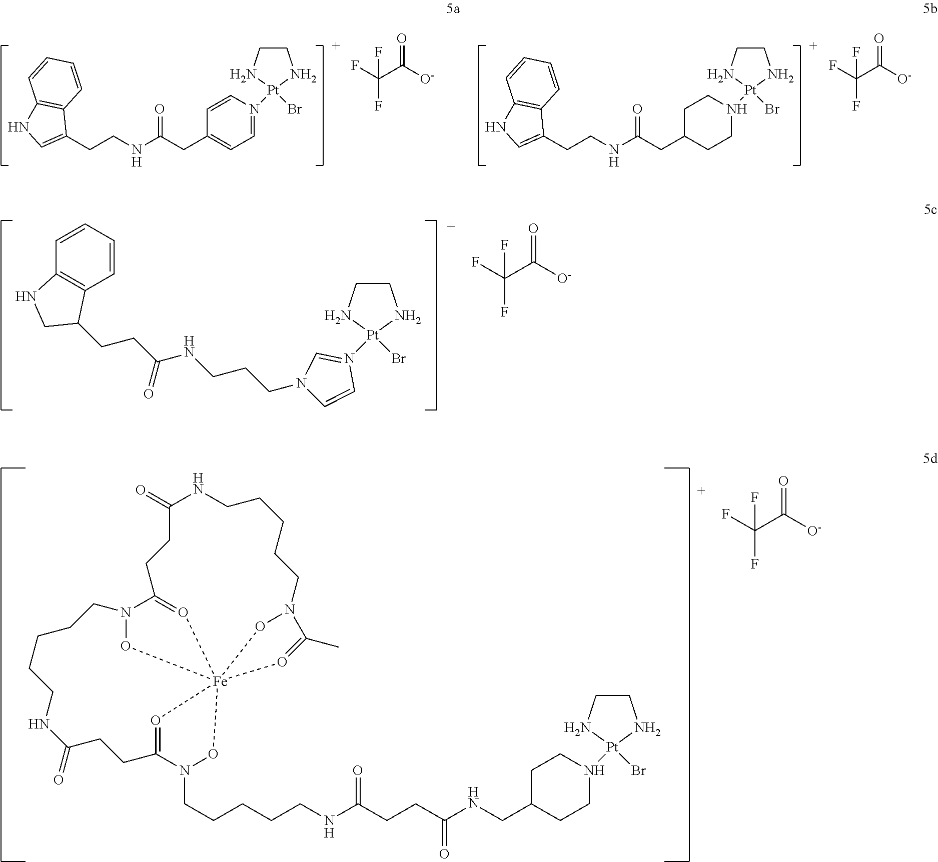

### 5.1. Synthesis and Analytical Characterization of [ind-py-PtBr(ethane-1,2-diamine)]⁺ TFA⁻ (5a)

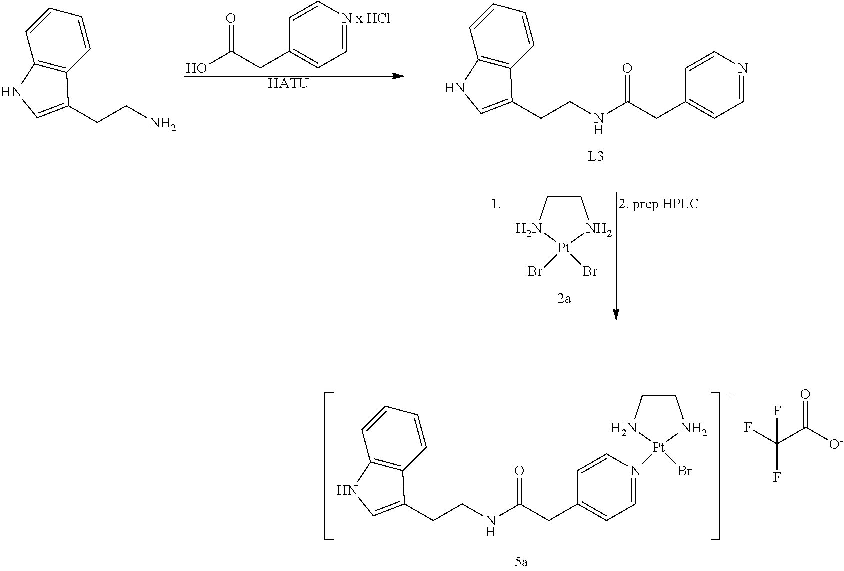

### 5.1.1. Synthesis of the Ligand N-(2-(1H-indol-3-yl)ethyl)-2-(pyridin-4-yl)acetamide (ind-py, L3)

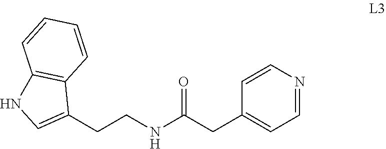 [SMILES: O=C(Cc1ccncc1)NCCc1c[nH]c2ccccc12]

2-(Pyridin-4-yl)acetic acid hydrochloride (365 mg, 2.0 mmol) was suspended in dry DMF (5 mL) and tryptamine (392 mg, 2.4 mmol) was added, followed by the addition of HATU (1.16 g, 4.0 mmol) and DIPEA (1.4 mL, 8 mmol). After stirring at room temperature for 24 h, the mixture was diluted with water, extracted with DCM, and after removal of solvents under reduced pressure the residue was absorbed on Celite and purified chromatographically on silica (eluent: DCM/MeOH/NH_{3 aq.}=100:1:1 to 100:2:1 to 100:3:1). After drying, an orange glass (388 mg, 70% yield) was obtained.

HRMS (ESI⁺) C₁₇H₁₈N₃O [M+H]⁺ calc 280.1460, found 280.1444

HPLC (Grace Alltima C18 5 μm column, 25×4.6 mm) indicated that the product was 98.5% pure (retention time 14.9 min; gradient: 5 to 50% MeCN/0.1% TFA in water/0.1% TFA in 18 min measured at a wavelength of 273 nm).

### 5.1.2. Synthesis of the Complex [ind-py-PtBr(ethane-1,2-diamine)]⁺ TFA⁻ (5a)

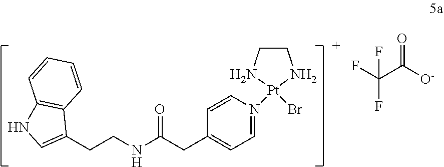

N-(2-(1H-Indol-3-yl)ethyl)-2-(pyridin-4-yl)acetamide (L3) (ind-py; 14.0 mg, 50 μmol, 1.0 eq.) and PtBr₂(ethane-1,2-diamine) (2a) (31.1 mg, 75 μmol, 1.5 eq.) were dissolved in dry DMF (500 μL) under argon atmosphere. Triethylamine (10.5 μL, 75 μmol, 1.5 eq.) was added and the course of the reaction was followed by HPLC. The reaction mixture was stirred at 60° C. for 42 h, then the temperature was increased to 70° C. and the reaction mixture was stirred for an additional 20 h. At this moment, the reaction mixture contained 94.4% product and 1.2% starting material.

The reaction mixture was diluted with water/MeOH (4:1, 2.5 mL) and filtered through a 0.2 μm syringe filter. Purification was performed by preparative reverse-phase HPLC (Grace Alltima C18 5 μm column, 22×250 mm; gradient: 35 to 70% MeOH/0.1% TFA in water/0.1% TFA in 36 min). Product fractions were lyophilized resulting in a colorless solid (12.9 mg, 35.5% yield).

HRMS (ESI⁺) C₁₉H₂₅⁷⁹BrN₅O¹⁹⁵Pt[M]⁺ calc 613.0886, found 613.0877

HPLC (Grace Alltima C18 5 μm column, 25×4.6 mm) indicated that the product was 98.8% pure (retention time 17.8 min; gradient: 5 to 50% MeCN/0.1% TFA in water/0.1% TFA in 18 min measured at a wavelength of 223 nm).

### 5.2. Synthesis and Analytical Characterization of ind-pip-PtBr(ethane-1,2-diamine) (5b)

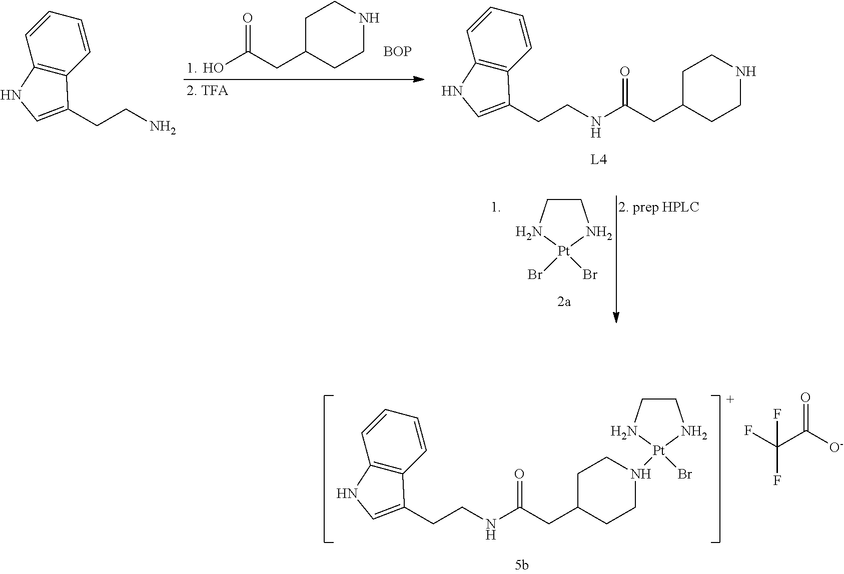

### 5.2.1. Synthesis of the Ligand N-(2-(1H-indol-3-yl)ethyl)-2-(piperidin-4-yl)acetamide (ind-pip, L₄)

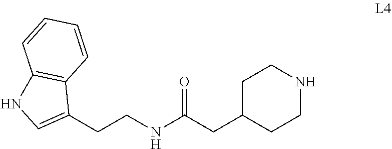 [SMILES: O=C(CC1CCNCC1)NCCc1c[nH]c2ccccc12]

Tryptamine (491 mg, 3.0 mmol, 1.0 eq.) was dissolved in DMF (5 mL). BOP (1.37 g, 3.0 mmol, 1.0 eq.), dissolved in DMF (5 mL), and DIPEA (523 μL, 3.0 mmol, 1.0 eq.) were added, followed by the addition of a solution of 2-(1-(tert-butoxycarbonyl)piperidin-4-yl)acetic acid (745 mg, 3.0 mmol, 1.0 eq.) in DMF (5 mL). After stirring at room temperature for 24 h, the mixture was diluted with water (15 mL), extracted with DCM (3×15 mL), and after removal of solvents under reduced pressure the residue was absorbed on Celite and purified chromatographically on silica using ethyl acetate/cyclohexane 1:1 as an eluent. After drying under reduced pressure, a brown oil (˜2.1 g) was obtained.

TFA (5 mL) was added to the material and the mixture was stirred at room temperature for 30 min, after which it was added slowly into an ice/water cooled 1 N NaOH (50 mL) solution. DCM was added and the mixture was stirred at 0° C. After addition of a small amount of MeOH the phases were separated and the aqueous layer was extracted with dichloromethane (9×25 mL). After evaporation, the residue (˜1.2 g of a brown oil) was absorbed on Celite and purified chromatographically on silica (eluent: isopropanol/NH_{3 aq.}=100:1 to 100:2 to 100:3 to 100:4). The obtained material was then recrystallized from MeOH/dichloromethane/n-pentane and after drying a colorless solid (204 mg, 24% yield) was obtained.

HRMS (ESI⁺) C₁₇H₂₄N₃₀ [M+H]⁺ calc 286.1914, found 286.1920

¹H NMR (400 MHz, DMSO-d₆): δ 10.80 (s, 1H, NH), 7.93-7.87 (m, 1H, NH), 7.55-7.50 (m, 1H), 7.35-7.31 (m, 1H), 7.12 (d, J=1.7 Hz, 1H), 7.09-7.03 (m, 1H), 7.00-6.94 (m, 1H), 3.36-3.28 (m, 2H), 2.94-2.84 (m, 2H), 2.84-2.77 (m, 2H), 2.48-2.38 (m, 2H), 2.00-1.93 (m, 2H), 1.85-1.66 (m, 1H), 1.58-1.46 (m, 2H), 1.15-0.94 (m, 2H).

HPLC (Grace Alltima C18 5 μm column, 25×4.6 mm) indicated that the product was 100% pure (retention time 15.1 min; gradient: 5 to 50% MeCN/0.1% TFA in water/0.1% TFA in 18 min measured at a wavelength of 273 nm).

### 5.2.2. Synthesis of the Complex [ind-pip-PtBr(ethane-1,2-diamine)]⁺ TFA⁻ (5b)

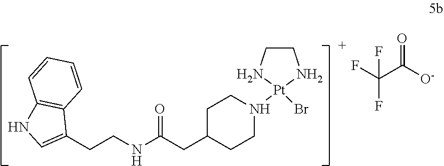

N-(2-(1H-Indol-3-yl)ethyl)-2-(piperidin-4-yl)acetamide (L4) (ind-pip; 14.3 mg, 50 μmol, 1.0 eq.) and PtBr₂(ethane-1,2-diamine) (2a) (20.8 mg, 50 μmol, 1.0 eq.) were dissolved in dry DMF (333 μL) under argon atmosphere. Triethylamine (6.98 μL, 50 μmol, 1.0 eq.) was added and the course of the reaction was followed by HPLC. The reaction mixture was stirred at 60° C. for 42 h. At this moment, the reaction mixture contained 88.6% product and maximally 2.6% starting material.

The reaction mixture was diluted with water/MeOH (4:1, 2.5 mL) and filtered through a 0.2 μm syringe filter. Purification was performed by preparative reverse-phase HPLC (Grace Alltima C18 5 μm column, 22×250 mm; gradient: 35 to 70% MeOH/0.1% TFA in water/0.1% TFA in 36 min). Product fractions were lyophilized resulting in a colorless solid (9.0 mg, 24.5% yield).

HRMS (ESI⁺) C₁₉H₃₁⁷⁹BrN₅O¹⁹⁵Pt[M]⁺ calc 619.1355, found 619.1353

HPLC (Grace Alltima C18 5 μm column, 25×4.6 mm) indicated that the product was 95.6% pure (retention time 17.4 min; gradient: 5 to 50% MeCN/0.1% TFA in water/0.1% TFA in 18 min measured at a wavelength of 223 nm).

### 5.3. Synthesis and Analytical Characterization of ind-imi-PtBr(ethane-1,2-diamine) (5c)

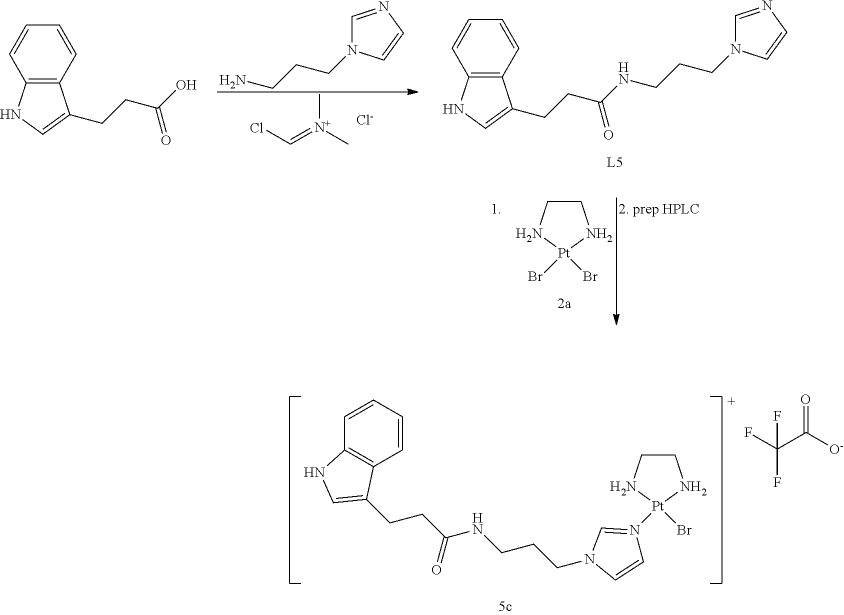

### 5.3.1. Synthesis of the Ligand N-(3-(1H-imidazol-1-yl)propyl)-3-(1H-indol-3-yl)propanamide (ind-imi, L5)

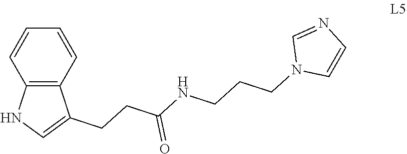 [SMILES: O=C(CCc1c[nH]c2ccccc12)NCCCn1ccnc1]

3-(1H-Indol-3-yl)propanoic acid (398 mg, 2.0 mmol, 1.0 eq.) was dissolved in dry DMF (5 mL) and N-(chloromethylene)-N-methylmethanaminium chloride (267 mg, 2.0 mmol, 1.0 eq.) was added at room temperature and stirred for 30 min at 40° C. Then, after cooling to room temperature and stirring for 1.5 h, 3-(1H-imidazol-1-yl)propan-1-amine (243 μL, 2.0 mmol, 1.0 eq.) was added, followed by the addition of DIPEA (1.7 mL, 10.0 mmol, 5.0 eq.). After stirring at room temperature for 22 h, the mixture was diluted with water, extracted with DCM, and after removal of solvents under reduced pressure the residue was absorbed on Celite and purified chromatographically on silica (eluent: DCM/MeOH/NH_{3 aq.}=100:1:1 to 100:2:1 to 100:3:1 to 100:4:1) as an. After drying, a yellow oil (383 mg, 65% yield) was obtained.

HRMS (ESI⁺) C₁₇H₂₁N₄O [M+H]⁺ calc 297.1710, found 297.1697

¹H NMR (400 MHz, DMSO-d₆): δ 10.77 (s, 1H, NH), 7.92-7.86 (m, 1H, NH), 7.56 (s, 1H), 7.55-7.51 (m, 1H), 7.34-7.30 (m, 1H), 7.12 (s, 1H), 7.11-7.08 (m, 1H), 7.08-7.02 (m, 1H), 6.99-6.94 (m, 1H), 6.87 (s, 1H), 3.85 (t, J=6.9 Hz, 2H), 3.04-2.96 (m, 2H), 2.93 (t, J=7.6 Hz, 2H), 2.45 (t, J=7.6 Hz, 2H), 1.77 (quint, J=6.8 Hz, 2H).

HPLC (Grace Alltima C18 5 μm column, 25×4.6 mm) indicated that the product was 100% pure (retention time 14.5 min; gradient: 5 to 50% MeCN/0.1% TFA in water/0.1% TFA in 18 min measured at a wavelength of 273 nm).

### 5.3.2. Synthesis of the Complex [ind-imi-PtBr(ethane-1,2-diamine)]⁺ TFA⁻ (5c)

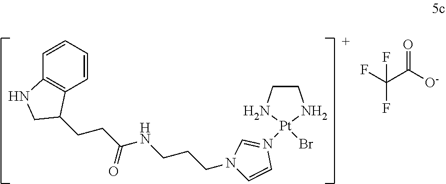

N-(3-(1H-Imidazol-1-yl)propyl)-3-(1H-indol-3-yl)propanamide (L5) (ind-imi; 14.8 mg, 50 μmol, 1.0 eq.) and PtBr₂(ethane-1,2-diamine) (2a) (31.1 mg, 75 μmol, 1.5 eq.) were dissolved in dry DMF (500 μL) under argon atmosphere. Triethylamine (10.5 μL, 75 μmol, 1.5 eq.) was added and the course of the reaction was followed by HPLC. The reaction mixture was stirred at 60° C. for 20 h, then the temperature was increased to 70° C. and the reaction mixture was stirred for an additional 20 h. At this moment, the reaction mixture contained 53.9% of the desired product and 5.2% starting material.

The reaction mixture was diluted with water/MeOH (4:1, 2.5 mL) and filtered through a 0.2 μm syringe filter. Purification was performed by preparative reverse-phase HPLC (Grace Alltima C18 5 μm column, 22×250 mm; gradient: 35 to 70% MeOH/0.1% TFA in water/0.1% TFA in 36 min). Product fractions were lyophilized resulting in a colorless solid (7.7 mg, 20.7% yield).

HRMS (ESI⁺) C₁₉H₂₈⁷⁹BrN₆O¹⁹⁵Pt[M]⁺ calc 630.1151, found 630.1140

HPLC (Grace Alltima C18 5 μm column, 25×4.6 mm) indicated that the product was 98.8% pure (retention time 17.2 min; gradient: 5 to 50% MeCN/0.1% TFA in water/0.1% TFA in 20 min measured at a wavelength of 223 nm).

### 5.4. Synthesis and Analytical Characterization of [(Fe)DFO-pip-PtBr(ethane-1,2-diamine)]⁺ TFA⁻ (5d)

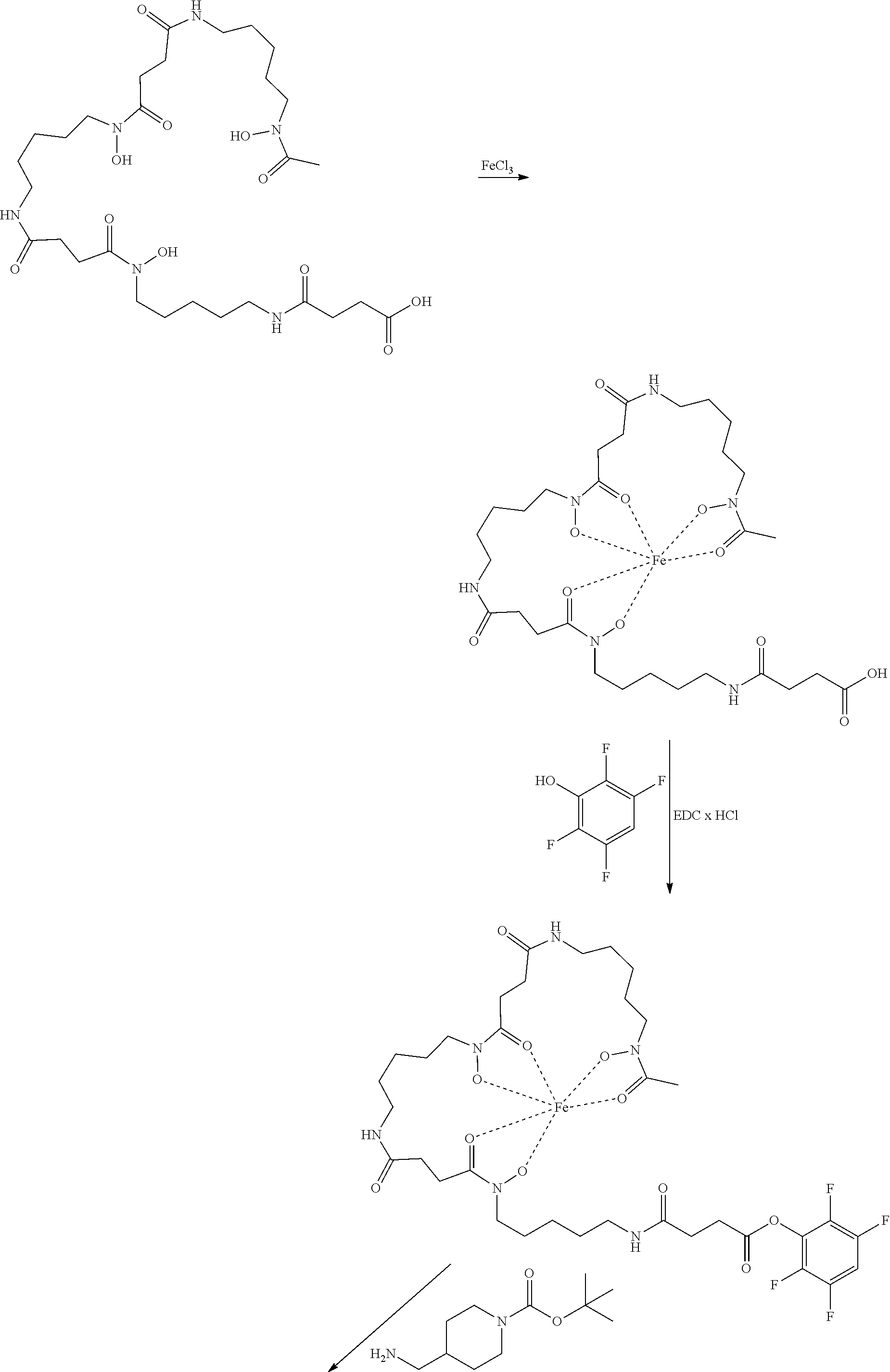

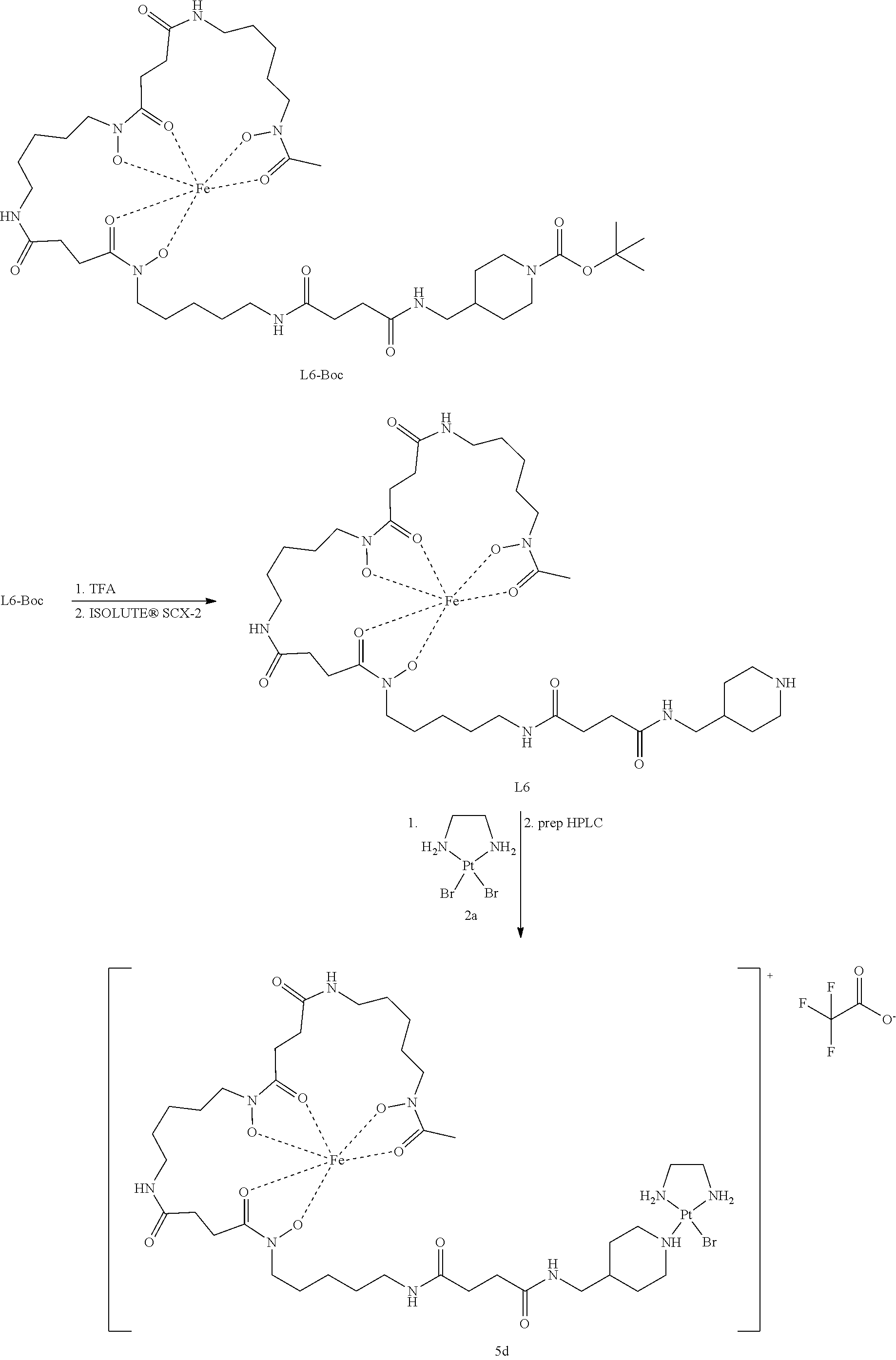

### 5.4.1. Synthesis of (Fe)DFO-suc

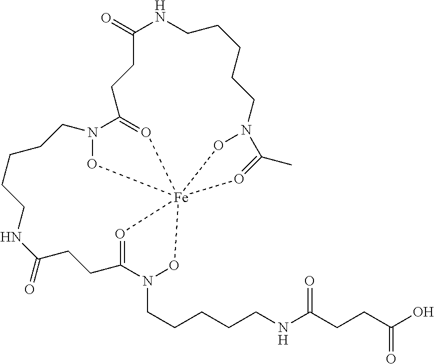

The procedure was adapted from Vugts et al., Bioconjugate Chem. 2011, 22, 2072-2081.

A solution of FeCl₃ (400 mg/mL in 0.5 M HCl) was prepared and 90 μL of this solution was added dropwise to a mixture of N-succinyl Desferal (DFO-suc, 120 mg, 182 μmol) in 0.1 M Na₂CO₃ (2.64 mL) and 0.9% NaCl (2.31 mL). The resulting mixture was stirred at room temperature for 10 min. The reaction mixture was used in the next step without further workup or purification.

### 5.4.2. Synthesis of (Fe)DFO-suc-TFP

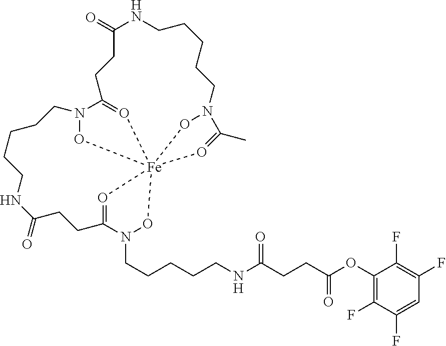

The procedure was adapted from Vugts et al., Bioconjugate Chem. 2011, 22, 2072-2081.

To the reaction mixture containing (Fe)DFO-suc (130 mg, 182 μmol) were added 0.9% NaCl (5 mL), MeCN (1.8 mL) and 2,3,5,6-tetrafluorophenol (290 mg, 1.75 mmol) in MeCN (200 μL). Next, EDC×HCl (550 mg, 2.87 mmol) was added and the mixture was stirred for 15 min. Subsequently, a second portion of EDC×HCl (500 mg, 2.61 mmol) was added and the mixture was stirred for another 15 min. The reaction mixture was divided into two equal batches and poured into 0.9% NaCl (30 mL each) and the resulting mixtures were trapped on two activated double Sep-Pak C18 Plus columns. These two double Sep-Pak C18 Plus columns were washed with water (3×20 mL each), and the product was eluted with 2×1.5 mL MeCN. Thus, two product batches were collected, each containing the product in ˜3 mL of solvents.

HPLC (Grace Alltima C18 5 μm column, 25×4.6 mm) indicated that batch 1 was 94.8% pure and batch 2 was 95.2% pure (retention time 20.4 min; gradient: 5 to 50% MeCN/0.1% TFA in water/0.1% TFA in 20 min measured at a wavelength of 430 nm). It was assumed that the yield was ˜80% (based on the results obtained by Vugts et al., Bioconjugate Chem. 2011, 22, 2072-2081). The two solutions containing product were used in the next step without further workup or purification.

### 5.4.3. Synthesis of (Fe)DFO-suc-pip-Boc (L6-Boc)

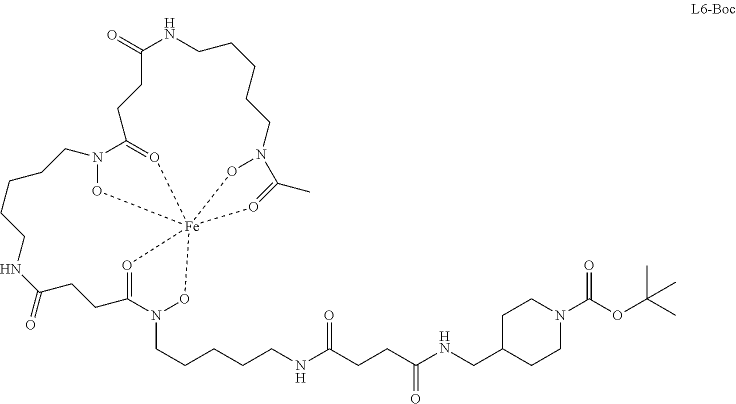

tert-Butyl 4-(aminomethyl)piperidine-1-carboxylate (23.5 mg, 110 μmol) was suspended in MeCN (300 μL) and the mixture was added to (Fe)DFO-suc-TFP (batch 2; ˜63 mg, 73 μmol in 3 mL MeCN; 95.2% purity). Subsequently, DIPEA (25.5 μL, 146 μmol) was added to the reaction mixture, which was stirred at room temperature. HPLC (Grace Alltima C18 5 μm column, 25×4.6 mm) indicated that the product was >95% pure after stirring for 75 min (retention time 18.4 min; gradient: 5 to 50% MeCN/0.1% TFA in water/0.1% TFA in 20 min measured at a wavelength of 430 nm). The reaction mixture containing L6-Boc was evaporated and used in the next step without further purification.

### 5.4.4. Synthesis of the Ligand (Fe)DFO-suc-pip (L6)

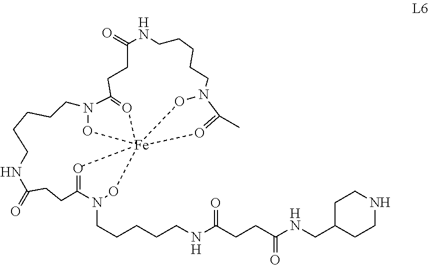

The crude material L6-Boc (˜67 mg, 73 μmol) was dissolved in DCM (3 mL), and TFA (3 mL) was added. The resulting mixture was stirred for 1.5 h at room temperature, concentrated, and the resulting residue was dissolved in MeOH. This dissolved material was loaded on an ISOLUTE® SCX-2 column that was activated with DCM. The column was washed with MeOH, and subsequently with 0.25 M NH_{3 (aq)} in MeOH. The product was eluted with 1 M NH_{3 (aq)} in MeOH and subsequently with 7 M NH_{3 (aq)} in MeOH. The solvents were evaporated and the product was dissolved in water and lyophilized to afford a red solid (40.1 mg, 50.0 μmol, ˜55% over four steps from DFO-suc).

HRMS (ESI⁺) C₃₅H₆₂FeN₈O₁₀ [M+H]⁺ calc 810.3933, found 810.3928

HPLC (Grace Alltima C18 5 μm column, 25×4.6 mm) indicated that the product was 97.5% pure (retention time 11.8 min; gradient: 5 to 50% MeCN/0.1% TFA in water/0.1% TFA in 20 min measured at a wavelength of 430 nm).

### 5.4.5. Synthesis of the Complex [(Fe)DFO-pip-PtBr(ethane-1,2-diamine)]⁺ TFA⁻ (5d)

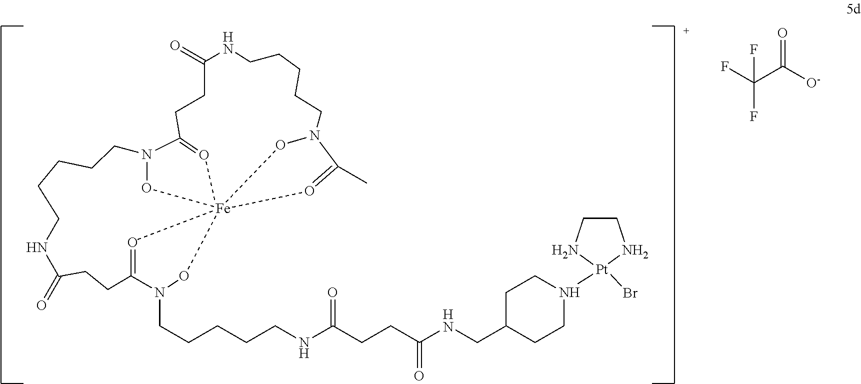

To an HPLC vial charged with L6 (16 mg, 20 μmol) were added DMF (200 μL), PtBr₂(ethane-1,2-diamine) (12.3 mg, 30 μmol), and TEA (4.13 μL, 30 μmol). The resulting mixture was shaken for 24 h at 60° C. The reaction mixture was diluted with water/MeOH (7:3, 3 mL) and filtered through a 0.2 μm syringe filter. Purification was performed by preparative reverse-phase HPLC (Grace Alltima C18 5 μm column, 22×250 mm; gradient: 30 to 50% MeOH/0.1% TFA in water/0.1% TFA in 36 min). Product fractions were collected and concentrated to −2/3 of the initial volume. Water (˜2 mL) was added and the mixture was lyophilized resulting in a red solid (14 mg, 56.3% yield). The product was dissolved in an aqueous 20 mM NaBr solution and stored as a 5 mM solution.

HRMS (ESI⁺) C₃₇H₁₆₉Fe⁷⁹BrN₁₀O₁₀¹⁹⁵Pt[M]⁺ calc 1143.3379, found 1143.3258

HPLC (Grace Alltima C18 5 μm column, 25×4.6 mm) indicated that the product was 95.6% pure (retention time 13.1 min; gradient: 5 to 50% MeCN/0.1% TFA in water/0.1% TFA in 20 min measured at a wavelength of 430 nm).

### Example 6: Examples of Iodido Lx-“Semi-Final Products” I-Lx-PFM (Iodido SFMs)

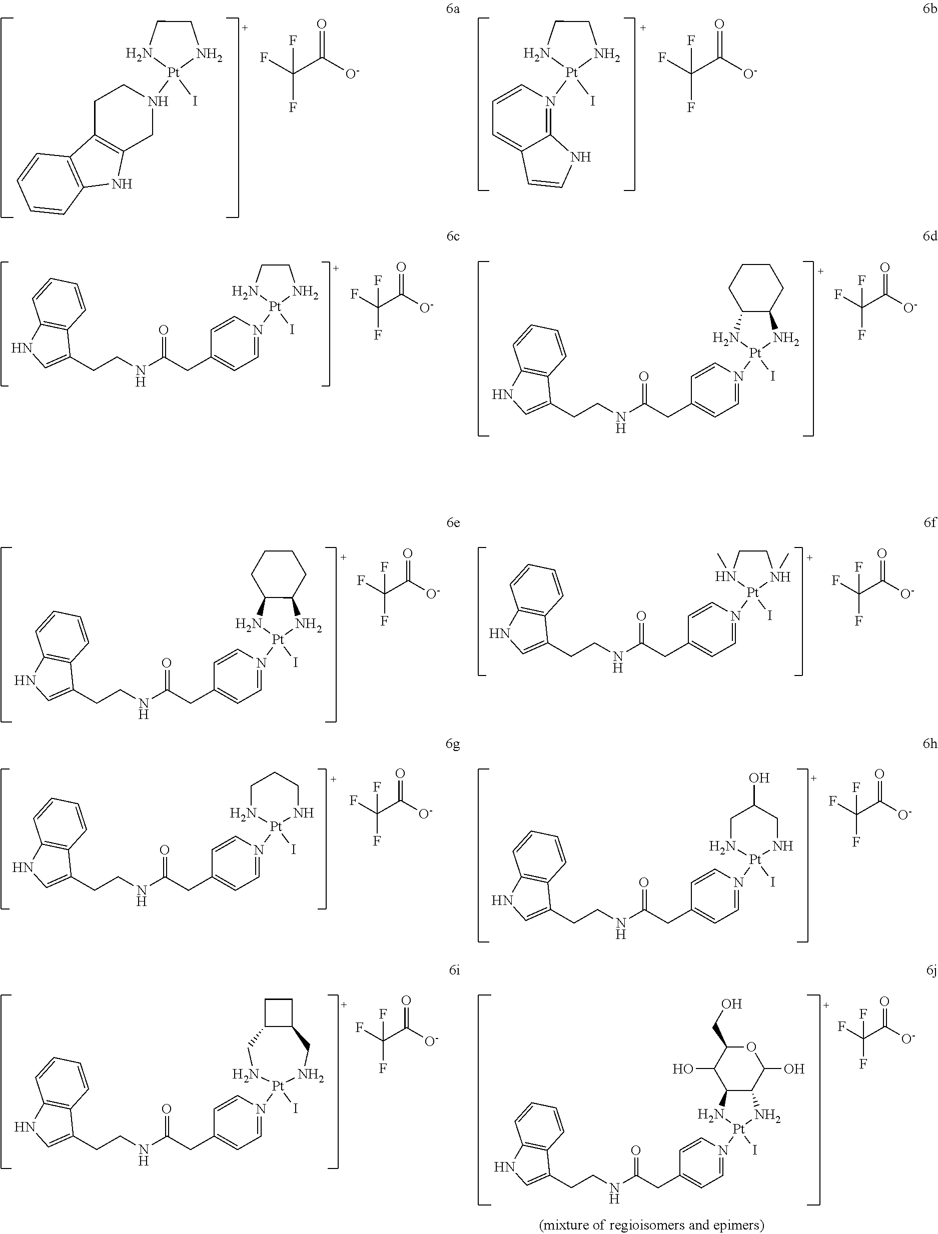

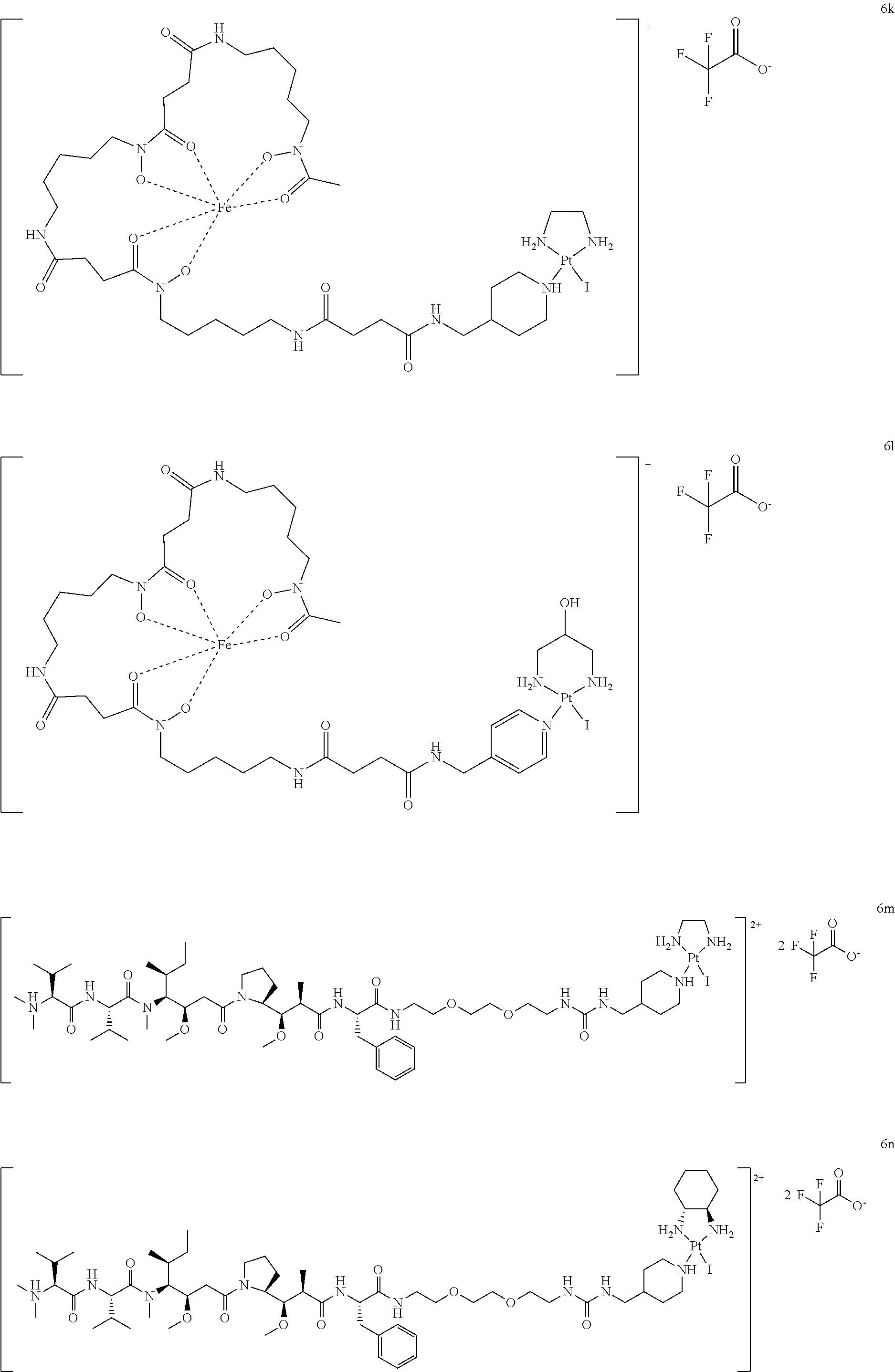

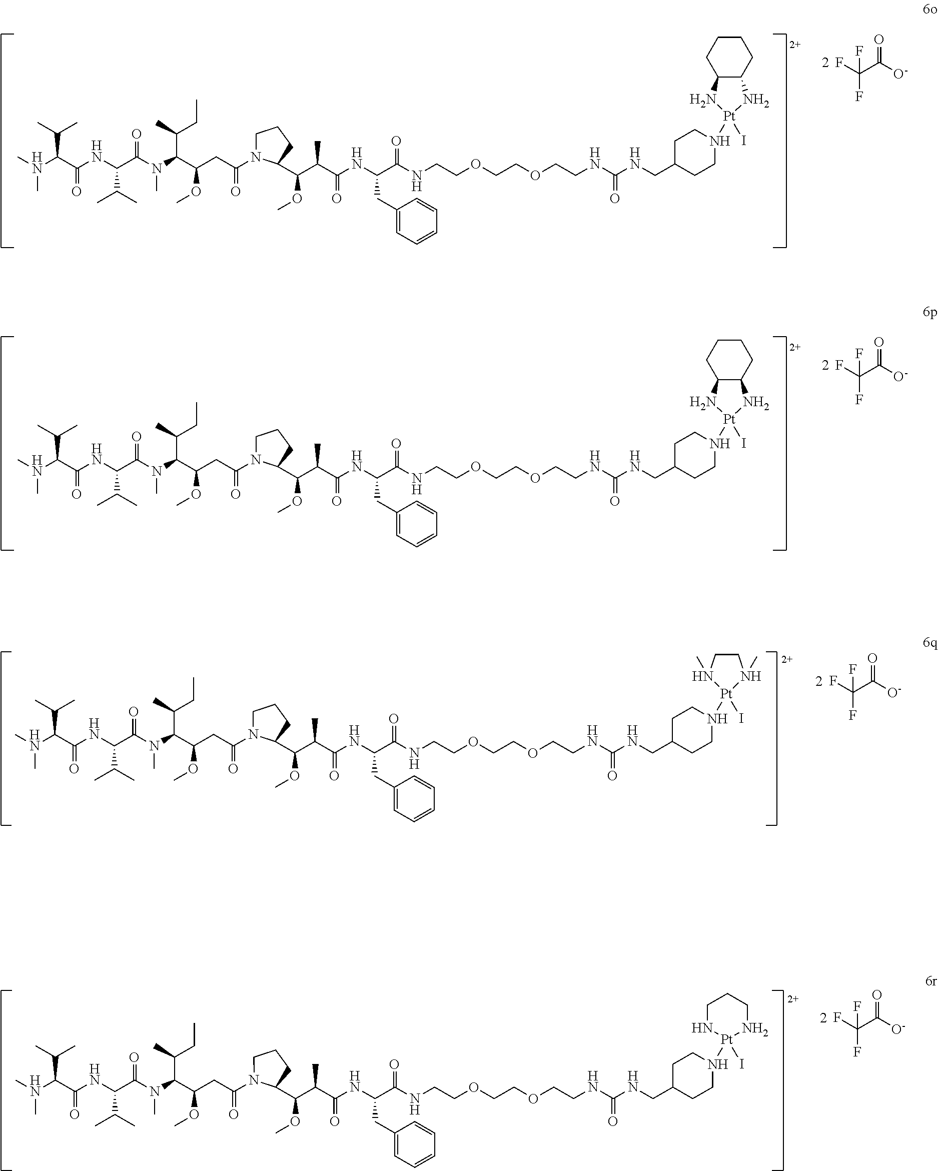

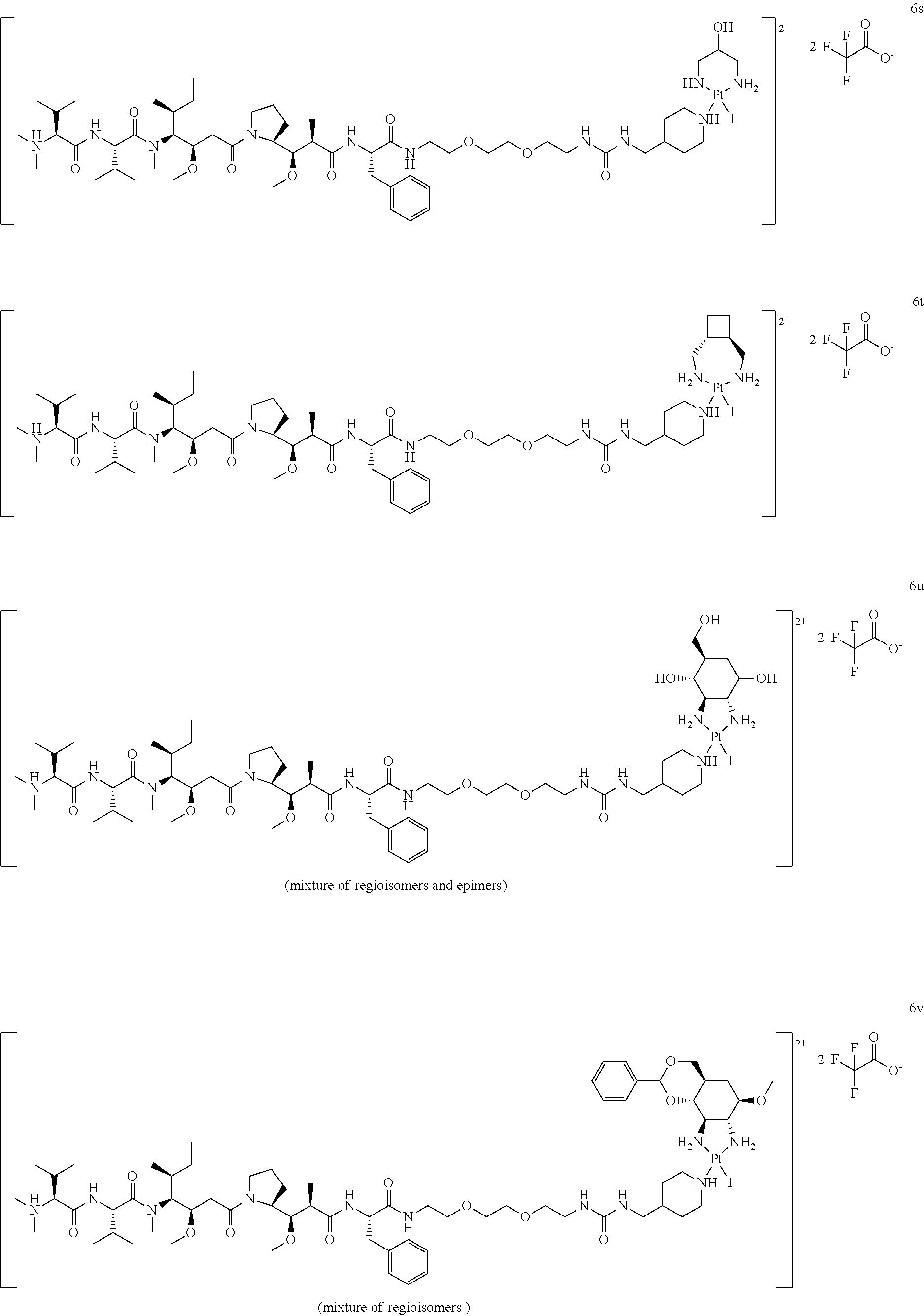

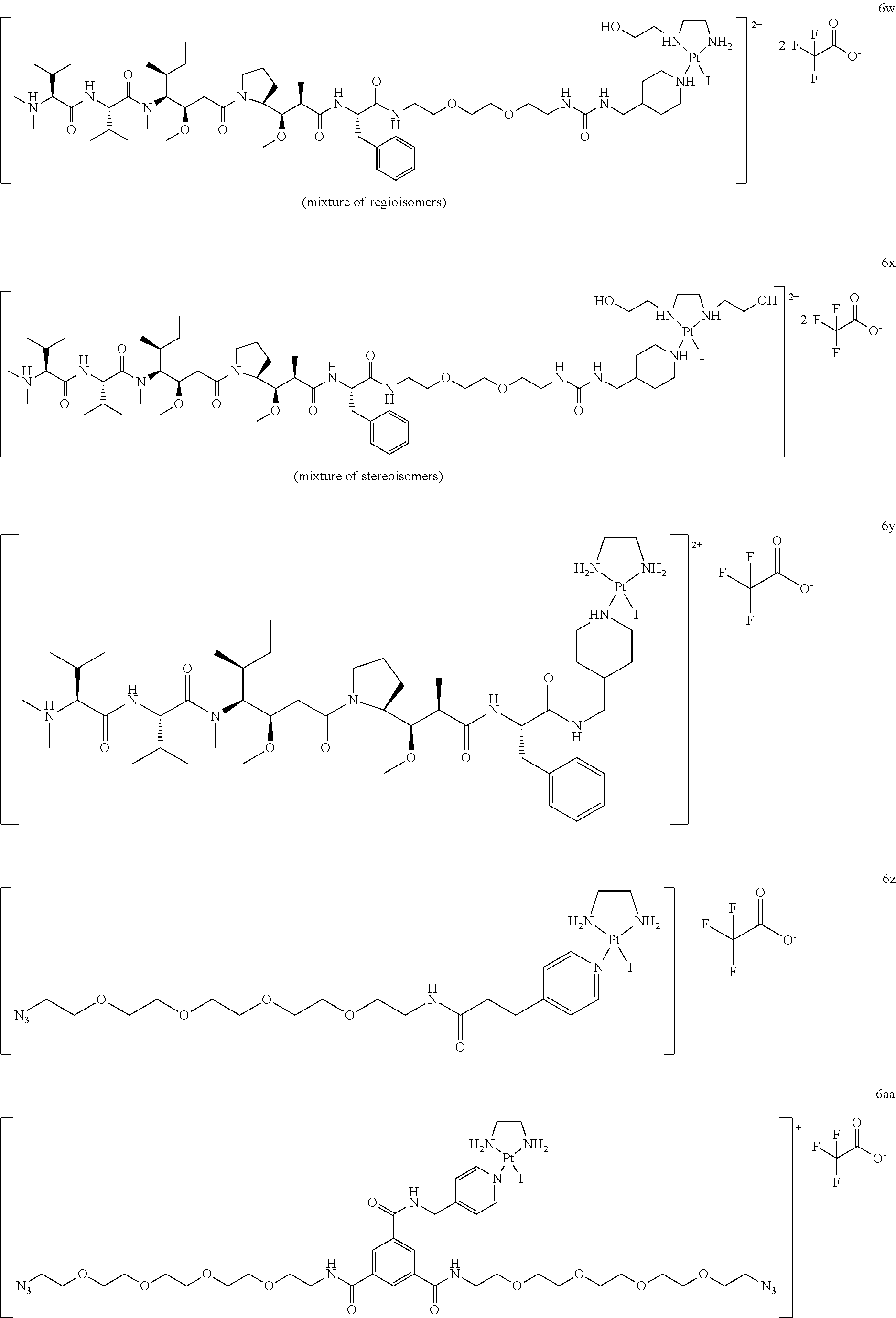

### 6.1. Synthesis and Analytical Characterization of [noreleagnine-Pt(ethane-1,2-diamine)I]⁺ TFA⁻ (6a)

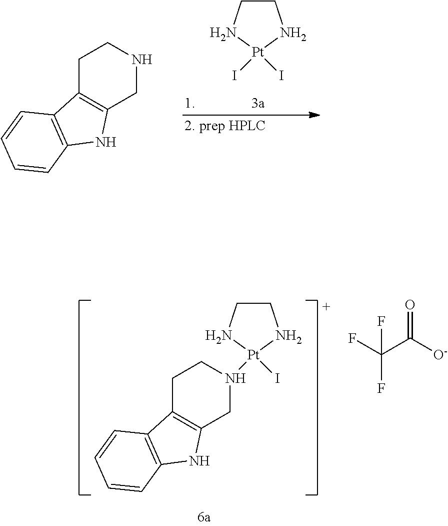

2,3,4,9-Tetrahydro-1H-pyrido[3,4-b]indole (noreleagnine; 9.1 mg, 50 μmol, 1.0 eq.) and Pt(ethane-1,2-diamine)I₂ (3a) (25.4 mg, 50 μmol, 1.0 eq.) were dissolved in dry DMF (333 μmol). Triethylamine (6.98 μL, 50 μmol, 1.0 eq.) was added and the course of the reaction was followed by HPLC. The reaction mixture was stirred at 60° C. for 24 h. At this moment, the reaction mixture contained 84.1% of the desired product and 4.4% of starting material (retention time 14.4 min).

The reaction mixture was diluted with water/MeOH (19:1, 2.5 mL) and filtered through a 0.2 μm syringe filter. Purification was performed by preparative reverse-phase HPLC (Grace Alltima C18 5 μm column, 22×250 mm; gradient: 20 to 100% MeOH/0.1% TFA in water/0.1% TFA in 36 min). Product fractions were lyophilized resulting in a colorless solid (19.9 mg, 59.6% yield).

HRMS (ESI⁺) C₁₃H₂₀IN₄¹⁹⁵Pt[M]⁺ calc 554.0376, found 554.0369

HPLC (Grace Alltima C18 5 μm column, 25×4.6 mm) indicated that the product was 97.9% pure (retention time 19.9 min; gradient: 5 to 50% MeCN/0.1% TFA in water/0.1% TFA in 18 min measured at a wavelength of 273 nm).

### 6.2. Synthesis and Analytical Characterization of [7-azaindole-Pt(ethane-1,2-diamine)I]⁺ TFA⁻ (6b)

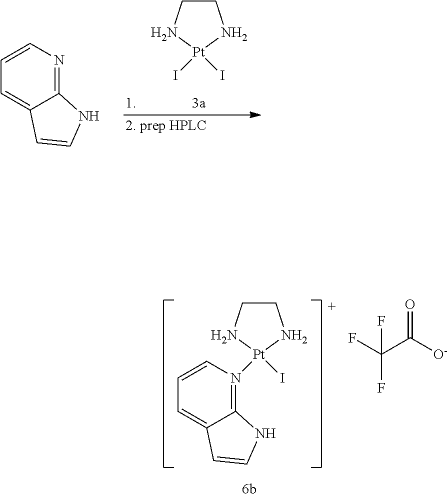

1H-Pyrrolo[2,3-b]pyridine (7-azaindole; 6.0 mg, 50 μmol, 1.0 eq.) and Pt(ethane-1,2-diamine)I₂ (3a) (25.4 mg, 50 μmol, 1.0 eq.) were dissolved in dry DMF (333 μmol). Triethylamine (6.98 μL, 50 μmol, 1.0 eq.) was added and the course of the reaction was followed by HPLC. The reaction mixture was stirred at 60° C. for 24 h. At this moment, the reaction mixture contained 72.8% of the desired product and 26.9% of starting material (retention time 4.5 min).

The reaction mixture was diluted with water/MeOH (19:1, 2.5 mL) and filtered through a 0.2 μm syringe filter. Purification was performed by preparative reverse-phase HPLC (Grace Alltima C18 5 μm column, 22×250 mm; gradient: 20 to 100% MeOH/0.1% TFA in water/0.1% TFA in 36 min). Product fractions were lyophilized resulting in a colorless solid (12.2 mg, 39.8% yield).

HRMS (ESI⁺) C₉H₁₄IN₄¹⁹⁵Pt[M]⁺ calc 499.9906, found 499.9910

HPLC (Grace Alltima C18 5 μm column, 25×4.6 mm) indicated that the product was 99.5% pure (retention time 14.8 min; gradient: 5 to 50% MeCN/0.1% TFA in water/0.1% TFA in 18 min measured at a wavelength of 273 nm).

### 6.3. Synthesis and Analytical Characterization of [ind-py-Pt(ethane-1,2-diamine)I]⁺ TFA⁻ (6c)

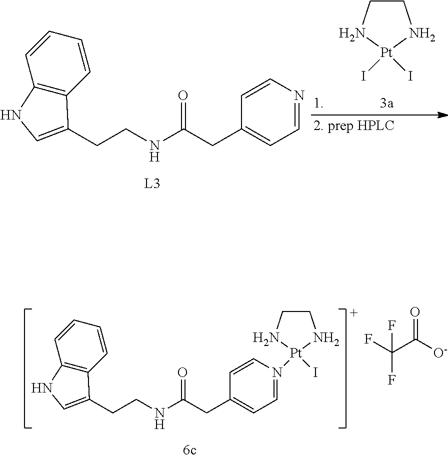

N-(2-(1H-Indol-3-yl)ethyl)-2-(pyridin-4-yl)acetamide (L3) (ind-py; 14.0 mg, 50 μmol, 1.0 eq.) and Pt(ethane-1,2-diamine)I₂ (3a) (25.4 mg, 50 μmol, 1.0 eq.) were dissolved in dry DMF (333 μL) under argon atmosphere. Triethylamine (6.98 μL, 50 μmol, 1.0 eq.) was added and the course of the reaction was followed by HPLC. The reaction mixture was stirred at 60° C. for 23 h. At this moment, the reaction mixture contained 95.0% product and 5.0% starting material.

The reaction mixture was diluted with water/MeOH (4:1, 2.5 mL) and filtered through a 0.2 μm syringe filter. Purification was performed by preparative reverse-phase HPLC (Grace Alltima C18 5 μm column, 22×250 mm; gradient: 20 to 100% MeOH/0.1% TFA in water/0.1% TFA in 36 min). Product fractions were lyophilized resulting in a colorless solid (25.2 mg, 65.1% yield).

HRMS (ESI⁺) C₁₉H₂₅IN₅O¹⁹⁵Pt[M]⁺ calc 661.0747, found 661.0731

HPLC (Grace Alltima C18 5 μm column, 25×4.6 mm) indicated that the product was 99.6% pure (retention time 18.8 min; gradient: 5 to 50% MeCN/0.1% TFA in water/0.1% TFA in 18 min measured at a wavelength of 273 nm).

### 6.4. Synthesis and Analytical Characterization of [ind-py-Pt(((1R,2R)-(−)-1,2-diaminocyclohexane))I]⁺ TFA⁻ (6d)

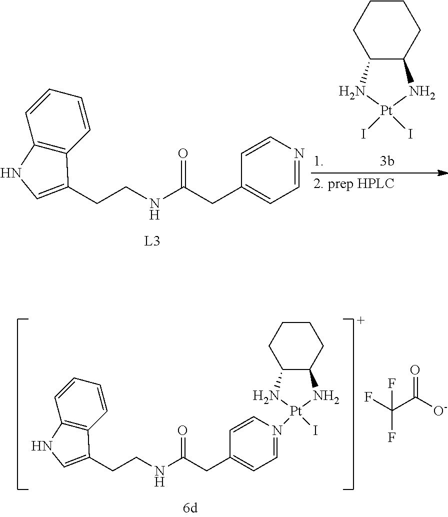

N-(2-(1H-Indol-3-yl)ethyl)-2-(pyridin-4-yl)acetamide (L3) (ind-py; 14.0 mg, 50 μmol, 1.0 eq.) and Pt(((1R,2R)-(−)-1,2-diaminocyclohexane))I₂ (3b) (42.2 mg, 75 μmol, 1.5 eq.) were dissolved in dry DMF (333 μL) under argon atmosphere. Triethylamine (10.46 μL, 75 μmol, 1.5 eq.) was added and the course of the reaction was followed by HPLC. The reaction mixture was stirred at 40° C. for 68 h and then at 50° C. for 24 h. At this moment, the reaction mixture contained 90.2% product and 4.0% starting material.

The reaction mixture was diluted with water/MeOH (4:1, 2.5 mL) and filtered through a 0.2 μm syringe filter. Purification was performed by preparative reverse-phase HPLC (Grace Alltima C18 5 μm column, 22×250 mm; gradient: 35 to 70% MeOH/0.1% TFA in water/0.1% TFA in 36 min). Product fractions were lyophilized resulting in a colorless solid (19.7 mg, 47.6% yield).

HRMS (ESI⁺) C₂₃H₃₁IN₅O¹⁹⁵Pt[M]⁺ calc 715.1216, found 715.1194

HPLC (Grace Alltima C18 5 μm column, 25×4.6 mm) indicated that the product was 99.6% pure (retention time 12.5 min; gradient: 20 to 100% MeCN/0.1% TFA in water/0.1% TFA in 20 min measured at a wavelength of 273 nm).

### 6.5. Synthesis and Analytical Characterization of [ind-py-Pt(cis-1,2-diaminocyclohexane)I]⁺ TFA⁻ (6e)

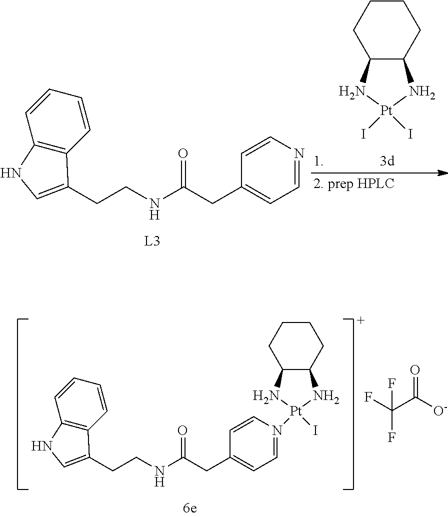

N-(2-(1H-Indol-3-yl)ethyl)-2-(pyridin-4-yl)acetamide (L3) (ind-py; 14.0 mg, 50 μmol, 1.0 eq.) and Pt(cis-1,2-diaminocyclohexane)I₂ (3d) (42.4 mg, 75 μmol, 1.5 eq.) were dissolved in dry DMF (333 μL) under argon atmosphere. Triethylamine (10.45 μL, 75 μmol, 1.5 eq.) was added and the course of the reaction was followed by HPLC. The reaction mixture was stirred at 40° C. for 19 h. At this moment, the reaction mixture contained 88.4% product and 6.0% starting material.

The reaction mixture was diluted with water/MeOH (4:1, 2.5 mL) and filtered through a 0.2 μm syringe filter. Purification was performed by preparative reverse-phase HPLC (Grace Alltima C18 5 μm column, 22×250 mm; gradient: 35 to 70% MeOH/0.1% TFA in water/0.1% TFA in 36 min). Product fractions were lyophilized resulting in a colorless solid (15.4 mg, 37.2% yield).

HRMS (ESI⁺) C₂₃H₃₁IN₅O¹⁹⁵Pt[M]⁺ calc 715.1216, found 715.1195

HPLC (Grace Alltima C18 5 μm column, 25×4.6 mm) indicated that the product was 99.6% pure (retention time 12.3 min; gradient: 20 to 100% MeCN/0.1% TFA in water/0.1% TFA in 20 min measured at a wavelength of 273 nm).

### 6.6. Synthesis and Analytical Characterization of [ind-py-Pt(N¹,N²-dimethylethane-1,2-diamine)I]⁺ TFA⁻ (6f)

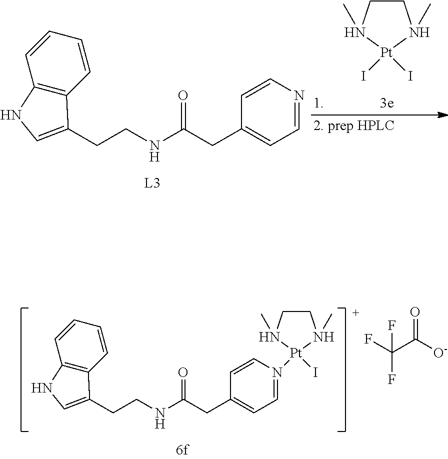

N-(2-(1H-Indol-3-yl)ethyl)-2-(pyridin-4-yl)acetamide (L3) (ind-py; 14.0 mg, 50 μmol, 1.0 eq.) and Pt(N¹,N²-dimethylethane-1,2-diamine)I₂ (3e) (40.3 mg, 75 μmol, 1.5 eq.) were dissolved in dry DMF (333 μL) under argon atmosphere. Triethylamine (10.45 μL, 75 μmol, 1.5 eq.) was added and the course of the reaction was followed by HPLC. The reaction mixture was stirred at 40° C. for 20 h. At this moment, the reaction mixture contained 89.9% product and 11.5% starting material.

The reaction mixture was diluted with water/MeOH (4:1, 2.5 mL) and filtered through a 0.2 μm syringe filter. Purification was performed by preparative reverse-phase HPLC (Grace Alltima C18 5 μm column, 22×250 mm; gradient: 35 to 70% MeOH/0.1% TFA in water/0.1% TFA in 36 min). Product fractions were lyophilized resulting in a colorless solid (12.4 mg, 30.9% yield).

HRMS (ESI⁺) C₂₁H₂₉IN₅O¹⁹⁵Pt[M]⁺ calc 689.1060, found 689.1043

HPLC (Grace Alltima C18 5 μm column, 25×4.6 mm) indicated that the product was 100% pure (retention time 11.6 min; gradient: 20 to 100% MeCN/0.1% TFA in water/0.1% TFA in 20 min measured at a wavelength of 223 nm).

### 6.7. Synthesis and Analytical Characterization of [ind-py-PtI(propane-1,3-diamine)]⁺ TFA⁻ (6g)

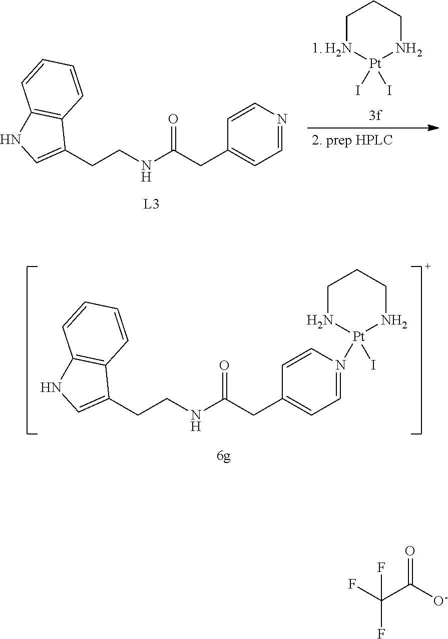

N-(2-(1H-Indol-3-yl)ethyl)-2-(pyridin-4-yl)acetamide (L3) (ind-py; 14.0 mg, 50 μmol, 1.0 eq.) and PtI₂(propane-1,3-diamine) (3f) (39.2 mg, 75 μmol, 1.5 eq.) were dissolved in dry DMF (333 μL) under argon atmosphere. Triethylamine (10.45 μL, 75 μmol, 1.5 eq.) was added and the course of the reaction was followed by HPLC. The reaction mixture was stirred at 25° C. for 16.5 h, then continued at 30° C. for 5 h, at 40° C. for 18 h, and finally at 50° C. for 5 h. At this moment, the reaction mixture contained 97.3% product and 2.7% starting material.

The reaction mixture was diluted with water/MeOH (4:1, 2.5 mL) and filtered through a 0.2 μm syringe filter. Purification was performed by preparative reverse-phase HPLC (Grace Alltima C18 5 μm column, 22×250 mm; gradient: 35 to 70% MeOH/0.1% TFA in water/0.1% TFA in 36 min). Product fractions were lyophilized resulting in a colorless solid (5.2 mg, 13.2% yield).

HRMS (ESI⁺) C₂₀H₂₇IN₅O¹⁹⁵Pt[M]⁺ calc 675.0903, found 675.0985

HPLC (Grace Alltima C18 5 μm column, 25×4.6 mm) indicated that the product was 97.9% pure (retention time 19.6 min; gradient: 5 to 50% MeCN/0.1% TFA in water/0.1% TFA in 18 min measured at a wavelength of 223 nm).

### 6.8. Synthesis and Analytical Characterization of [ind-py-Pt(1,3-diaminopropan-2-ol)I]⁺ TFA⁻ (6h)

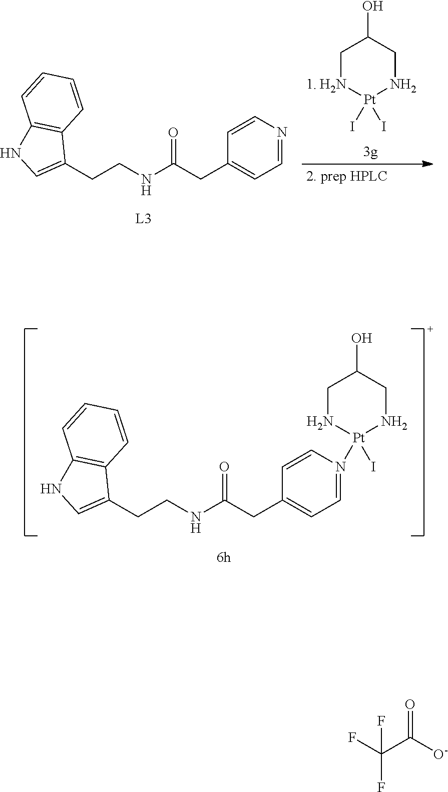

N-(2-(1H-Indol-3-yl)ethyl)-2-(pyridin-4-yl)acetamide (L3) (ind-py; 14.0 mg, 50 μmol, 1.0 eq.) and Pt(1,3-diaminopropan-2-ol)I₂ (3g) (40.4 mg, 75 μmol, 1.5 eq.) were dissolved in dry DMF (333 μL) under argon atmosphere. Triethylamine (10.45 μL, 75 μmol, 1.5 eq.) was added and the course of the reaction was followed by HPLC. The reaction mixture was stirred at 25° C. for 16.5 h, then continued at 30° C. for 5 h, at 40° C. for 18 h, and finally at 50° C. for 5 h. At this moment, the reaction mixture contained 93.4% product and 2.1% starting material.

The reaction mixture was diluted with water/MeOH (4:1, 2.5 mL) and filtered through a 0.2 μm syringe filter. Purification was performed by preparative reverse-phase HPLC (Grace Alltima C18 5 μm column, 22×250 mm; gradient: 35 to 70% MeOH/0.1% TFA in water/0.1% TFA in 36 min). Product fractions were lyophilized resulting in a colorless solid (16.1 mg, 40.0% yield).

HRMS (ESI⁺) C₂₀H₂₇IN₅O₂¹⁹⁵Pt[M]⁺ calc 691.0852, found 691.0960

HPLC (Grace Alltima C18 5 μm column, 25×4.6 mm) indicated that the product was 97.9% pure (retention time 18.7 min; gradient: 5 to 50% MeCN/0.1% TFA in water/0.1% TFA in 18 min measured at a wavelength of 223 nm).

### 6.9. Synthesis and Analytical Characterization of [ind-py-Pt(((1R,2R)-cyclobutane-1,2-diyl)dimethanamine)I]⁺ TFA⁻ (6i)

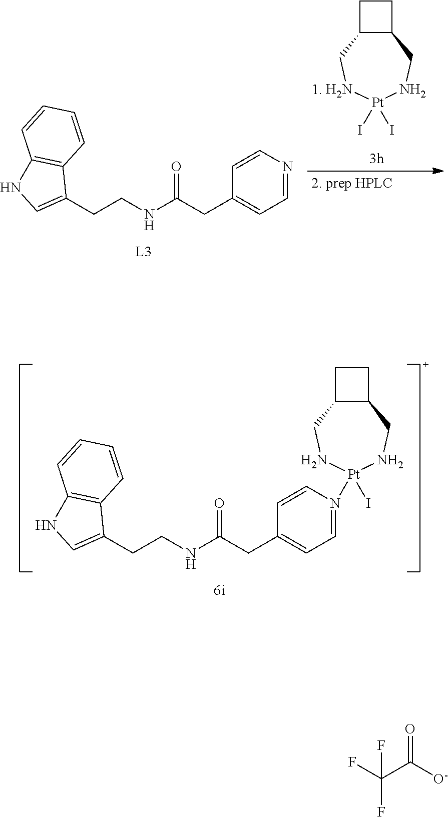

N-(2-(1H-Indol-3-yl)ethyl)-2-(pyridin-4-yl)acetamide (L3) (ind-py; 14.0 mg, 50 μmol, 1.0 eq.) and Pt(((1R,2R)-cyclobutane-1,2-diyl)dimethanamine)I₂ (3h) (42.2 mg, 75 μmol, 1.5 eq.) were dissolved in dry DMF (333 μL) under argon atmosphere. Triethylamine (10.45 μL, 75 μmol, 1.5 eq.) was added and the course of the reaction was followed by HPLC. The reaction mixture was stirred at 40° C. for 20 h. At this moment, the reaction mixture contained 69.3% product and 17.0% starting material.

The reaction mixture was diluted with water/MeOH (4:1, 2.5 mL) and filtered through a 0.2 μm syringe filter. Purification was performed by preparative reverse-phase HPLC (Grace Alltima C18 5 μm column, 22×250 mm; gradient: 35 to 80% MeOH/0.1% TFA in water/0.1% TFA in 36 min). Product fractions were lyophilized resulting in a colorless solid (4.8 mg, 11.6% yield).

HRMS (ESI⁺) C₂₃H₃₁IN₅₀¹⁹⁵Pt[M]⁺ calc 715.1216, found 715.1198

HPLC (Grace Alltima C18 5 μm column, 25×4.6 mm) indicated that the product was 95.9% pure (retention time 13.2 min; gradient: 20 to 100% MeCN/0.1% TFA in water/0.1% TFA in 20 min measured at a wavelength of 273 nm).

### 6.10. Synthesis and Analytical Characterization of [ind-py-Pt((3R,4R,5S,6R)-3,4-diamino-6-(hydroxymethyl)tetrahydro-2H-pyran-2,5-diol)I]⁺ TFA⁻ (6j)

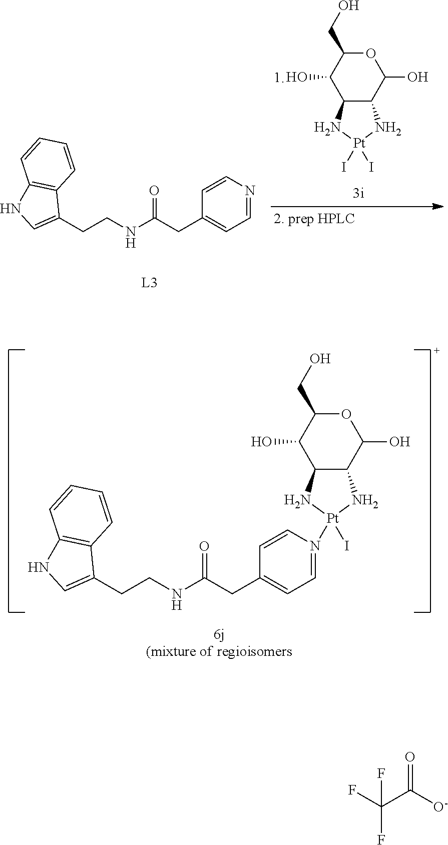

N-(2-(1H-Indol-3-yl)ethyl)-2-(pyridin-4-yl)acetamide (L3) (ind-py; 14.0 mg, 50 μmol, 1.0 eq.) and Pt((3R,4R,5S,6R)-3,4-diamino-6-(hydroxymethyl)tetrahydro-2H-pyran-2,5-diol)I₂ (3i) (47.0 mg, 75 μmol, 1.5 eq.) were dissolved in dry DMF (500 μL) under argon atmosphere. Triethylamine (10.45 μL, 75 μmol, 1.5 eq.) was added and the course of the reaction was followed by HPLC. The reaction mixture was stirred at 50° C. for 25 h. At this moment, the reaction mixture contained 82.6% product and 5.8% starting material.

The reaction mixture was diluted with 35% MeOH/water (2.0 mL) and filtered through a 0.2 μm syringe filter. Purification was performed by preparative reverse-phase HPLC (Grace Alltima C18 5 μm column, 22×250 mm; gradient: 35 to 70% MeOH/0.1% TFA in water/0.1% TFA in 36 min). Product fractions were lyophilized resulting in a beige solid (21.0 mg, 47.1% yield).

HRMS (ESI⁺) C₂₃H₃₁IN₅O₅¹⁹⁵Pt[M]⁺ calc 779.1013, found 779.1042

HPLC (Grace Alltima C18 5 μm column, 25×4.6 mm) indicated that the product was 99.2% pure (note: the product was obtained as a mixture of regioisomers and epimers, so that several peaks were observed; retention times 16.8-17.6 min; gradient: 5 to 50% MeCN/0.1% TFA in water/0.1% TFA in 18 min measured at a wavelength of 273 nm).

### 6.11. Synthesis and Analytical Characterization of the Complex [Pt((Fe)DFO-suc-pip)(ethane-1,2-diamine)I]⁺ TFA⁻ (6k)

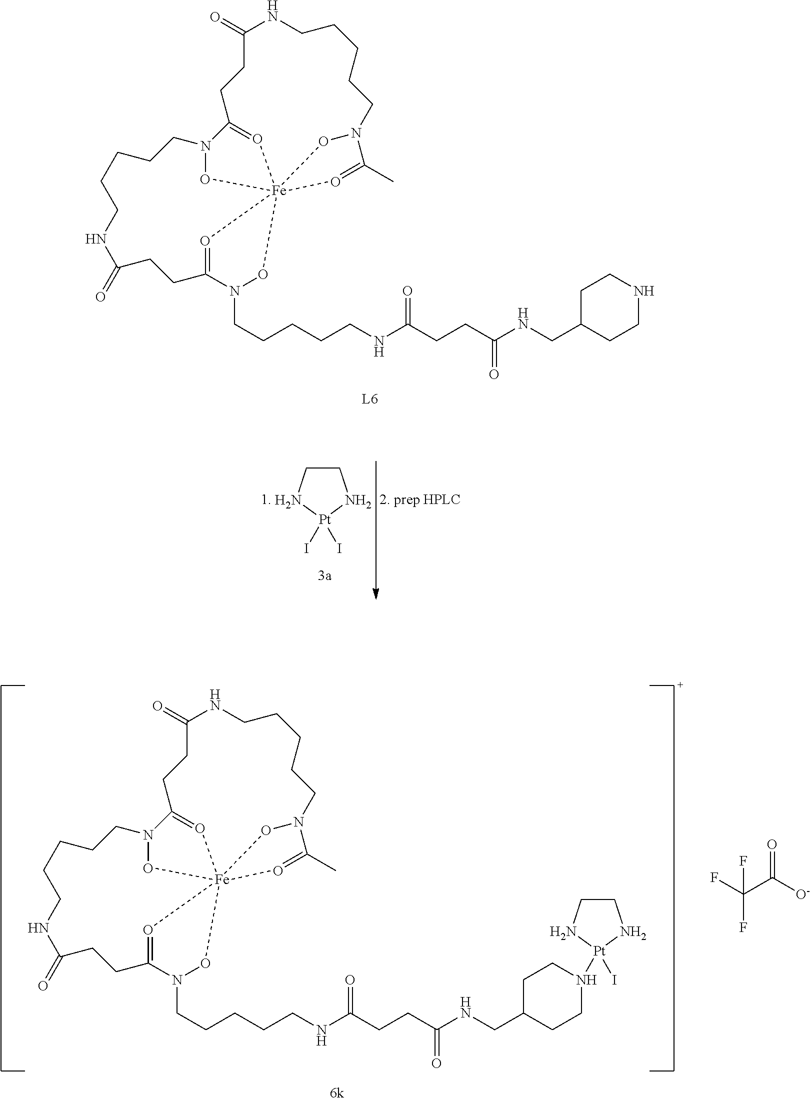

To an HPLC vial charged with (Fe)DFO-suc-pip (L6) (16 mg, 20 μmol, 1.0 eq.) were added DMF (200 μL), Pt(ethane-1,2-diamine)I₂ (3a) (15.1 mg, 30 μmol, 1.5 eq.), and TEA (4.13 μL, 30 μmol, 1.5 eq.). The resulting mixture was shaken for 20 h at 60° C. The reaction mixture was diluted with water/MeOH (7:3, 3 mL) and filtered through a 0.2 μm syringe filter. Purification was performed by preparative reverse-phase HPLC (Grace Alltima C18 5 μm column, 22×250 mm; gradient: 30 to 50% MeOH/0.1% TFA in water/0.1% TFA in 36 min). Product fractions were collected and reduced to ˜2/3 of the initial volume. Water (˜5 mL) was added and the mixture was lyophilized resulting in a red solid (11 mg, 42.7% yield). The product was dissolved in an aqueous 20 mM NaI solution and stored as a 5 mM solution.

HRMS (ESI⁺) C₃₇H₆₉FeIN₁₀O₁₀¹⁹⁵Pt[M]⁺ calc 1191.3235, found 1191.3412

HPLC (Grace Alltima C18 5 μm column, 25×4.6 mm) indicated that the product was 95.7% pure (retention time 13.8 min; gradient: 5 to 50% MeCN/0.1% TFA in water/0.1% TFA in 20 min measured at a wavelength of 430 nm).

### 6.12. Synthesis and Analytical Characterization of the Complex [(Fe)DFO-suc-py-Pt(1,3-diaminopropan-2-ol)I]⁺ TFA⁻ (6l)

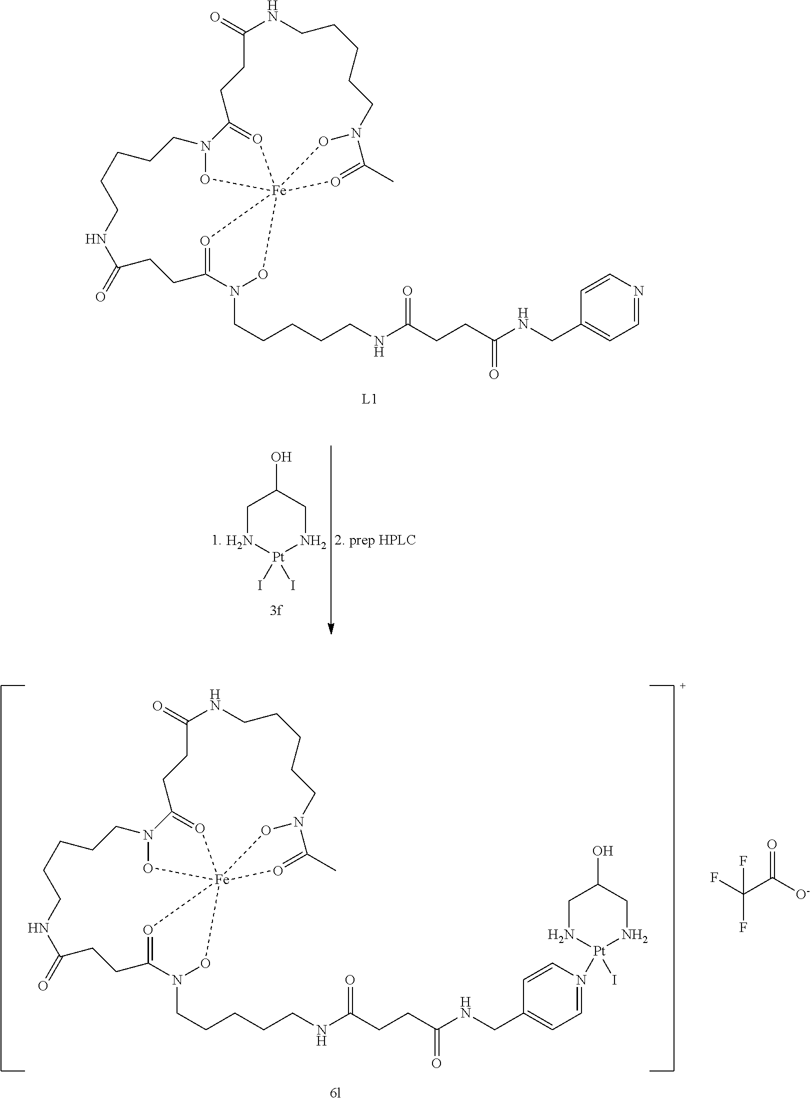

(Fe)DFO-suc-py (L1) (10.0 mg, 12 μmol, 1.0 eq.) and Pt(1,3-diaminopropan-2-ol)I₂ (3f) (26.4 mg, 48 μmol, 4 eq.) were dissolved in dry DMF (375 μL) under argon atmosphere. Triethylamine (6.92 μL, 48 μmol, 4 eq.) was added and the course of the reaction was followed by HPLC. The reaction mixture was stirred at 40° C. for 16 h. At this moment, the reaction mixture contained 81.0% product and no starting material.

The reaction mixture was diluted with water/MeOH (2:1, 2.5 mL) and filtered through a 0.2 μm syringe filter. Purification was performed by preparative reverse-phase HPLC (Grace Alltima C18 5 μm column, 22×250 mm; gradient: 30 to 55% MeOH/0.1% TFA in water/0.1% TFA in 36 min). Product fractions were lyophilized resulting in a colorless solid (7.6 mg, 46.0% yield).

HRMS (ESI⁺) C₃₈H₆₅FeIN₁₀NaO₁₁¹⁹⁵Pt[M+Na]²⁺ calc 619.1382, found 619.1328

HPLC (Grace Alltima C18 5 μm column, 25×4.6 mm) indicated that the product was 96.0% pure (retention time 14.0 min; gradient: 5 to 50% MeCN/0.1% TFA in water/0.1% TFA in 20 min measured at a wavelength of 430 nm).

### 6.13. Synthesis and Analytical Characterization of the Complex [AF-PEG₂-urea-pip-Pt(ethane-1,2-diamine)I]⁺ TFA⁻ (6m)

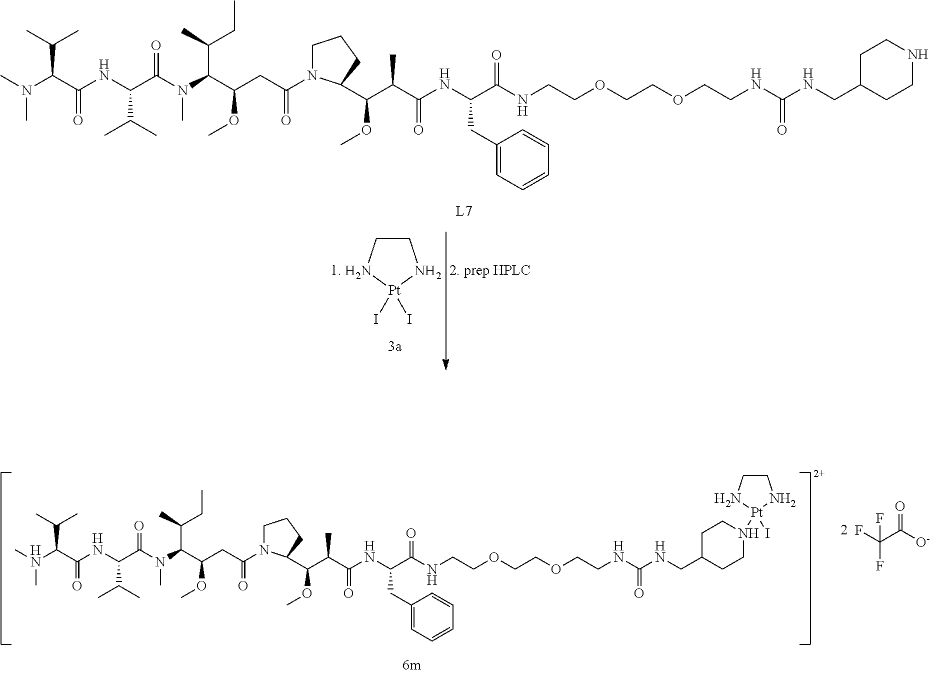

### 6.13.1. Synthesis of the Ligand AF-PEG2-urea-pip (L7)

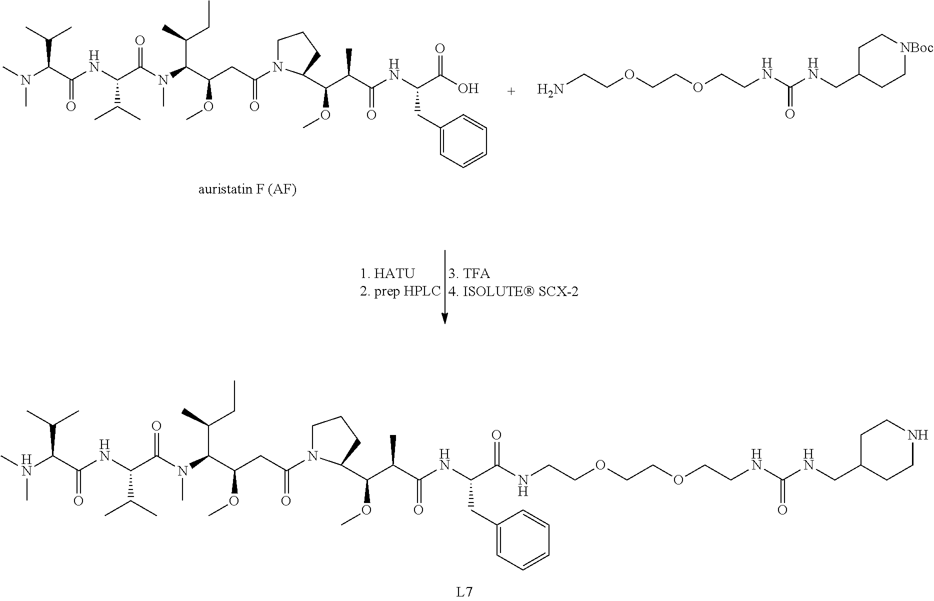

Auristatin F (AF) (40.0 mg, 54 μmol, 1.0 eq.), dissolved in DMF (1.33 mL), was added to tert-butyl 4-(12-amino-3-oxo-7,10-dioxa-2,4-diazadodecyl)piperidine-1-carboxylate (62.5 mg, 161 μmol, 3.0 eq.; synthesis is described in Sijbrandi et al., Cancer Res. 2017, 72, 257-267) in DMF (1 mL). HATU (40.8 mg, 107 μmol, 2.0 eq.) and DIPEA (29 μL, 161 μmol, 3.0 eq.) were subsequently added and the mixture was stirred for 1.5 h in an ice bath. The reaction mixture was concentrated, dissolved in water/MeCN (3.5:1, 3 mL), and filtered through a 0.2 μm syringe filter. Purification was performed by preparative reverse-phase HPLC (Grace Alltima C18 5 μm column, 22×250 mm; gradient: 30 to 50% MeCN/0.1% TFA in water/0.1% TFA in 36 min). Product fractions were concentrated under reduced pressure resulting in a colorless solid (56 mg, 85% yield).

HPLC (Grace Alltima C18 5 μm column, 25×4.6 mm) indicated that the product compound L₇-Boc was 100% pure (retention time 19.8 min; gradient: 5 to 50% MeCN/0.1% TFA in water/0.1% TFA in 20 min measured at a wavelength of 210 nm).

HRMS (ESI+) C₅₈H₁₀₂N₉O₁₂ [M+H]⁺ calc 1116.7642, found 1116.7774

The obtained compound L7-Boc was dissolved in DCM (2 mL) and TFA (2 mL) was added. The mixture was stirred for 45 min at room temperature, followed by concentration under reduced pressure. The residue was dissolved in 10% MeOH/DCM (2 mL) and loaded on an ISOLUTE® SCX-2 column, pre-washed with DCM (10 mL). The column was washed with 10% MeOH/DCM (20 mL), and the product was eluted with 1 M methanolic ammonia in DCM (1:1). The combined product fractions were concentrated under reduced pressure and co-evaporated with MeOH several times to remove traces of ammonia affording a colorless solid (34 mg, 73% yield).

HPLC (Grace Alltima C18 5 μm column, 25×4.6 mm) indicated that the product was 99% pure (retention time 9.2 min; gradient: 20 to 100% MeCN/0.1% TFA in water/0.1% TFA in 20 min measured at a wavelength of 210 nm).

HRMS (ESI+) C₅₃H₉₄N₉O₁₀ [M+H]⁺ calc 1016.7118, found 1016.6976

### 6.13.2. Synthesis of the Complex [AF-PEG₂-urea-pip-Pt(ethane-1,2-diamine)I]⁺ TFA⁻ (6m)

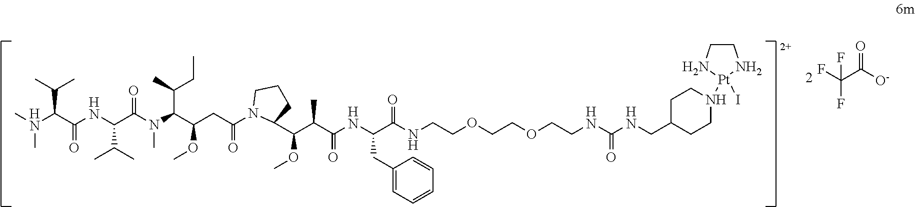

N-(3-Oxo-1-(piperidin-4-yl)-7,10-dioxa-2,4-diazadodecan-12-yl) AF amide (L7) (AF-pip; 15.0 mg, 15 μmol, 1.0 eq.) and Pt(ethane-1,2-diamine)I₂ (3a) (22.5 mg, 44 μmol, 3.0 eq.) were dissolved in dry DMF (150 μL) under argon atmosphere. Diisopropyamine (7.71 μL, 44 μmol, 3.0 eq.) was added and the course of the reaction was followed by HPLC. The reaction mixture was stirred at 60° C. for 2 h. At this moment, the reaction mixture contained 100.0% product.

The reaction mixture was diluted with water/MeOH (2:1, 2.5 mL) and filtered through a 0.2 μm syringe filter. Purification was performed by preparative reverse-phase HPLC (Grace Alltima C18 5 μm column, 22×250 mm; gradient: 35 to 100% MeOH/0.1% TFA in water/0.1% TFA in 36 min). Product fractions were concentrated under reduced pressure resulting in a colorless oil (18.0 mg, 75.0% yield).

HRMS (ESI⁺) C₅₅H₁₀₂IN₁₁O₁₀¹⁹⁵Pt[M+H]²⁺ calc 699.3247, found 699.3198

HPLC (Grace Alltima C18 5 μm column, 25×4.6 mm) indicated that the product was 98.9% pure (retention time 10.3 min; gradient: 20 to 100% MeCN/0.1% TFA in water/0.1% TFA in 20 min measured at a wavelength of 210 nm).

¹⁹⁵Pt-NMR (86 MHz, DMF-d₇): δ −3016

### 6.14. Synthesis of the Complex [AF-PEG₂-urea-pip-Pt((1R,2R)-cyclohexane-1,2-diamine)I]⁺ TFA⁻ (6n)

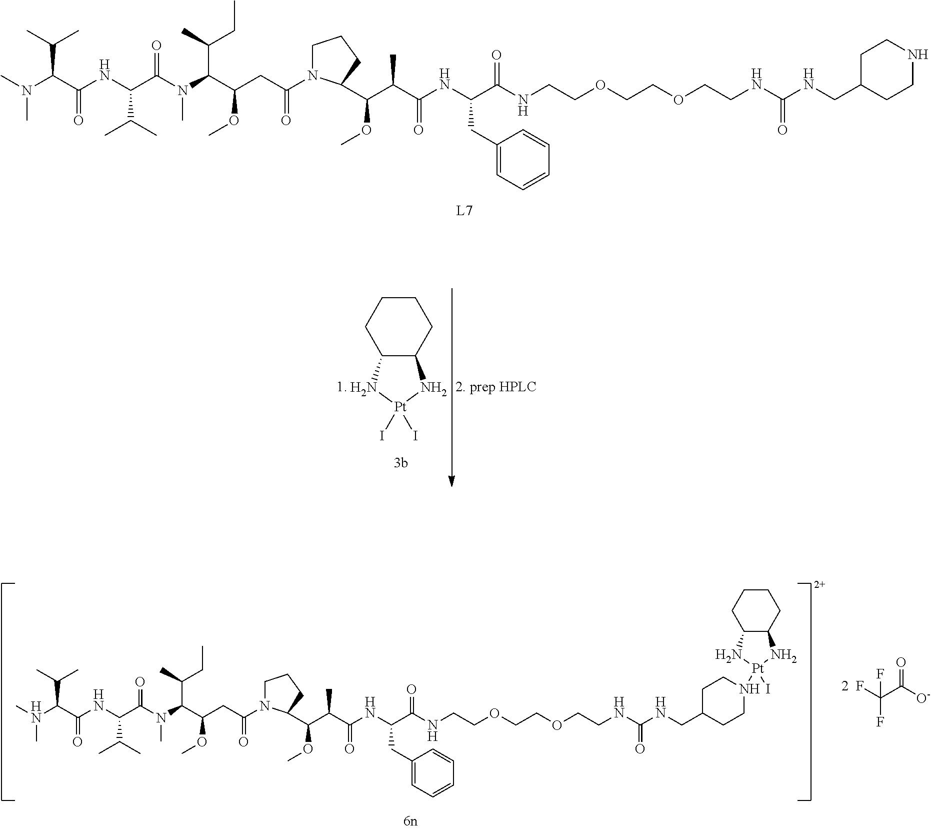

N-(3-Oxo-1-(piperidin-4-yl)-7,10-dioxa-2,4-diazadodecan-12-yl) AF amide (L7) (AF-pip; 15.0 mg, 15 μmol, 1.0 eq.) and Pt(((1R,2R)-(−)-1,2-diaminocyclohexane))I₂ (3b) (24.8 mg, 44 μmol, 3.0 eq.) were dissolved in dry DMF (150 μL) under argon atmosphere. Diisopropylethylamine (7.71 μL, 44 μmol, 3.0 eq.) was added and the course of the reaction was followed by HPLC. The reaction mixture was stirred at 60° C. for 4 h. At this moment, the reaction mixture contained 100.0% product.

The reaction mixture was diluted with water/MeOH (2:1, 2.5 mL) and filtered through a 0.2 μm syringe filter. Purification was performed by preparative reverse-phase HPLC (Grace Alltima C18 5 μm column, 22×250 mm; gradient: 35 to 100% MeOH/0.1% TFA in water/0.1% TFA in 36 min). Product fractions were concentrated under reduced pressure resulting in a colorless oil (15.6 mg, 59.0% yield).

HRMS (ESI⁺) C₅₉H₁₀₈IN₁₁O₁₀¹⁹⁵Pt[M+H]²⁺ calc 726.3481, found 726.3441

HPLC (Grace Alltima C18 5 μm column, 25×4.6 mm) indicated that the product was 99.4% pure (retention time 11.0 min; gradient: 20 to 100% MeCN/0.1% TFA in water/0.1% TFA in 20 min measured at a wavelength of 210 nm).

### 6.15. Synthesis of the Complex [AF-PEG₂-urea-pip-Pt((1S,2S)-cyclohexane-1,2-diamine)I]⁺ TFA⁻ (6o)

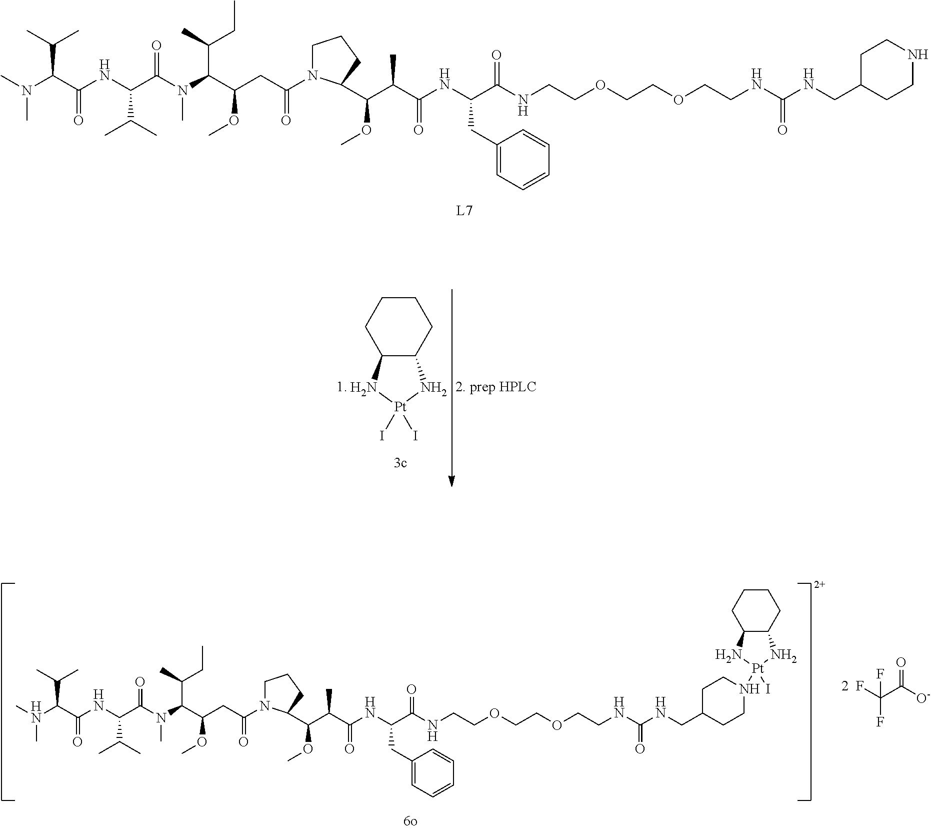

N-(3-Oxo-1-(piperidin-4-yl)-7,10-dioxa-2,4-diazadodecan-12-yl) AF amide (L7) (AF-pip; 16.0 mg, 16 μmol, 1.0 eq.) and Pt(((1S,2S)-(−)-1,2-diaminocyclohexane))I₂ (3c) (26.1 mg, 47 μmol, 3.0 eq.) were dissolved in dry DMF (150 μL) under argon atmosphere. Diisopropylethylamine (8.23 μL, 47 μmol, 3.0 eq.) was added and the course of the reaction was followed by HPLC. The reaction mixture was stirred at 60° C. for 18 h. At this moment, the reaction mixture contained 100.0% product. The reaction mixture was diluted with a water/MeOH solution (2:1, 2.5 mL) and filtered through a 0.2 μm syringe filter. Purification was performed by preparative reverse-phase HPLC (Grace Alltima C18 5 μm column, 22×250 mm; gradient: 35 to 100% MeOH/0.1% TFA in water/0.1% TFA in 36 min). Product fractions were concentrated under reduced pressure resulting in a colorless oil (18.4 mg, 71.1% yield).

HRMS (ESI+) C₅₉H₁₀₈IN₁₁O₁₀¹⁹⁵Pt[M+H]²⁺ calc 726.3481, found 726.3483

HPLC (Grace Alltima C18 5 μm column, 25×4.6 mm) indicated that the product was 96.6% pure (retention time 11.3 min; gradient: 20 to 100% MeCN/0.1% TFA in water/0.1% TFA in 20 min measured at a wavelength of 210 nm).

### 6.16. Synthesis of the Complex [AF-PEG₂-urea-pip-Pt((1R,2S)-cyclohexane-1,2-diamine)I]⁺ TFA⁻ (6p)

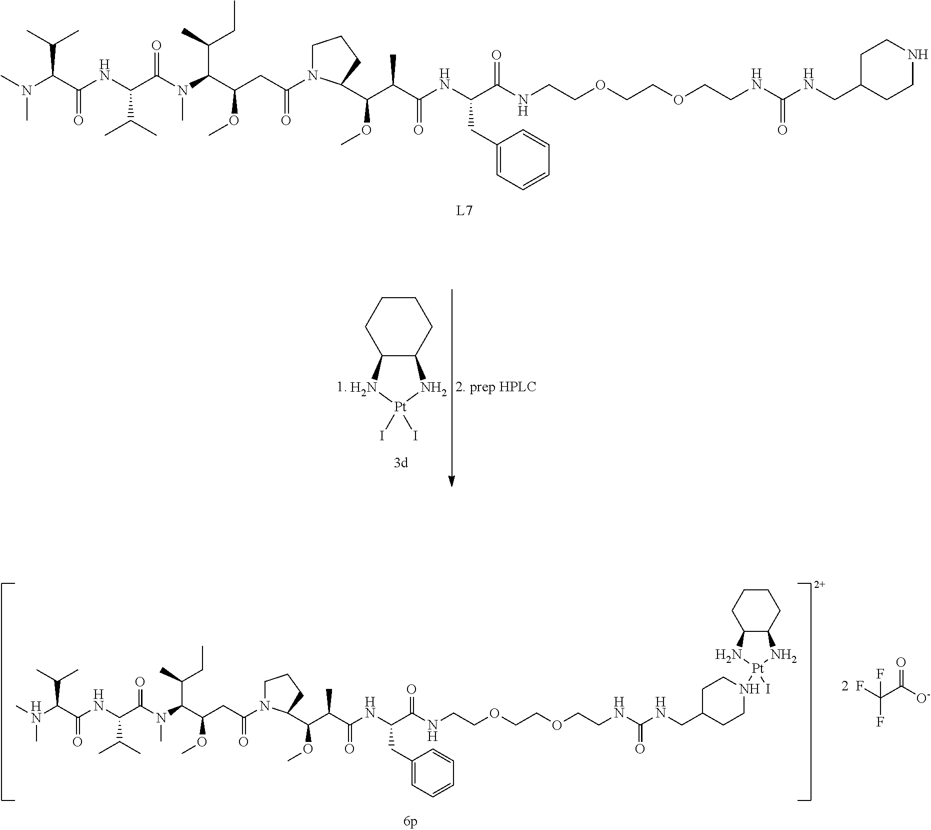

N-(3-Oxo-1-(piperidin-4-yl)-7,10-dioxa-2,4-diazadodecan-12-yl) AF amide (L7) (AF-pip; 20.0 mg, 20 μmol, 1.0 eq.) and Pt((1R,2S)-cyclohexane-1,2-diamine)I₂ (3d) (33.2 mg, 59 μmol, 3.0 eq.) were dissolved in dry DMF (150 μL) under argon atmosphere. Diisopropylethylamine (10.28 μL, 59 μmol, 3.0 eq.) was added and the course of the reaction was followed by HPLC. The reaction mixture was stirred at 60° C. for 18 h and subsequently the reaction mixture was diluted with water/MeOH (2:1, 2.5 mL) and filtered through a 0.2 μm syringe filter. Purification was performed by preparative reverse-phase HPLC (Grace Alltima C18 5 μm column, 22×250 mm; gradient: 35 to 100% B in 40 min, A: 95/5 Water/MeOH+0.1% TFA and B: 5/95 Water/MeOH+0.1% TFA). Product fractions were concentrated under reduced pressure resulting in a colorless oil (22.1 mg, 66.9% yield).

HRMS (ESI+) C₅₉H₁₀₇IN₁₁O₁₀¹⁹⁵Pt[M+H]⁺ calc 1451.6893, found 1451.6847

HPLC (Grace Alltima C18 5 μm column, 25×4.6 mm) indicated that the product was 98.6% pure (retention time 11.4 min; gradient: 20 to 100% MeCN/0.1% TFA in water/0.1% TFA in 20 min measured at a wavelength of 210 nm).

### 6.17. Synthesis of the Complex [AF-PEG₂-urea-pip-Pt(N¹,N²-dimethylethane-1,2-diamine)I]⁺ TFA⁻ (6q)

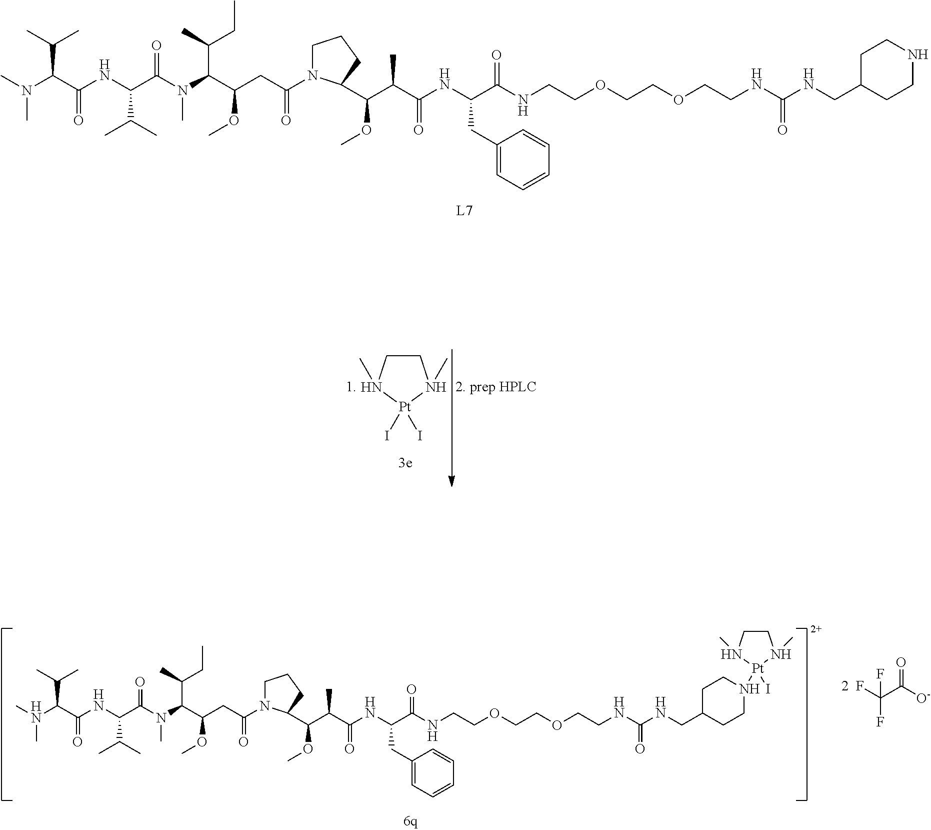

N-(3-Oxo-1-(piperidin-4-yl)-7,10-dioxa-2,4-diazadodecan-12-yl) AF amide (L7) (AF-pip; 20.0 mg, 20 μmol, 1.0 eq.) and Pt(N¹,N²-dimethylethane-1,2-diamine)I₂ (3e) (31.7 mg, 59 μmol, 3.0 eq.) were dissolved in dry DMF (150 μL) under argon atmosphere. Diisopropylethylamine (10.28 μL, 59 μmol, 3.0 eq.) was added and the course of the reaction was followed by HPLC. The reaction mixture was stirred at 60° C. for 18 h and subsequently the reaction mixture was diluted with water/MeOH (2:1, 2.5 mL) and filtered through a 0.2 μm syringe filter. Purification was performed by preparative reverse-phase HPLC (Grace Alltima C18 5 μm column, 22×250 mm; gradient: 35 to 100% B in 40 min, A: 95/5 Water/MeOH+0.1% TFA and B: 5/95 Water/MeOH+0.1% TFA). Product fractions were concentrated under reduced pressure resulting in a colorless oil (27.6 mg, 84.8% yield).

HRMS (ESI+) C₅₇H₁₀₅IN₁₁O₁₀¹⁹⁵Pt[M]⁺ calc 1425.6736, found 1425.6701

HPLC (Grace Alltima C18 5 μm column, 25×4.6 mm) indicated that the product was 97.0% pure (retention time 11.0 min; gradient: 20 to 100% MeCN/0.1% TFA in water/0.1% TFA in 20 min measured at a wavelength of 210 nm).

### 6.18. Synthesis of the Complex [AF-PEG₂-urea-pip-Pt(propane-1,3-diamine)I]⁺ TFA⁻ (6r)

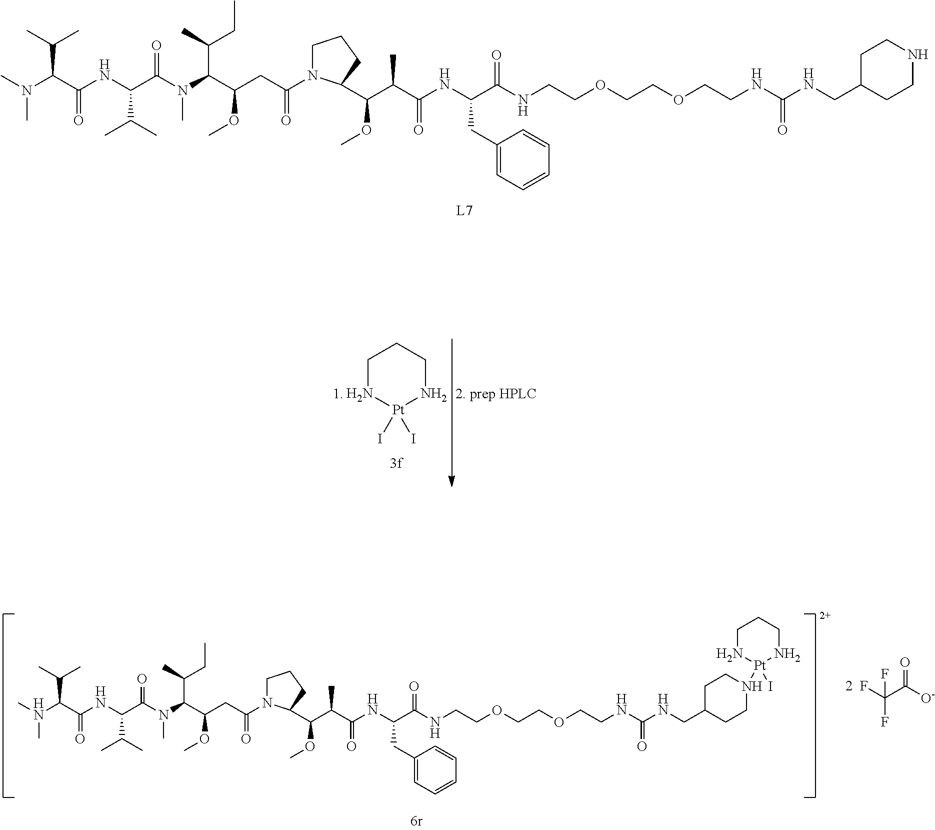

N-(3-Oxo-1-(piperidin-4-yl)-7,10-dioxa-2,4-diazadodecan-12-yl) AF amide (L7) (AF-pip; 16.0 mg, 16 μmol, 1.0 eq.) and PtI₂(propane-1,3-diamine) (3f) (24.7 mg, 47 μmol, 3.0 eq.) were dissolved in dry DMF (150 μL) under argon atmosphere. Diisopropylethylamine (8.23 μL, 47 μmol, 3.0 eq.) was added and the course of the reaction was followed by HPLC. The reaction mixture was stirred at 60° C. for 18 h and subsequently the reaction mixture was diluted with water/MeOH (2:1, 2.5 mL) and filtered through a 0.2 μm syringe filter. Purification was performed by preparative reverse-phase HPLC (Grace Alltima C18 5 μm column, 22×250 mm; gradient: 35 to 100% MeOH/0.1% TFA in water/0.1% TFA in 36 min). Product fractions were concentrated under reduced pressure resulting in a colorless oil (15.4 mg, 59.6% yield).

HRMS (ESI+) C₅₆H₁₀₄IN₁₁O₁₀¹⁹⁵Pt[M+H]²⁺ calc 706.3325, found 706.3344

HPLC (Grace Alltima C18 5 μm column, 25×4.6 mm) indicated that the product was 92.0% pure (retention time 10.5 min; gradient: 20 to 100% MeCN/0.1% TFA in water/0.1% TFA in 20 min measured at a wavelength of 210 nm).

### 6.19. Synthesis of the Complex [AF-PEG₂-urea-pip-Pt(1,3-diaminopropan-2-ol)I]⁺ TFA⁻ (6s)

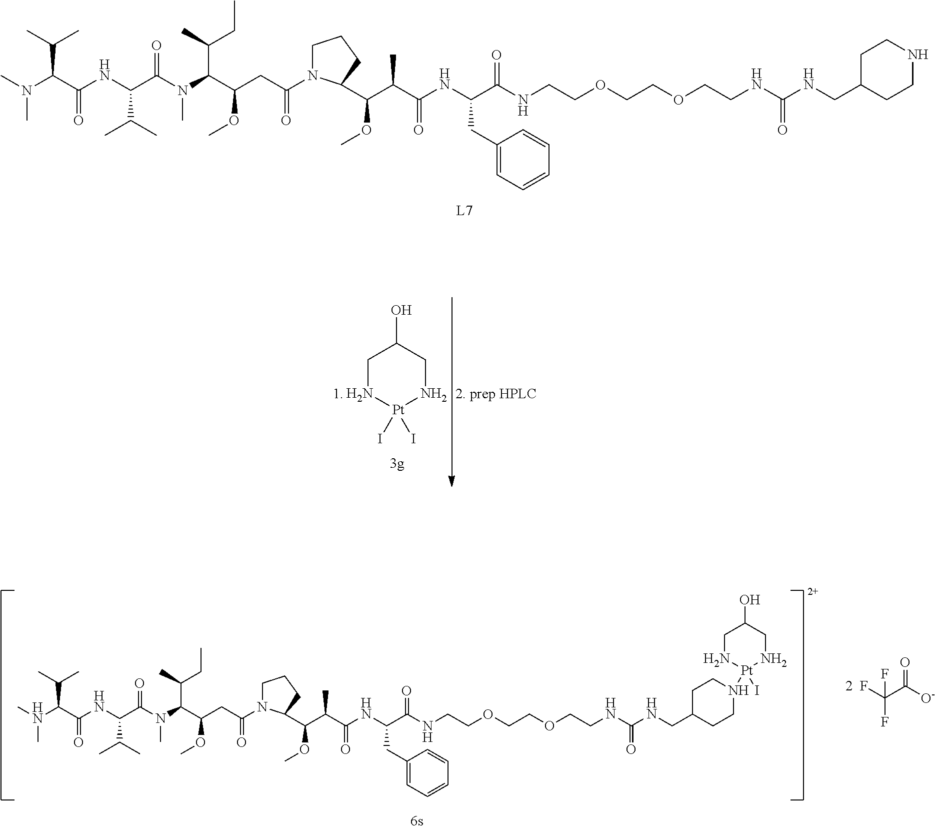

N-(3-Oxo-1-(piperidin-4-yl)-7,10-dioxa-2,4-diazadodecan-12-yl) AF amide (L7) (AF-pip; 15.0 mg, 15 μmol, 1.0 eq.) and Pt(1,3-diaminopropan-2-ol)I₂ (3g) (23.9 mg, 44 μmol, 3.0 eq.) were dissolved in dry DMF (150 μL) under argon atmosphere. Diisopropyethylamine (7.71 μL, 44 μmol, 3.0 eq.) was added and the course of the reaction was followed by HPLC. The reaction mixture was stirred at 60° C. for 2 h. At this moment, the reaction mixture contained 100.0% product.

The reaction mixture was diluted with water/MeOH (2:1, 2.5 mL) and filtered through a 0.2 μm syringe filter. Purification was performed by preparative reverse-phase HPLC (Grace Alltima C18 5 μm column, 22×250 mm; gradient: 35 to 100% MeOH/0.1% TFA in water/0.1% TFA in 36 min). Product fractions were concentrated under reduced pressure resulting in a colorless oil (14.5 mg, 59.4% yield).

HRMS (ESI⁺) C₅₆H₁₀₄IN₁₁O₁₁¹⁹⁵Pt[M+H]²⁺ calc 714.3299, found 714.3254

HPLC (Grace Alltima C18 5 μm column, 25×4.6 mm) indicated that the product was 94.4% pure (retention time 10.1 min; gradient: 20 to 100% MeCN/0.1% TFA in water/0.1% TFA in 20 min measured at a wavelength of 210 nm).

### 6.20. Synthesis of the Complex [AF-PEG₂-urea-pip-Pt(((1R,2R)-cyclobutane-1,2-diyl)dimethanamine)I]⁺ TFA⁻ (6t)

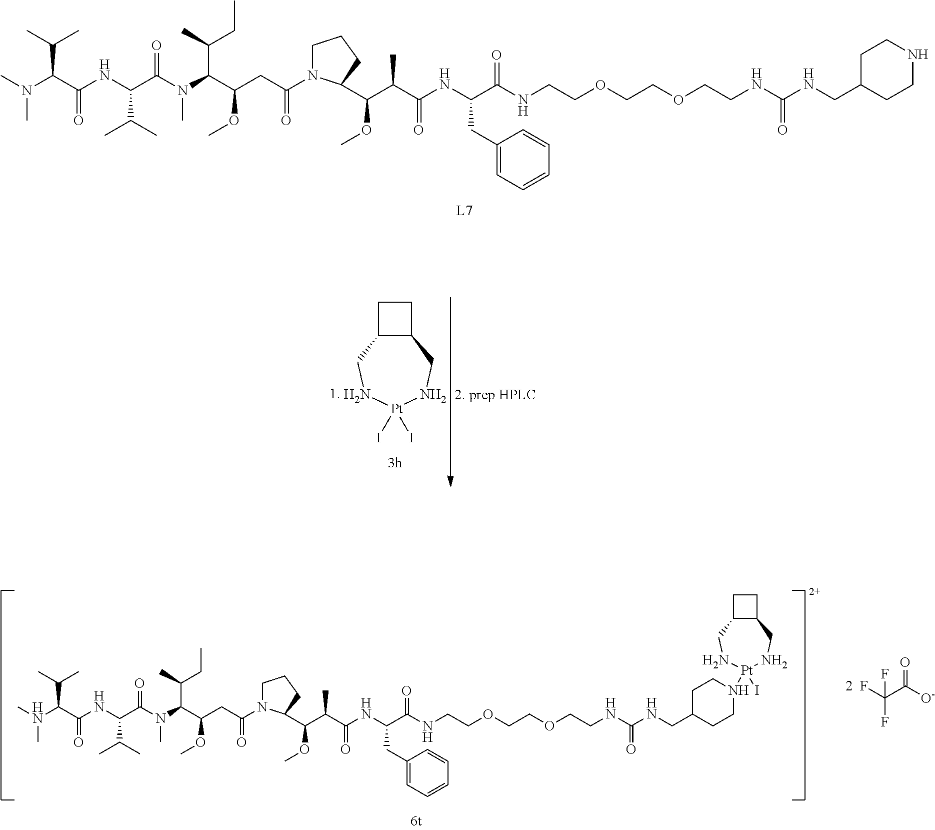

N-(3-Oxo-1-(piperidin-4-yl)-7,10-dioxa-2,4-diazadodecan-12-yl) AF amide (L7) (AF-pip; 15.0 mg, 15 μmol, 1.0 eq.) and Pt(((1R,2R)-cyclobutane-1,2-diyl)dimethanamine)I₂ (3h) (24.8 mg, 44 μmol, 3.0 eq.) were dissolved in dry DMF (150 μL) under argon atmosphere. Diisopropylethylamine (7.71 μL, 44 μmol, 3.0 eq.) was added and the course of the reaction was followed by HPLC. The reaction mixture was stirred at 60° C. for 2 h. At this moment, the reaction mixture contained 100.0% product.

The reaction mixture was diluted with water/MeOH (2:1, 2.5 mL) and filtered through a 0.2 μm syringe filter. Purification was performed by preparative reverse-phase HPLC (Grace Alltima C18 5 μm column, 22×250 mm; gradient: 35 to 100% MeOH/0.1% TFA in water/0.1% TFA in 36 min). Product fractions were concentrated under reduced pressure resulting in a colorless oil (8.6 mg, 34.7% yield).

HRMS (ESI⁺) C₅₉H₁₀₈IN₁₁O₁₀¹⁹⁵Pt[M+H]²⁺ calc 726.3481, found 726.3444

HPLC (Grace Alltima C18 5 μm column, 25×4.6 mm) indicated that the product was 98.7% pure (retention time 11.6 min; gradient: 20 to 100% MeCN/0.1% TFA in water/0.1% TFA in 20 min measured at a wavelength of 210 nm).

### 6.21. Synthesis of the Complex [AF-PEG₂-urea-pip-Pt((3R,4R,5S,6R)-3,4-diamino-6-(hydroxymethyl)tetrahydro-2H-pyran-2,5-diol)I]⁺ TFA⁻ (6u)

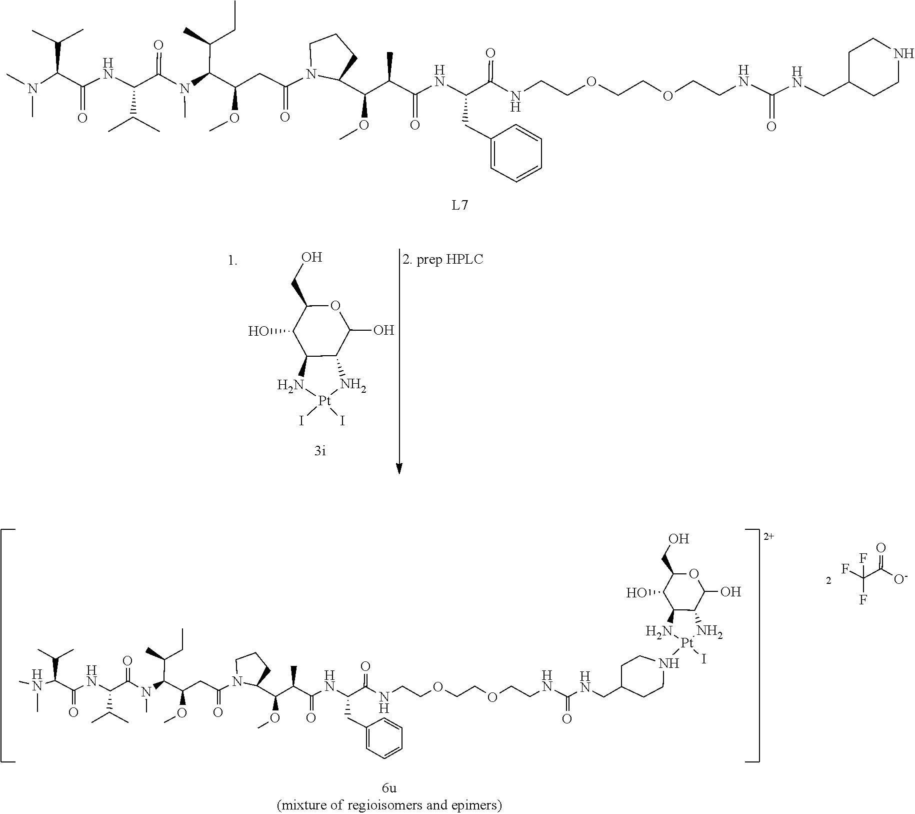

N-(3-Oxo-1-(piperidin-4-yl)-7,10-dioxa-2,4-diazadodecan-12-yl) AF amide (L7) (AF-pip; 15.0 mg, 15 μmol, 1.0 eq.) and Pt((3R,4R,5S,6R)-3,4-diamino-6-(hydroxymethyl)tetrahydro-2H-pyran-2,5-diol)I₂ (31) (27.8 mg, 44 μmol, 3.0 eq.) were dissolved in dry DMF (150 μL) under argon atmosphere. N,N-Diisopropylethylamine (7.71 μL, 44 μmol, 3.0 eq.) was added and the course of the reaction was followed by HPLC. The reaction mixture was stirred at 60° C. for 3.5 h. At this moment, the reaction mixture contained 63.7% product.

The reaction mixture was diluted with water/MeOH (2:1, 2.5 mL) and filtered through a 0.2 μm syringe filter. Purification was performed by preparative reverse-phase HPLC (Grace Alltima C18 5 μm column, 22×250 mm; gradient: 35 to 100% MeOH/0.1% TFA in water/0.1% TFA in 36 min). Product fractions were concentrated under reduced pressure resulting in a colorless oil (10.5 mg, 40.8% yield).

HRMS (ESI⁺) C₅₉H₁₀₈IN₁₁O₁₄¹⁹⁵Pt[M+H]⁺ calc 758.3379, found 758.3327

HPLC (Grace Alltima C18 5 μm column, 25×4.6 mm) indicated that the product was 98.9% pure (note: the product was obtained as a mixture of regioisomers and epimers, observed as a broad peak; retention time 9.5 min; gradient: 20 to 100% MeCN/0.1% TFA in water/0.1% TFA in 20 min measured at a wavelength of 210 nm).

### 6.22. Synthesis of the Complex [AF-PEG₂-urea-pip-Pt((4aR,6R,7R,8R,8aS)-6-methoxy-2-phenylhexahydropyrano[3,2-d][1,3]dioxine-7,8-diamine)I]⁺ TFA⁻ (6v)

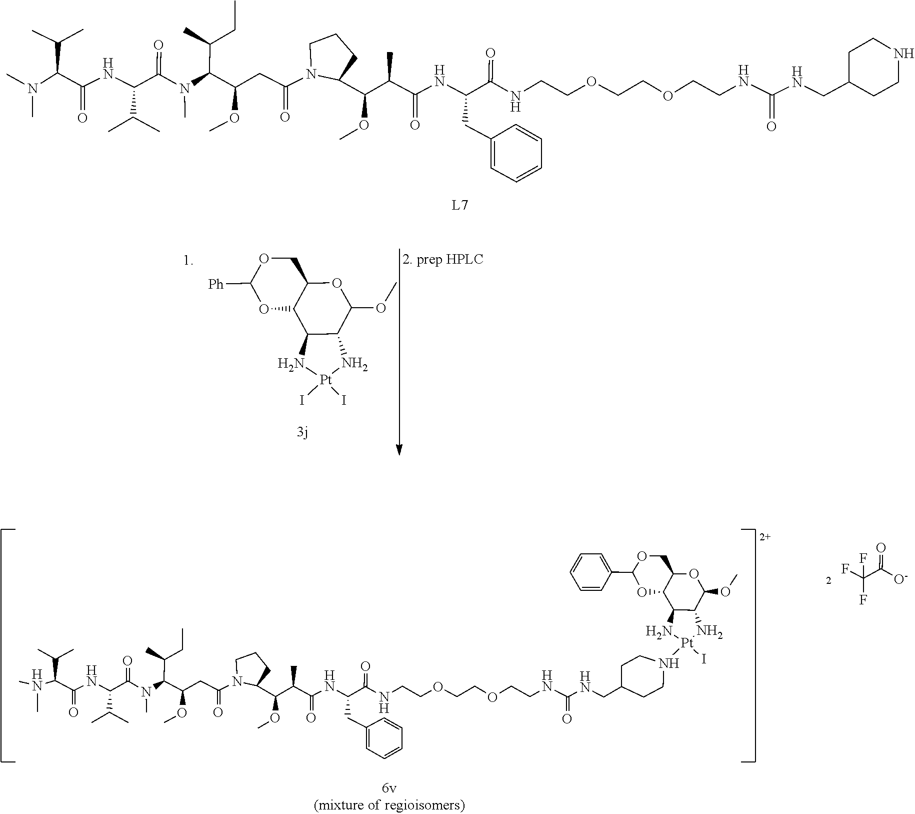

N-(3-Oxo-1-(piperidin-4-yl)-7,10-dioxa-2,4-diazadodecan-12-yl) AF amide (L7) (AF-pip; 16.0 mg, 16 μmol, 1.0 eq.) and Pt((4aR,6R,7R,8R,8aS)-6-methoxy-2-phenylhexahydropyrano[3,2-d][1,3]dioxine-7,8-diamine)I₂ (3j) (34.4 mg, 47 μmol, 3.0 eq.) were dissolved in dry DMF (150 μL) under argon atmosphere. Diisopropylethylamine (8.23 μL, 47 μmol, 3.0 eq.) was added and the course of the reaction was followed by HPLC. The reaction mixture was stirred at 60° C. for 18 h and subsequently the reaction mixture was diluted with water/MeOH (2:1, 2.5 mL) and filtered through a 0.2 μm syringe filter. Purification was performed by preparative reverse-phase HPLC (Grace Alltima C18 5 μm column, 22×250 mm; gradient: 35 to 100% MeOH/0.1% TFA in water/0.1% TFA in 36 min). Product fractions were concentrated under reduced pressure resulting in a colorless oil (21.6 mg, 74.3% yield).

HRMS (ESI+) C₆₇H₁₁₄IN₁₁O₁₄¹⁹⁵Pt[M+H]²⁺ calc 809.3614, found 809.3633

HPLC (Grace Alltima C18 5 μm column, 25×4.6 mm) indicated that the product was 96.3% pure (note: the product was obtained as a mixture of regioisomers, so that two peaks were observed; retention times 12.8 min and 13.2 min; gradient: 20 to 100% MeCN/0.1% TFA in water/0.1% TFA in 20 min measured at a wavelength of 210 nm).

### 6.23. Synthesis of the Complex [AF-PEG₂-urea-pip-Pt(2-((2-aminoethyl)amino)ethan-1-ol)I]⁺ TFA⁻ (6w)

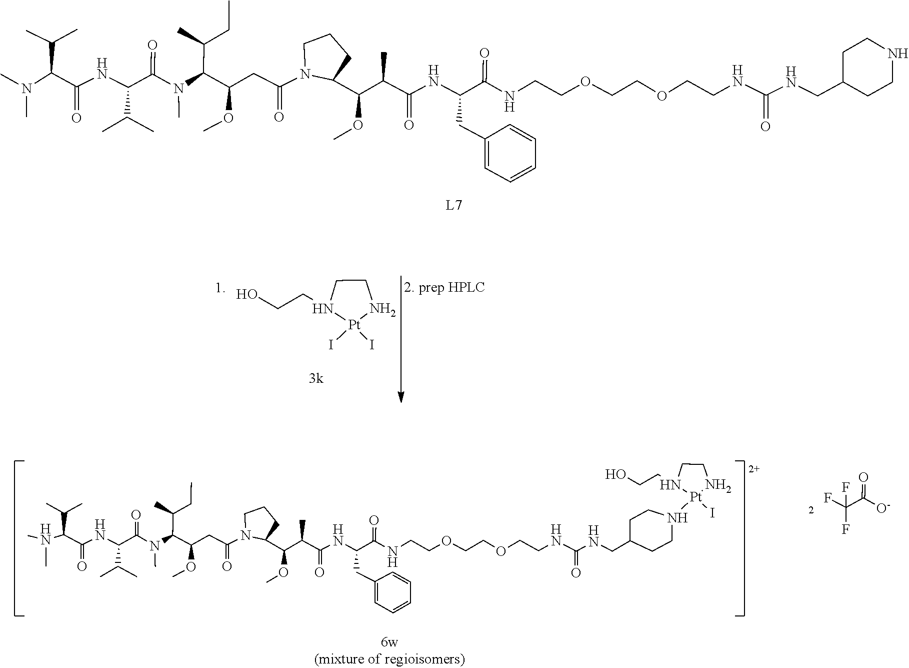

N-(3-Oxo-1-(piperidin-4-yl)-7,10-dioxa-2,4-diazadodecan-12-yl) AF amide (L7) (AF-pip; 16.0 mg, 16 μmol, 1.0 eq.) and Pt(2-((2-aminoethyl)amino)ethan-1-ol)I₂ (3k) (26.1 mg, 47 μmol, 3.0 eq.) were dissolved in dry DMF (150 μL) under argon atmosphere. Diisopropylethylamine (8.23 μL, 47 μmol, 3.0 eq.) was added and the course of the reaction was followed by HPLC. The reaction mixture was stirred at 60° C. for 18 h and subsequently the reaction mixture was diluted with water/MeOH (2:1, 2.5 mL) and filtered through a 0.2 μm syringe filter. Purification was performed by preparative reverse-phase HPLC (Grace Alltima C18 5 μm column, 22×250 mm; gradient: 35 to 100% MeOH/0.1% TFA in water/0.1% TFA in 36 min). Product fractions were concentrated under reduced pressure resulting in a colorless oil (17.4 mg, 66.2% yield).

HRMS (ESI+) C₅₇H₁₀₆IN₁₁O₁₁¹⁹⁵Pt[M+H]²⁺ calc 721.3377, found 721.3379

HPLC (Grace Alltima C18 5 μm column, 25×4.6 mm) indicated that the product was 98.8% pure (note: the product was obtained as a mixture of presumably (regio)isomers, so that three peaks were observed; retention times 9.0 min, 10.1 min, and 10.4 min; gradient: 20 to 100% MeCN/0.1% TFA in water/0.1% TFA in 20 min measured at a wavelength of 210 nm).

### 6.24. Synthesis of the Complex [AF-PEG₂-urea-pip-Pt(2,2′-(ethane-1,2-diylbis(azanediyl))bis(ethan-1-ol))I]⁺ TFA⁻ (6x)

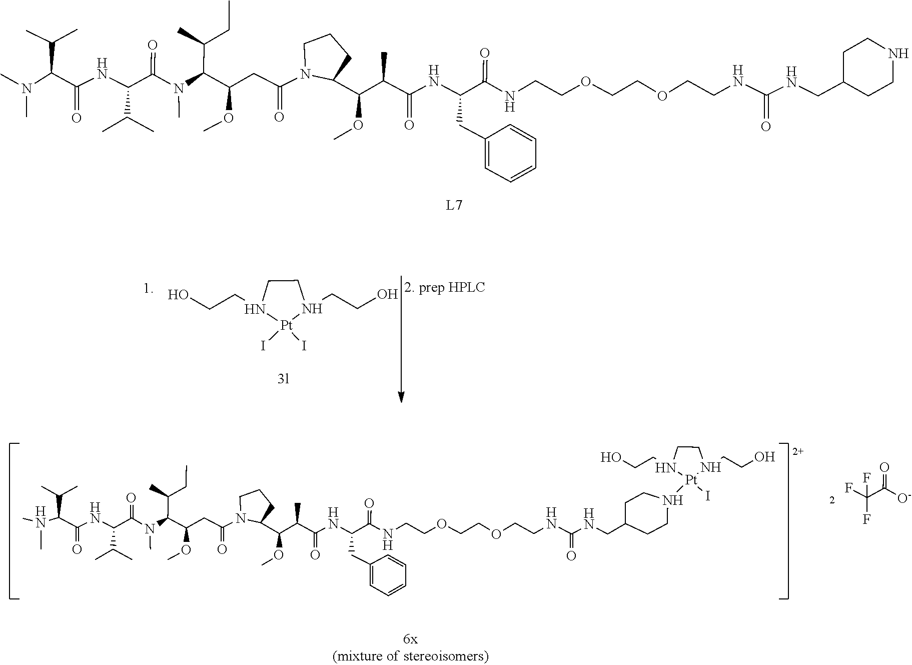

N-(3-Oxo-1-(piperidin-4-yl)-7,10-dioxa-2,4-diazadodecan-12-yl) AF amide (L7) (AF-pip; 16.0 mg, 16 μmol, 1.0 eq.) and Pt(2,2′-(ethane-1,2-diylbis(azanediyl))bis(ethan-1-ol))I₂ (31) (28.2 mg, 47 μmol, 3.0 eq.) were dissolved in dry DMF (150 μL) under argon atmosphere. Diisopropylethylamine (8.23 μL, 47 μmol, 3.0 eq.) was added and the course of the reaction was followed by HPLC. The reaction mixture was stirred at 60° C. for 18 h and subsequently the reaction mixture was diluted with water/MeOH (2:1, 2.5 mL) and filtered through a 0.2 μm syringe filter. Purification was performed by preparative reverse-phase HPLC (Grace Alltima C18 5 μm column, 22×250 mm; gradient: 35 to 100% MeOH/0.1% TFA in water/0.1% TFA in 36 min). Product fractions were concentrated under reduced pressure resulting in a colorless oil (10.5 mg, 38.9% yield).

HRMS (ESI+) C₅₉H₁₁₀IN₁₁O₁₂¹⁹⁵Pt[M+H]²⁺ calc 743.3508, found 743.3528

HPLC (Grace Alltima C18 5 μm column, 25×4.6 mm) indicated that the product was 93.7% pure (note: the product was obtained as a mixture of presumably stereoisomers, so that two peaks were observed; retention times 9.0 min and 10.2 min; gradient: 20 to 100% MeCN/0.1% TFA in water/0.1% TFA in 20 min measured at a wavelength of 210 nm).

### 6.25. Synthesis and Analytical Characterization of the Complex [AF-pip-Pt(ethane-1,2-diamine)I]⁺ TFA⁻ (6y)

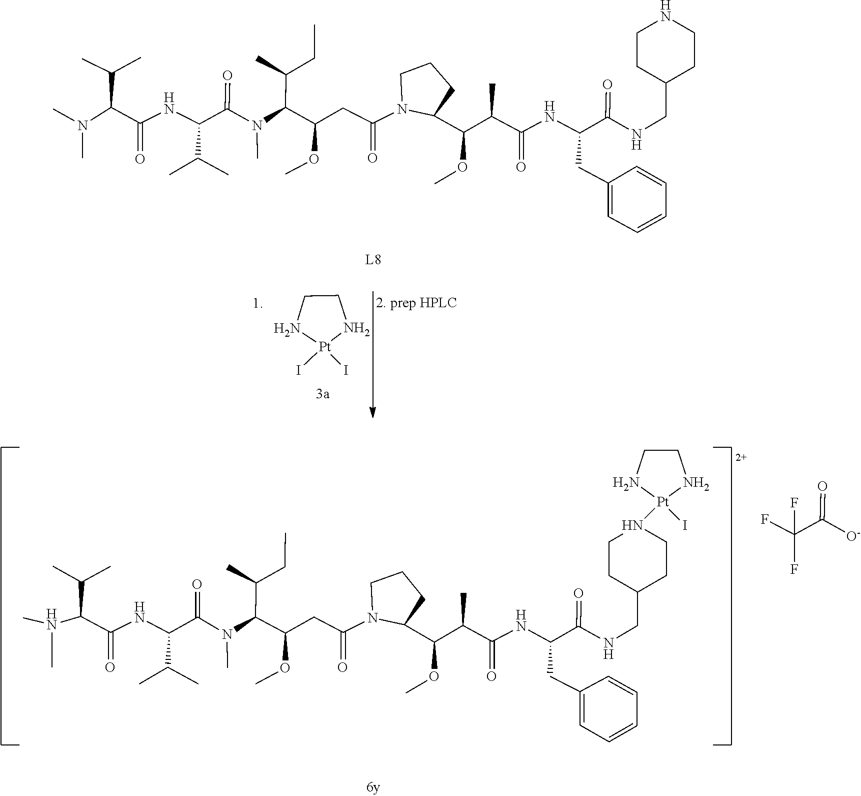

### 6.25.1. Synthesis of the Ligand AF-pip (L8)

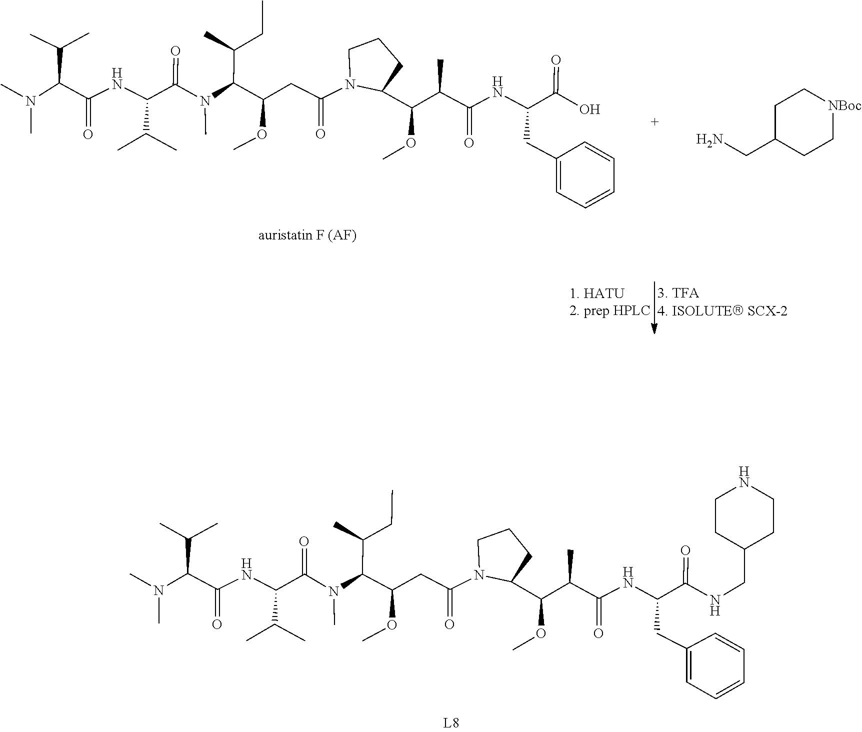 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C)C(C)C ; CC(C)(C)OC(=O)N1CCC(CN)CC1 ; CC[C@H](C)[C@@H]([C@@H](CC(=O)N1CCC[C@H]1[C@H](OC)[C@@H](C)C(=O)N[C@@H](Cc1ccccc1)C(=O)NCC1CCNCC1)OC)N(C)C(=O)[C@@H](NC(=O)[C@H](C(C)C)N(C)C)C(C)C]

Auristatin F (AF) (30.0 mg, 40 μmol, 1.0 eq.), dissolved in DMF (1.00 mL), was added to tert-butyl 4-(aminomethyl)piperidine-1-carboxylate (22.9 mg, 60 μmol, 1.5 eq). HATU (12.9 mg, 60 μmol, 1.5 eq.) and DIPEA (13.96 μL, 101 μmol, 2.5 eq.) were subsequently added and the mixture was stirred for 1 h in an ice bath. The reaction mixture was concentrated, dissolved in water/MeCN (3.5:1, 3 mL), and filtered through a 0.2 μm syringe filter. Purification was performed by preparative reverse-phase HPLC (Grace Alltima C18 5 μm column, 22×250 mm; gradient: 20 to 100% MeCN/0.1% TFA in water/0.1% TFA in 36 min). Product fractions were concentrated resulting in a colorless solid (44.5 mg, quant.).

HPLC (Grace Alltima C18 5 μm column, 25×4.6 mm) indicated that the product compound L8-Boc was 100.0% pure (95.9% compound L8-Boc: retention time 14.9 min and 4.1% Boc-deprotected compound L8: retention time 9.3 min; gradient: 20 to 100% MeCN/0.1% TFA in water/0.1% TFA in 20 min measured at a wavelength of 210 nm).

The obtained compound L8-Boc was dissolved in DCM (2 mL) and TFA (2 mL) was added. The mixture was stirred for 45 min at room temperature, followed by concentration under reduced pressure. The residue was dissolved in 10% MeOH/DCM (2 mL) and loaded on an ISOLUTE® SCX-2 column, pre-washed with DCM (10 mL). The column was washed with 10% MeOH/DCM (20 mL), and the product was eluted with 1 M methanolic ammonia in DCM (1:1). The combined product fractions were concentrated and co-evaporated with MeOH several times to remove traces of ammonia affording a colorless solid (22.7 mg, 63.0% yield).

HPLC (Grace Alltima C18 5 μm column, 25×4.6 mm) indicated that the product was 100% pure (retention time 9.3 min; gradient: 20 to 100% MeCN/0.1% TFA in water/0.1% TFA in 20 min measured at a wavelength of 210 nm).

HRMS (ESI+) C₄₆H₈₁N₇O₇ [M+2H]²⁺ calc 421.8093, found 421.8071

### 6.25.2. Synthesis of the Complex [AF-pip-Pt(ethane-1,2-diamine)I]⁺ TFA⁻ (6y)

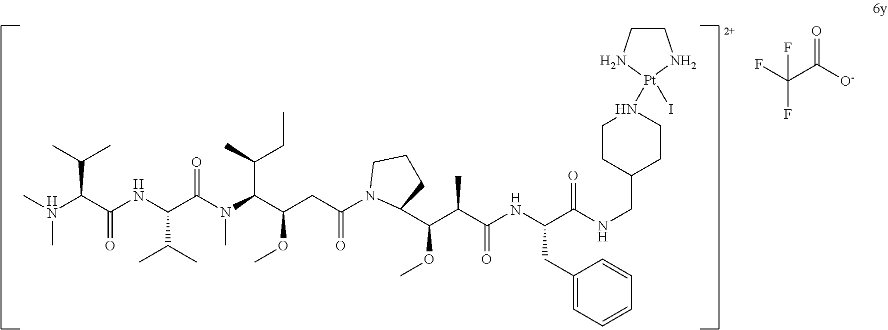

Auristatin F piperidinyl amide (L8) (AF-pip; 15.0 mg, 18 μmol, 1.0 eq.) and Pt(ethane-1,2-diamine)I₂ (3a) (27.2 mg, 53 μmol, 3.0 eq.) were dissolved in dry DMF (150 μL) under argon atmosphere. N,N-Diisopropylethylamine (9.33 μL, 53 μmol, 3.0 eq.) was added and the course of the reaction was followed by HPLC. The reaction mixture was stirred at 60° C. for 3.5 h. At this moment, the reaction mixture contained 100.0% product.

The reaction mixture was diluted with water/MeOH (2:1, 2.5 mL) and filtered through a 0.2 μm syringe filter. Purification was performed by preparative reverse-phase HPLC (Grace Alltima C18 5 μm column, 22×250 mm; gradient: 35 to 100% MeOH/0.1% TFA in water/0.1% TFA in 36 min). Product fractions were concentrated resulting in a colorless oil (15.3 mg, 59.1% yield).

HRMS (ESI⁺) C₄₈H₈₈IN₉O₇¹⁹⁵Pt[M+H]²⁺ calc 612.2744, found 612.2681

HPLC (Grace Alltima C18 5 μm column, 25×4.6 mm) indicated that the product was 97.5% pure (retention time 10.5 min; gradient: 20 to 100% MeCN/0.1% TFA in water/0.1% TFA in 20 min measured at a wavelength of 210 nm).

### 6.26. Synthesis and Analytical Characterization of the Complex [N-(14-azido-3,6,9,12-tetraoxatetradecyl)-3-(pyridin-4-yl)propanamide-Pt(ethane-1,2-diamine)I]⁺ TFA⁻ (6z)

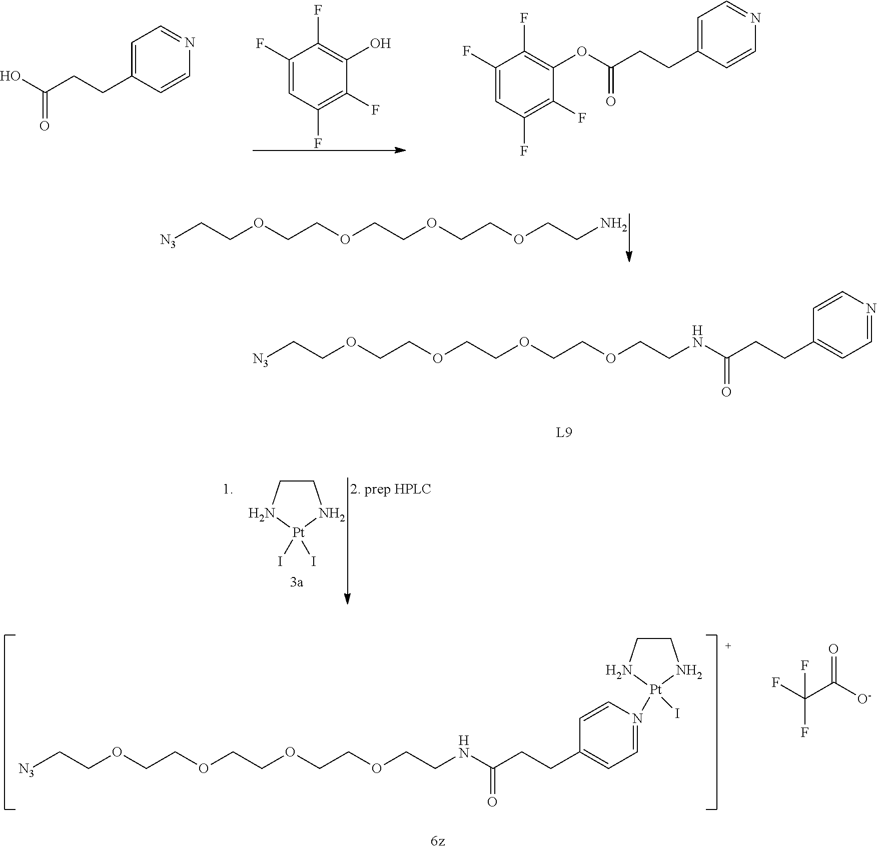

### 6.26.1. Synthesis of 2,3,5,6-tetrafluorophenyl 3-(pyridin-4-yl)propanoate

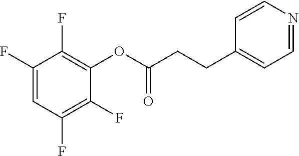 [SMILES: O=C(CCc1ccncc1)Oc1c(F)c(F)cc(F)c1F]

To a solution of 2,3,5,6-tetrafluorophenol (576 mg, 3.47 mmol, 1.1 eq.) in DCM (25 mL) was added 3-(pyridin-4-yl)propanoic acid (477 mg, 3.16 mmol, 1.0 eq.). The reaction mixture was stirred for 5 min at room temperature and EDC (726 mg, 3.79 mmol, 1.2 eq.) was added at room temperature. The resulting suspension was stirred for 60 h at room temperature. The reaction mixture was diluted with DCM (20 mL) and the mixture was washed with an aqueous 0.1 M HCl solution (prepared from 22.5 mL water and 2.5 mL 1 M HCl). The organic phase was subsequently washed with sat. NaHCO₃ solution and brine, dried with Na₂SO₄, and evaporated to dryness to obtain the crude product as a colorless solid (317 mg, 33.6% yield).

HPLC (Grace Alltima C18 5 μm column, 25×4.6 mm) indicated that the product was 96.5% pure (retention time 10.9 min; gradient: 20 to 100% MeCN/0.1% TFA in water/0.1% TFA in 20 min measured at a wavelength of 210 nm).

¹H NMR (400 MHz, DMSO-d₆): δ 8.54-8.39 (m, 2H), 8.00-7.80 (m, 1H), 7.39-7.25 (m, 2H), 3.24-3.15 (m, 2H), 3.06-2.94 (m, 2H).

### 6.26.2. Synthesis of the Ligand N-(14-azido-3,6,9,12-tetraoxatetradecyl)-3-(pyridin-4-yl)propanamide (N₃-PEG₄-py, L9)

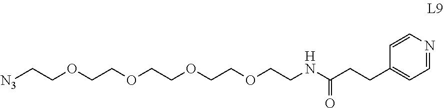 [SMILES: [N-]=[N+]=NCCOCCOCCOCCOCCNC(=O)CCc1ccncc1]

14-Azido-3,6,9,12-tetraoxatetradecan-1-amine (47.3 μL, 201 μmol, 1.0 eq.) and 2,3,5,6-tetrafluorophenyl 3-(pyridin-4-yl)propanoate (60 mg, 201 μmol, 1.0 eq.) were dissolved in dry MeCN (2 mL) under argon atmosphere. This mixture was stirred for 2.5 h (the reaction progress was monitored by TLC using cyclohexane/EtOAc 1:2 and ⁱPrOH/NH_{3 (aq.)}=10:1 as eluents). Then, TEA (27.9 μL, 201 μmol, 1.0 eq.) was added and the mixture was stirred for 20 h. After that, solvents were removed under reduced pressure to afford a colorless oily residue (119 mg) which was subsequently purified by column chromatography (step wise gradient using DCM/MeOH/NH_{3 (aq.)}=100:5:1→100:7.5:1→100:10:1 as an eluent). The product containing fraction was evaporated under reduced pressure to afford a colorless oil (66 mg, 83% yield).

HRMS (ESI+) C₁₈H₃₀N₅O₅ [M+H]⁺ calc 396.2241, found 396.2260

### 6.26.3. Synthesis of the Complex [N-(14-azido-3,6,9,12-tetraoxatetradecyl)-3-(pyridin-4-yl)propanamide-Pt(ethane-1,2-diamine)I]⁺ TFA⁻ (6z)

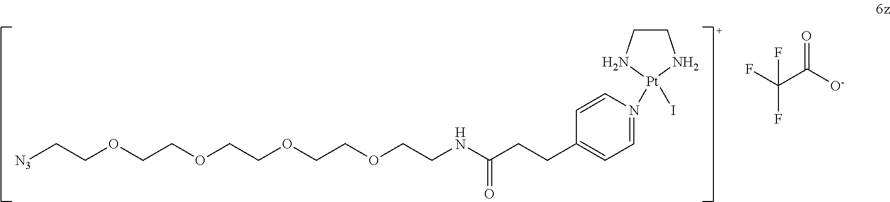

N-(14-Azido-3,6,9,12-tetraoxatetradecyl)-3-(pyridin-4-yl)propanamide (L9) (N₃-PEG₄-py; 22.5 mg, 57 μmol, 1.0 eq.) and Pt(ethane-1,2-diamine)I₂ (3a) (87.0 mg, 171 μmol, 3.0 eq.) were dissolved in dry DMF (500 μL) under argon atmosphere. Diisopropylethylamine (29.7 μL, 171 μmol, 3.0 eq.) was added, the reaction mixture was stirred at 40° C. for 24 h, and the course of the reaction was followed by HPLC. The reaction mixture was diluted with a 10 mM NaI/MeOH mixture (4:1, 2.5 mL) and filtered through a 0.2 μm syringe filter. Purification was performed by preparative reverse-phase HPLC (Grace Alltima C18 5 μm column, 22×250 mm; gradient: 20 to 75% MeOH/0.1% TFA in water/0.1% TFA in 36 min). Product fractions were concentrated under reduced pressure affording a colorless oil (36.8 mg, 72.6% yield).

HRMS (ESI+) C₂₀H₃₇IN₇O₅¹⁹⁵Pt[M]⁺ calc 777.1543, found 777.1540

HPLC (Grace Alltima C18 5 μm column, 25×4.6 mm) indicated that the product was 95.7% pure (retention time 16.6 min; gradient: 5 to 50% MeCN/0.1% TFA in water/0.1% TFA in 20 min measured at a wavelength of 210 nm).

### 6.27. Synthesis and Analytical Characterization of the Complex [N¹,N³-bis(14-azido-3,6,9,12-tetraoxatetradecyl)-N⁵-(pyridin-4-ylmethyl)benzene-1,3,5-tricarboxamide-Pt(ethane-1,2-diamine)I]⁺ TFA⁻ (6aa)

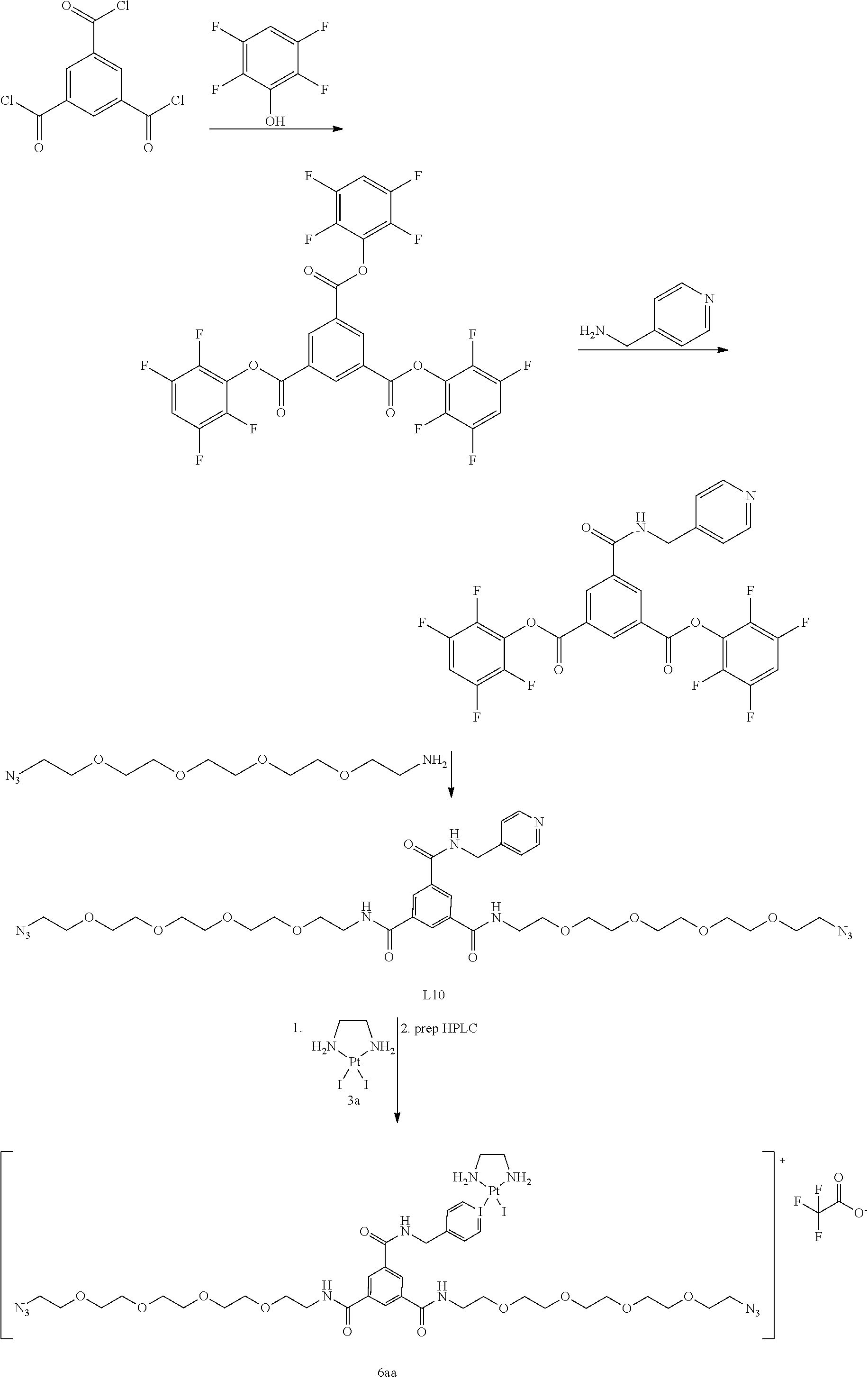

### 6.27.1. Synthesis of tris(2,3,5,6-tetrafluorophenyl) benzene-1,3,5-tricarboxylate

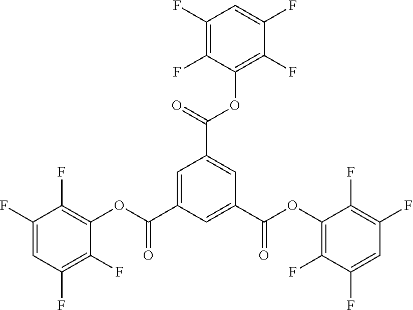 [SMILES: O=C(Oc1c(F)c(F)cc(F)c1F)c1cc(C(=O)Oc2c(F)c(F)cc(F)c2F)cc(C(=O)Oc2c(F)c(F)cc(F)c2F)c1]

Under argon atmosphere, DIPEA (13.9 mL, 80 mmol, 4.0 eq.) and 2,3,5,6-tetrafluorophenol (10.3 g, 60.4 mmol, 3.0 eq.) were dissolved in dry DCM (100 mL) and were subsequently added dropwise over 2.5 h to a rigorously stirred solution of benzene-1,3,5-tricarbonyl trichloride (3.57 mL, 20.0 mmol, 1.0 eq.) in dry DCM (150 mL) at 0° C. After addition, the mixture was stirred for 40 min and was allowed to warm to 6° C., after which it was gradually heated to the ambient temperature and stirred for another 1 h. Then, the reaction mixture was washed with 1 M HCl (320 mL) and with 1 M NaOH (320 mL). The alkaline aqueous layer was extracted with DCM (50 mL) and the combined organic layers were washed with brine (100 mL). The organic phase was dried with Na₂SO₄, filtered, and evaporated under reduced pressure. After removal of solvents, a pale brown solid (12.1 g, 93% yield) was obtained.

¹H NMR (400 MHz, CDCl₃): δ 9.30 (s, 3H), 7.17-7.05 (m, 3H)

### 6.27.2. Synthesis of bis(2,3,5,6-tetrafluorophenyl) 5-((pyridin-4-ylmethyl)carbamoyl)isophthalate

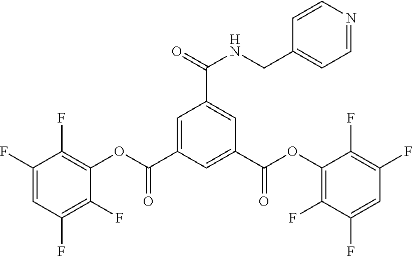 [SMILES: O=C(NCc1ccncc1)c1cc(C(=O)Oc2c(F)c(F)cc(F)c2F)cc(C(=O)Oc2c(F)c(F)cc(F)c2F)c1]

Tris(2,3,5,6-tetrafluorophenyl) benzene-1,3,5-tricarboxylate (5.00 g, 7.64 mmol, 3.0 eq.) was dissolved in DCM (100 mL). To this solution the mixture of pyridin-4-ylmethanamine (259 μL, 2.55 mmol, 1.0 eq.) and TEA (710 μL, 5.09 mmol, 2.0 eq.) in DCM (50 mL) was added dropwise over 140 min under vigorous stirring. Then, the mixture was stirred for another 1.5 h, after which TLC (DCM/MeOH/NH_{3 (aq.)}=100:10:1 as an eluent) indicated a full consumption of pyridin-4-ylmethanamine. The solvents were removed under reduced pressure and the residue was suspended in cyclohexane/EtOAc (3:12, 15 mL), sonicated in ultrasound bath, filtered, and the filter cake was washed with cyclohexane/EtOAc (1:2, 4 mL). TLC revealed that the filter cake contained tris(2,3,5,6-tetrafluorophenyl) benzene-1,3,5-tricarboxylate starting material and the filtrate contained product along with this starting material. Therefore, the filtrate was evaporated under reduced pressure and the crude residue was suspended in cyclohexane/EtOAc (1.5:6, 7.5 mL), sonicated in ultrasound bath, filtered, and the filter cake was washed with cyclohexane/EtOAc (1:4, 3 mL). The yield of the recovered tris(2,3,5,6-tetrafluorophenyl) benzene-1,3,5-tricarboxylate was 1.67 g (33.3% of the applied amount). Finally, the filtrate was evaporated under reduced pressure, the residue was dissolved in cyclohexane/EtOAc (1:1, 12 mL) and purified by column chromatography (step wise gradient using cyclohexane/EtOAc 2:1→1:1 as an eluent). The collected product containing fractions were evaporated under reduced pressure, the residue was dissolved in DCM (100 mL). The obtained organic phase was washed with 1 M NaOH (40 mL), dried with Na₂SO₄, filtered, and evaporated affording a colorless solid (691 mg, 46% yield).

¹H NMR (400 MHz, DMSO-d₆): δ 9.77-9.70 (m, 1H), 9.09-9.05 (m, 2H), 8.97 (s, 1H), 8.54-8.50 (m, 2H), 8.12-7.97 (m, 2H), 7.38-7.34 (m, 2H), 4.58 (d, J=5.7 Hz, 2H).

### 6.27.3. Synthesis of the Ligand N¹,N³-bis(14-azido-3,6,9,12-tetraoxatetradecyl)-N⁵-(pyridin-4-ylmethyl)benzene-1,3,5-tricarboxamide (bis-N₃-PEG₄-benzene-py, L10)

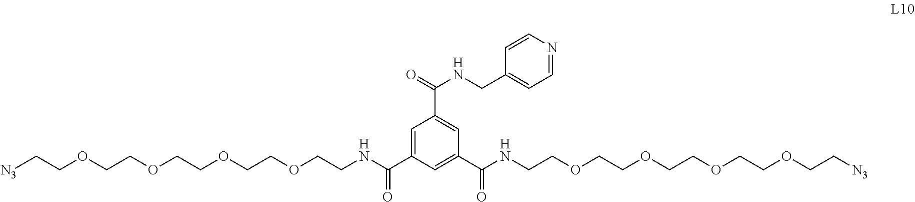 [SMILES: [N-]=[N+]=NCCOCCOCCOCCOCCNC(=O)c1cc(C(=O)NCCOCCOCCOCCOCCN=[N+]=[N-])cc(C(=O)NCc2ccncc2)c1]

Bis(2,3,5,6-tetrafluorophenyl) 5-((pyridin-4-ylmethyl)carbamoyl)isophthalate (88 mg, 0.15 mmol, 1.0 eq.) was dissolved in dry EtOAc/THF (5:2, 7 mL), followed by the addition of 14-azido-3,6,9,12-tetraoxatetradecan-1-amine (79 mg, 0.3 mmol, 2.0 eq.; dissolved in EtOAc (0.5 mL)), and TEA (61.7 μL, 0.44 mmol, 3.0 eq.). The resulting mixture was stirred under argon atmosphere at room temperature for 20 h. TLC (cyclohexane/EtOAc=1:2 and DCM/MeOH/NH_{3 (aq.)}=100:10:1 as eluents) showed a full consumption of both bis(2,3,5,6-tetrafluorophenyl) 5-((pyridin-4-ylmethyl)carbamoyl)isophthalate and 14-azido-3,6,9,12-tetraoxatetradecan-1-amine. The solvent was removed under reduced pressure and the residue was purified by column chromatography (DCM:MeOH=100:1→to 100:2→100:3→100:5→100:7). After evaporation of solvents, the collected product containing fractions gave a pale orange oil (96 mg, 82% yield).

HRMS (ESI+) C₃₅H₅₃N₁₀O₁₁ [M+H]⁺ calc 789.3890, found 789.3868

¹H NMR (400 MHz, CDCl₃): δ 8.58-8.51 (m, 2H), 8.47-8.43 (m, 2H), 8.39-8.36 (m, 1H), 8.08-8.00 (m, 1H), 7.57-7.50 (m, 2H), 7.35-7.30 (m, 2H), 4.65 (d, J=5.9 Hz, 2H), 3.71-3.51 (m, 36H), 3.34-3.27 (m, 4H).

### 6.27.4. Synthesis of the Complex N¹,N³-bis(14-azido-3,6,9,12-tetraoxatetradecyl)-N⁵-(pyridin-4-ylmethyl)benzene-1,3,5-tricarboxamide-Pt(ethane-1,2-diamine)I]⁺ TFA⁻ (6aa)

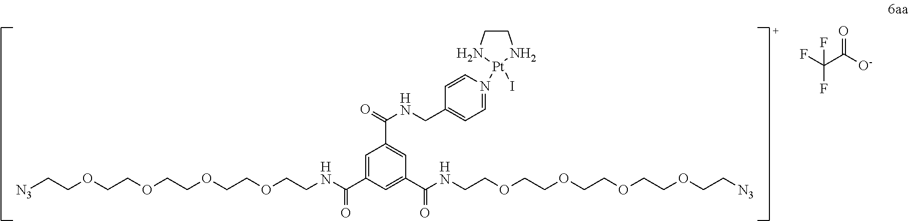

N¹,N³-Bis(14-azido-3,6,9,12-tetraoxatetradecyl)-N⁵-(pyridin-4-ylmethyl)benzene-1,3,5-tricarboxamide (L10) (bis-N₃-PEG₄-benzene-py; 39.5 mg, 50 μmol, 1.0 eq.) and Pt(ethane-1,2-diamine)I₂ (3a) (25.4 mg, 50 μmol, 1.0 eq.) were dissolved in dry DMF (500 μL) under argon atmosphere resulting in a homogeneous yellow mixture. The reaction mixture was stirred at 50° C. for 19 h, and the course of the reaction was followed by HPLC. Then, additional Pt(ethane-1,2-diamine)I₂ (3a) (25.4 mg, 50 μmol, 1.0 eq.) was added to the reaction mixture. The reaction mixture was stirred at 50° C. for 24 h, and the course of the reaction was followed by HPLC. Thereafter, additional Pt(ethane-1,2-diamine)I₂ (3a) (25.4 mg, 50 μmol, 1.0 eq.) was added to the reaction mixture. The reaction mixture was stirred at 50° C. for 24 h, and the course of the reaction was followed by HPLC. At this moment, the reaction mixture contained 98.1% product.

The reaction mixture was diluted with water (10 mL) and filtered through a paper filter to remove precipitated excessive Pt(ethane-1,2-diamine)I₂ (3a). The filtrate was applied to a column containing RP-C18 (LICHROPREP®, 15-25 μm; 500 mg, prewashed with MeOH (3 mL)). The run-out was discarded. The column was then washed subsequently with water/MeOH (9:1, 9 mL) and with water/MeOH (8:2, 5 mL). After that, the product was eluted with water/MeOH (2:8, 4 mL). HPLC analysis indicated that this fraction contained 99.6% product. This fraction was mixed with a NaI (13.2 mg) solution in water (1 mL). The mixture was further diluted with water (5 mL) and concentrated under reduced pressure. After been frozen, the mixture was lyophilized giving a yellow film (62.0 mg; corrected for the NaI content: 48.8 mg, 76.0% yield). The material was used to prepare a 5 mM solution in a 10 mM aqueous NaI solution; in this form the material was used and stored.

HRMS (ESI+) C₃₇H₆₀IN₁₂O₁₁¹⁹⁵Pt[M]⁺ calc 1170.3194, found 1170.3204

HPLC (Grace Alltima C18 5 μm column, 25×4.6 mm) indicated that the product was 99.0% pure (retention time 11.2 min; gradient: 20 to 100% MeCN/0.1% TFA in water/0.1% TFA in 20 min measured at a wavelength of 223 nm).

Comparison of conjugation efficiencies using different halide salts (an iodide, a bromide or a chloride releasing agent)

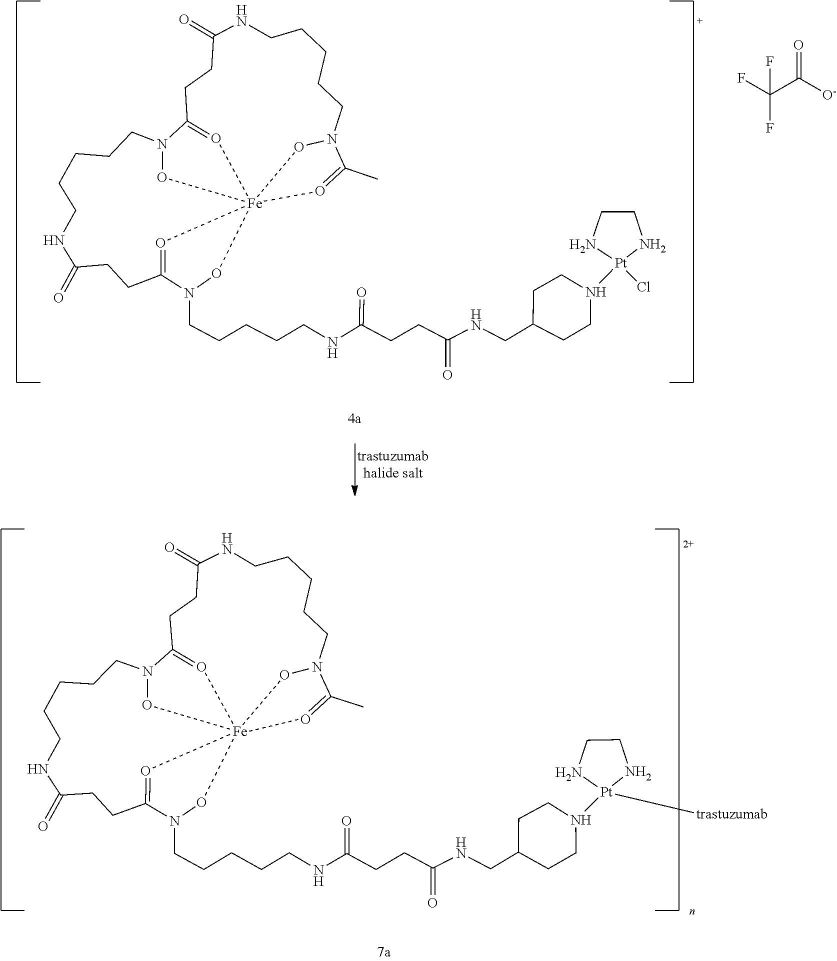

Trastuzumab (HERCEPTIN®; 35.5 μL, 21 mg/mL, 1.0 eq.), rebuffered from the pharmacy storage buffer to PBS by spin filtration, was diluted with water (15 μL), 200 mM HEPES buffer (6.15 pH 8.1) containing different halide salts (A: no additive, B: 2000 mM NaCl, pH 8.1; C: 500 mM NaBr, pH 8.1; D: 100 mM NaI, pH 8.1), and [PtCl((Fe)DFO-suc-pip)(ethane-1,2-diamine)]⁺ TFA⁻ (4a) (5.0 μL, 5 mM in 20 mM NaCl, 5.0 eq.) was added. The sample was incubated in a thermoshaker at 47° C. for 1 h, 2 h, 4 h, 6 h, and 24 h, followed by the addition of a solution of thiourea (61.7 μL, 20 mM in H₂O) and incubation at 37° C. for 30 min.

Conjugation efficiency was determined by SEC at 430 nm UV detection and was defined as the percentage of the (Fe)DFO chelate fraction bound to the protein in relation to the total (Fe)DFO amount, which also includes non-bound low MW fractions.

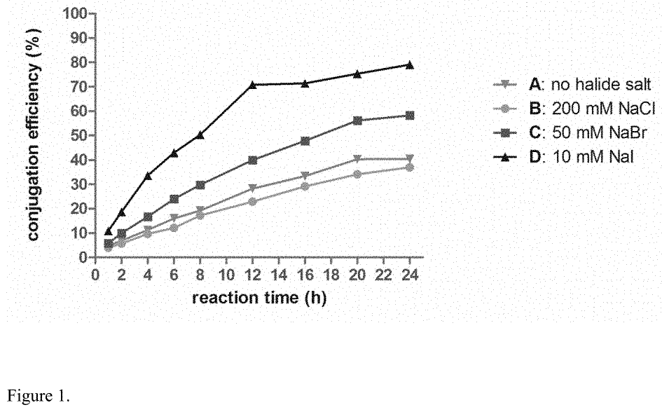

After 24 h conjugation time, the conjugation efficiencies were: 40% (A: no additional halide salt), 37% (B: 200 mM NaCl), 58% (C: 50 mM Br), and 79% (D: 10 mM NaI; FIG. 1).

Comparison of conjugation efficiencies using different monoclonal antibodies (cell binding moieties)

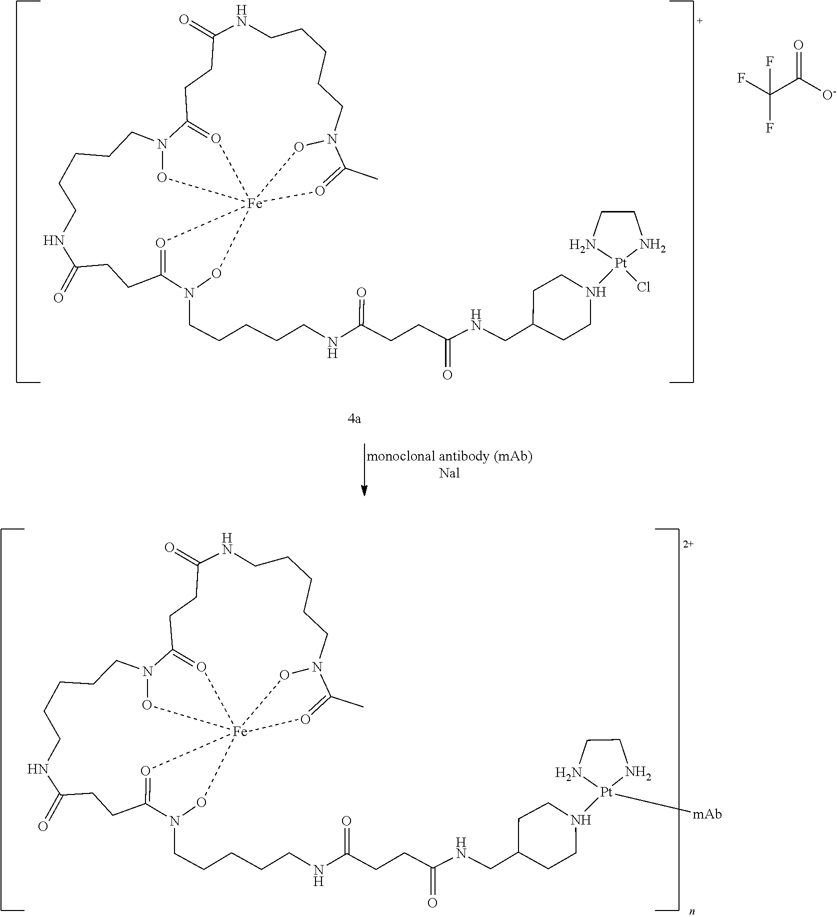

A monoclonal antibody, rebuffered from its formulation buffer to PBS by spin filtration (35.5 μL, 21 mg/mL, 1.0 eq.), was diluted with 200 mM HEPES buffer (6.15 pH 8.1) containing sodium iodide (100 mM NO; the final concentration of NaI in the reaction mixture was 10 mM), and [PtCl((Fe)DFO-suc-pip)(ethane-1,2-diamine)]⁺ TFA⁻ (4a) (20.0 L, 1.25 mM in 20 mM NaCl, 5.0 eq.) was added. The samples were incubated in a thermoshaker at 47° C. for 24 h, followed by the addition of a solution of thiourea (61.7 μL, 20 mM in H₂O) and incubation at 37° C. for 30 min.

Conjugation efficiency was determined by SEC at 430 nm UV detection and was defined as the percentage of the (Fe)DFO chelate fraction bound to the protein in relation to the total (Fe)DFO amount, which also includes non-bound low MW fractions.

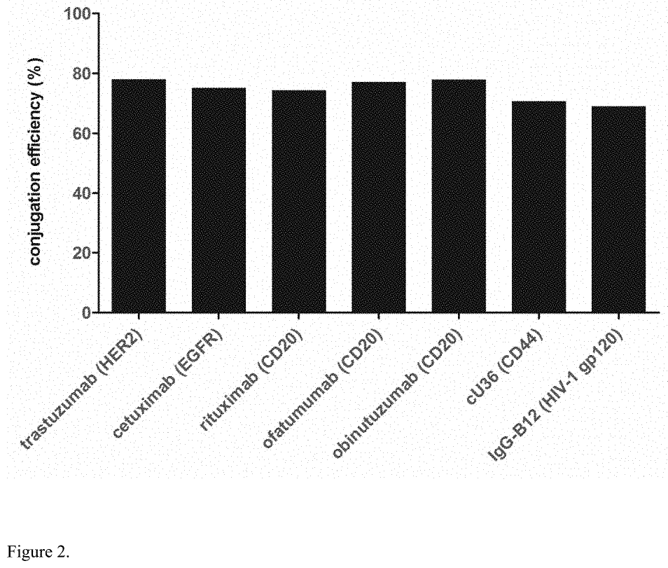

After 24 h conjugation time, the conjugation efficiencies were: 78% (trastuzumab), 75% (cetuximab), 74% (rituximab), 77% (ofatumumab), 78% (obinutuzumab), 71% (cU36), and 69% (IgG-B12); FIG. 2).

Comparison of conjugation efficiencies using different iodide or bromide salts (iodide or bromide releasing agents having different cations)

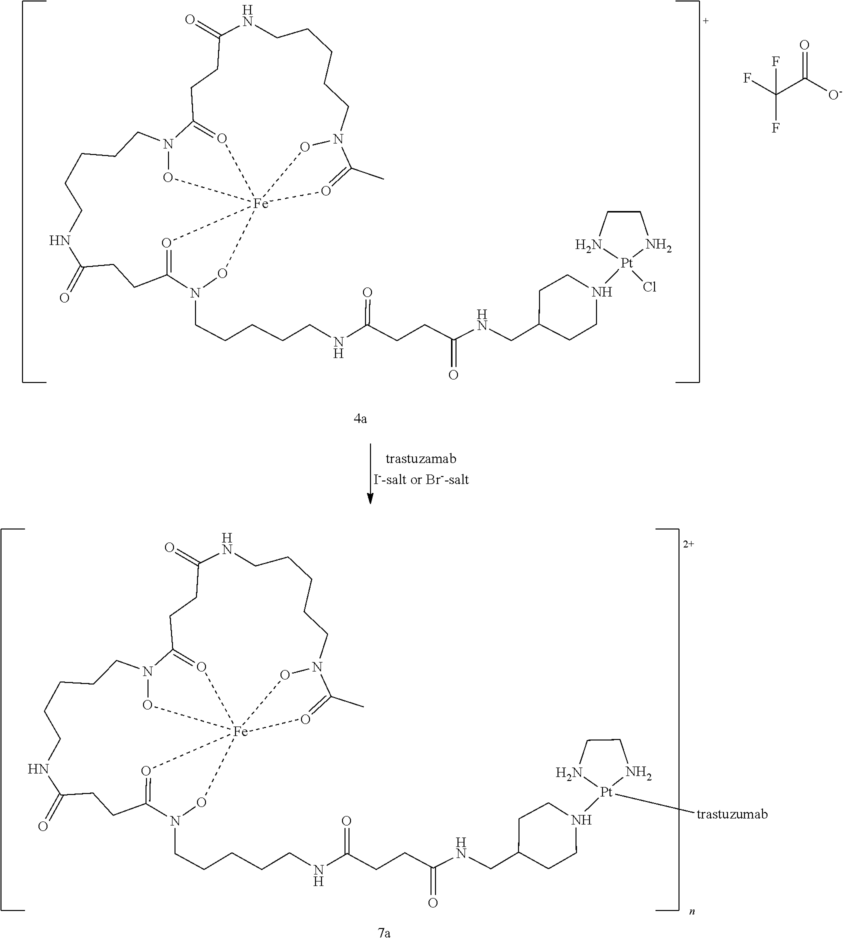

Trastuzumab (HERCEPTIN®; 35.5 L, 21 mg/mL, 1.0 eq.), rebuffered from its formulation buffer to PBS by spin filtration, was diluted with 200 mM HEPES buffer (6.15 pH 8.1) containing an iodide salt (100 mM I⁻-salt; the final concentration of I⁻ in the reaction mixture was 10-40 mM depending on the cation) or a bromide salt ((500 mM Br⁻-salt; the final concentration of Br⁻ in the reaction mixture was 50 mM)), and [PtCl((Fe)DFO-suc-pip)(ethane-1,2-diamine)]⁺ TFA⁻ (4a) (20.0 μL, 1.25 mM in 20 mM NaCl, 5.0 eq.) was added. The samples were incubated in a thermoshaker at 47° C. for 24 h, followed by the addition of a solution of thiourea (61.7 μL, 20 mM in H₂O) and incubation at 37° C. for 30 min.

Conjugation efficiency was determined by SEC at 430 nm UV detection and was defined as the percentage of the (Fe)DFO chelate fraction bound to the protein in relation to the total (Fe)DFO amount, which also includes non-bound low MW fractions.

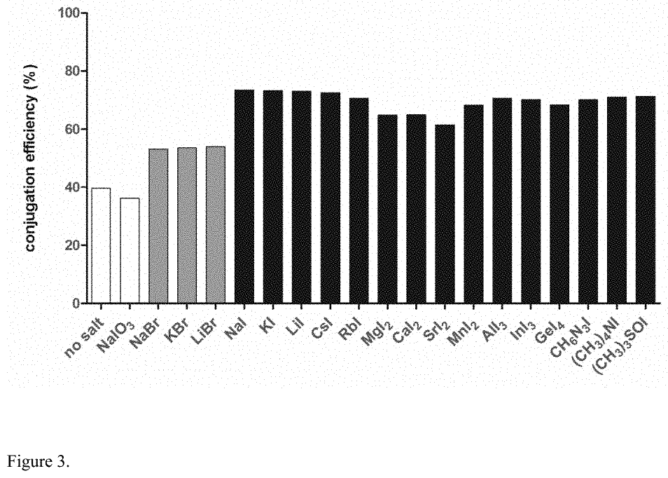

After 24 h conjugation time, the conjugation efficiencies were: 40% (no salt), 36% (NaIO₃ as a negative control), 53% (NaBr), 54% (KBr), 54% (LiBr), 73% (NaI), 73% (KI), 73% (LiI), 73% (CsI), 71% (RbI), 55% (NH₄I), 65% (MgI₂), 65% (CaI₂), 61% (SrI₂), 68% (MnI₂), 71% (AlI₃), 70% (InI₃), 68% (GeI₄), 70% (CH₆N₃I, guanidinium iodide), 71% ((CH₃)₄NI, tetramethylammonium iodide), 51% (C₇H₁₂NSOI, 5-(2-hydroxyethyl)-3,4-dimethylthiazol-3-ium iodide), and 71% ((CH₃)₃SOI, trimethylsulfoxonium iodide); FIG. 3).

Comparison of conjugation efficiencies at different pH values (using different buffers)

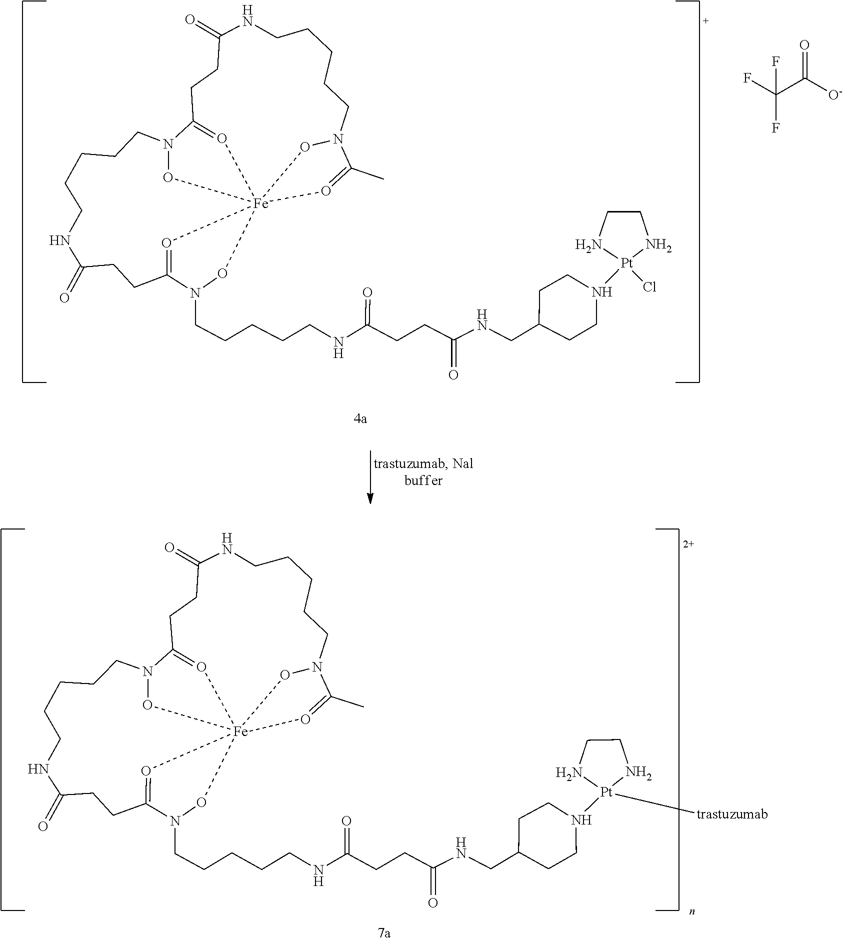

Trastuzumab (HERCEPTIN®; 35.5 μL, 21 mg/mL, 1.0 eq.), rebuffered from its formulation buffer to PBS by spin filtration, was diluted with a 200 mM buffer (6.15 μL, the final buffer concentration was 20 mM; pH was determined by a pH meter) containing NaI (100 mM; the final concentration of I⁻ in the reaction mixture was 10 mM), and [PtCl((Fe)DFO-suc-pip)(ethane-1,2-diamine)]⁺ TFA⁻ (4a) (20.0 μL, 1.25 mM in 20 mM NaCl, 5.0 eq.) was added. The samples were incubated in a thermoshaker at 47° C. for 24 h, followed by the addition of a solution of thiourea (61.7 μL, 20 mM in H₂O) and incubation at 37° C. for 30 min.

Conjugation efficiency was determined by SEC at 430 nm UV detection and was defined as the percentage of the (Fe)DFO chelate fraction bound to the protein in relation to the total (Fe)DFO amount, which also includes non-bound low MW fractions.

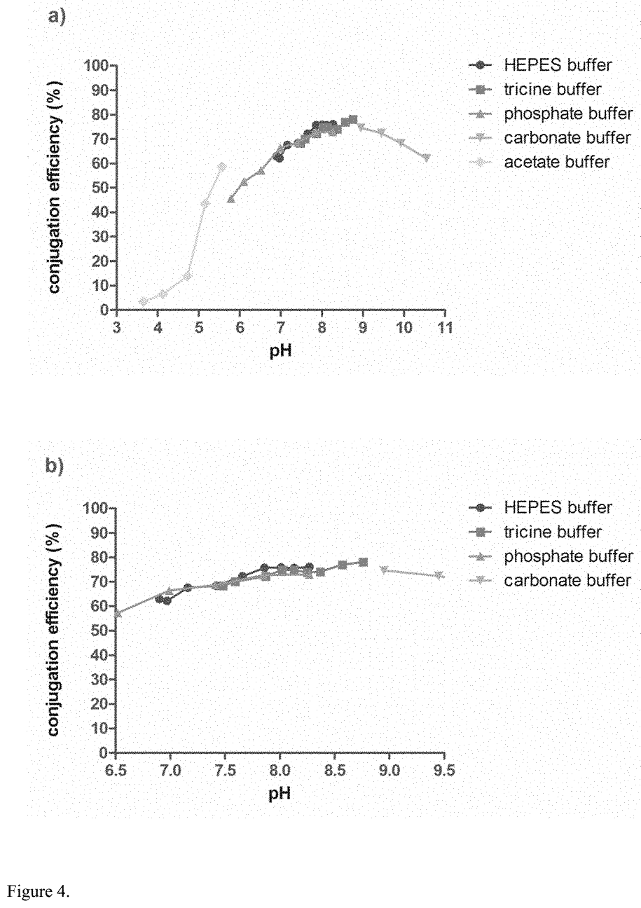

After 24 h conjugation time, the conjugation efficiencies were: 3% (acetate buffer, pH 3.65), 7% (acetate buffer, pH 4.13), 14% (acetate buffer, pH 4.73), 44% (acetate buffer, pH 5.16), 59% (acetate buffer, pH 5.57); 46% (phosphate buffer, pH 5.79), 53% (phosphate buffer, pH 6.10), 57% (phosphate buffer, pH 6.52), 66% (phosphate buffer, pH 6.99), 69% (phosphate buffer, pH 7.41), 73% (phosphate buffer, pH 7.85), 73% (phosphate buffer, pH 8.27); 63% (HEPES buffer, pH 6.90), 62% (HEPES buffer, pH 6.97), 68% (HEPES buffer, pH 7.16), 68% (HEPES buffer, pH 7.42), 72% (HEPES buffer, pH 7.66), 76% (HEPES buffer, pH 7.86), 76% (HEPES buffer, pH 8.01), 76% (HEPES buffer, pH 8.13), 76% (HEPES buffer, pH 8.27); 68% (tricine buffer, pH 7.48), 70% (tricine buffer, pH 7.59), 72% (tricine buffer, pH 7.87), 75% (tricine buffer, pH 8.02), 75% (tricine buffer, pH 8.13), 74% (tricine buffer, pH 8.25), 74% (tricine buffer, pH 8.37), 77% (tricine buffer, pH 8.57), 78% (tricine buffer, pH 8.76); 75% (carbonate buffer, pH 8.95), 72% (carbonate buffer, pH 9.45), 68% (carbonate buffer, pH 9.93), and 62% (carbonate buffer, pH 10.55); FIG. 4).

### Example 7: Examples of Trastuzumab-Lx Conjugates 7a-j

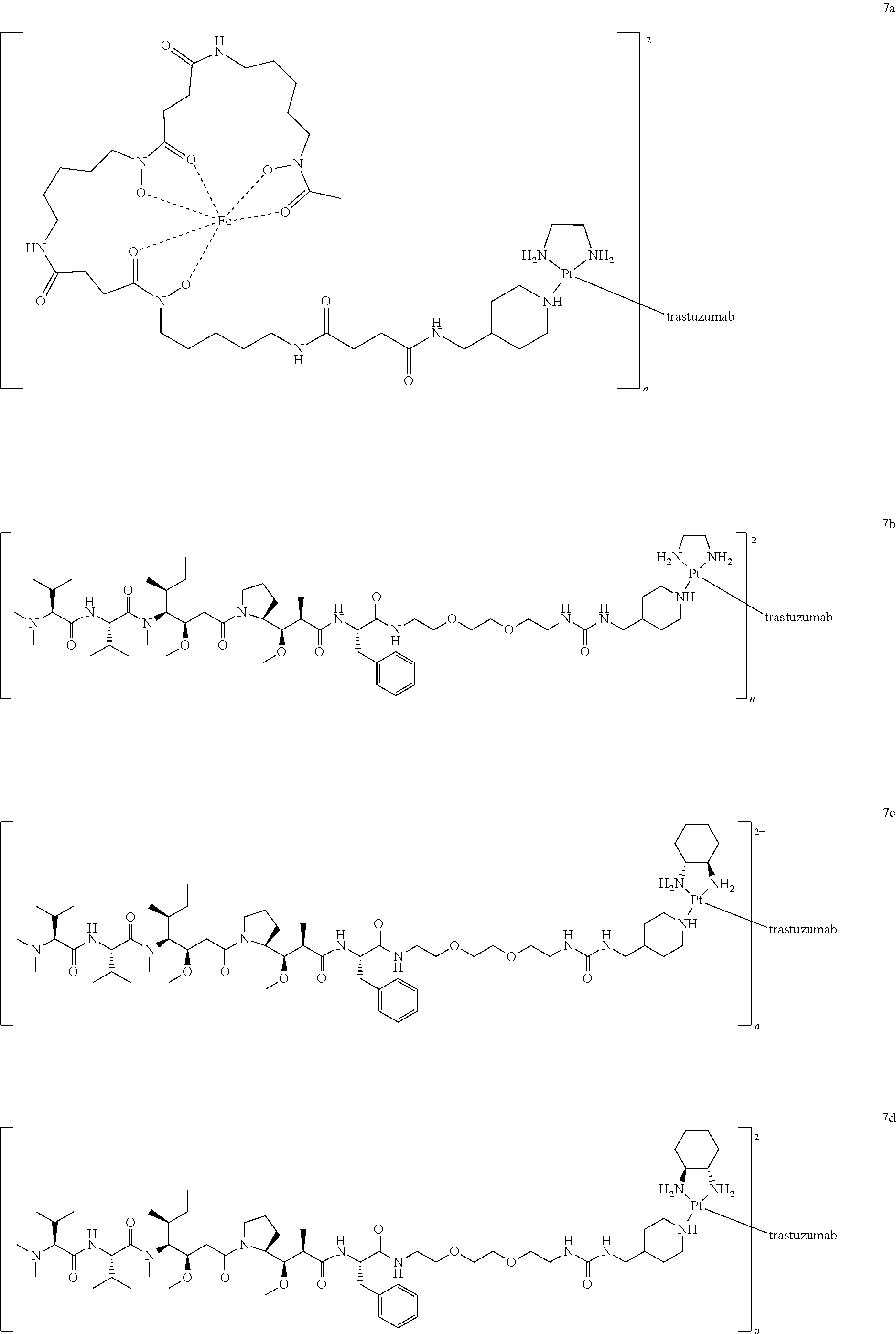

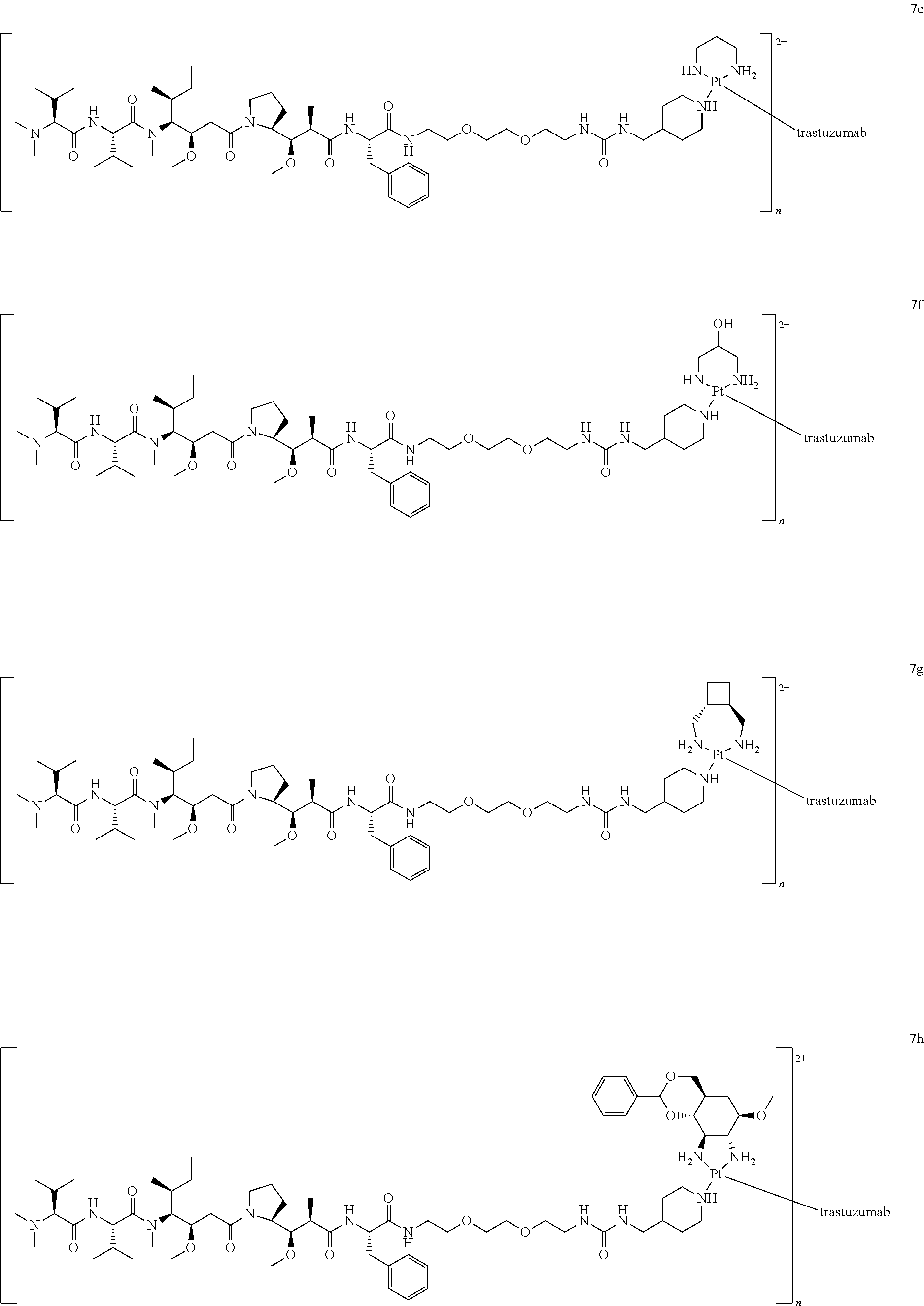

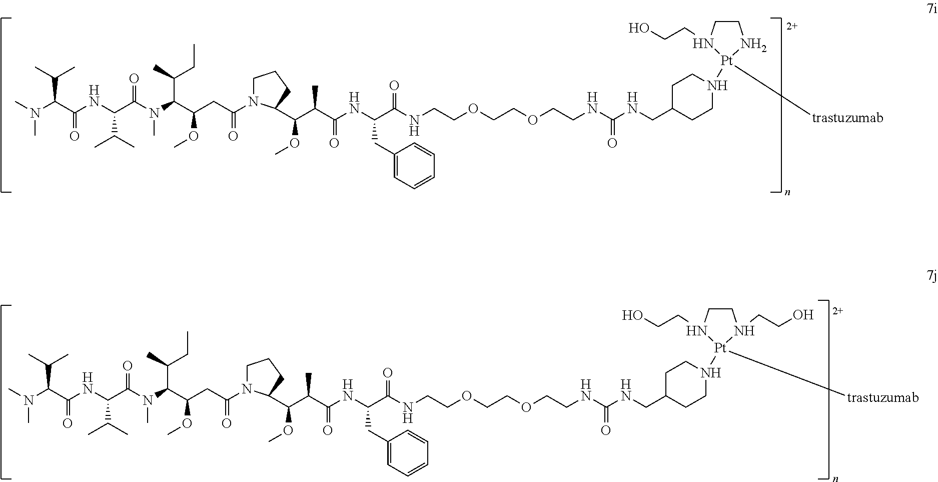

### 7.1. Synthesis and Analytical Characterization of the Bioconjugate trastuzumab-[Pt((Fe)DFO-suc-pip)(ethane-1,2-diamine)]n (7a); n=0-6

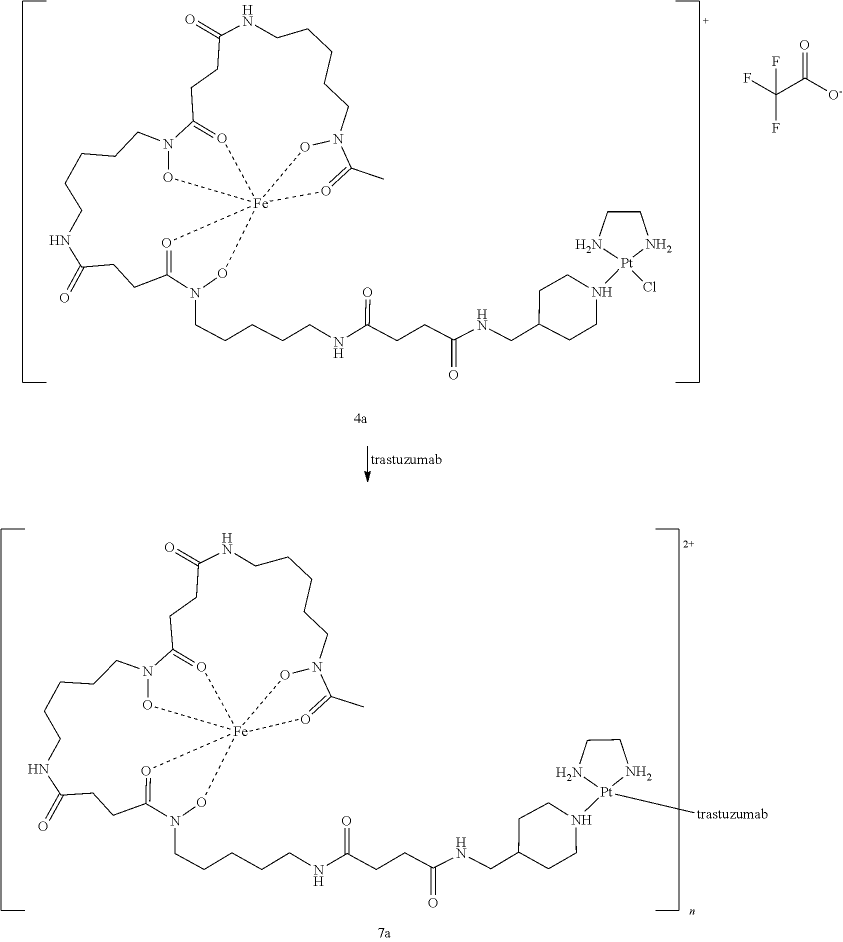

Trastuzumab (HERCEPTIN®; 35.5 μL, 21 mg/mL, 1.0 eq.), rebuffered from the pharmacy storage buffer to PBS by spin filtration, was diluted with 200 mM HEPES buffer (6.15 pH 8.1) containing 100 mM NaI, and [PtCl((Fe)DFO-suc-pip)(ethane-1,2-diamine)]⁺ TFA⁻ (4a) (20.0 μL, 825 μM in 20 mM NaCl, 3.3 eq.) was added. The sample was incubated in a thermoshaker at 47° C. for 24 h, followed by addition of a solution of thiourea (61.7 μL, 20 mM in H₂O) and incubation at 37° C. for 30 min. The conjugate was purified by PD-10 column (equilibrated with phosphate buffered saline), followed by spin filtration using 30 kD MWCO filters (washed 4× with PBS buffer), after which it was reconstituted and stored in PBS buffer.

The antibody integrity was controlled by SEC (after removal of Fe(III) using EDTA): 96.8% monomer. SEC-MS analysis was performed after purification of the conjugate 7a to determine the DAR: DAR=2.18 (corresponds to 66% conjugation efficiency). The complex distribution on the fragments of trastuzumab was determined by SDS-PAGE/phosphorimager analysis: % Hc=87%, % Lc=13%, % F(ab′)2=30%.

### 7.2. Synthesis and Analytical Characterization of the Bioconjugate trastuzumab-[Pt(auristatin F-(4-(12-amino-3-oxo-7,10-dioxa-2,4-diazadodecyl)piperidine))(ethane-1,2-diamine)]ₙ (7b)

n=0-6

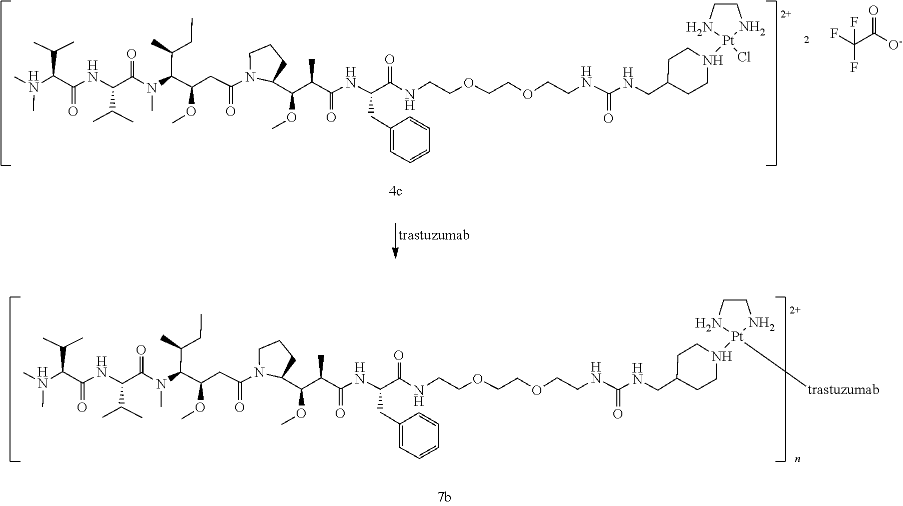

Trastuzumab (HERCEPTIN®; 35.5 μL, 21 mg/mL, 1.0 eq.), rebuffered from the pharmacy storage buffer to PBS by spin filtration, was diluted with 200 mM HEPES buffer (6.15 μL, pH 8.1) containing 100 mM of NaI solution, and Pt(auristatin F-(4-(12-amino-3-oxo-7,10-dioxa-2,4-diazadodecyl)piperidine))C₁(ethane-1,2-diamine) (4c) (20.0 μL, 825 μM in 20 mM NaCl, 3.3 eq.) was added. The sample was incubated in a thermoshaker at 47° C. for 24 h, followed by the addition of a solution of thiourea (61.7 μL, 20 mM in H₂O) and incubation at 37° C. for 30 min. The conjugate was purified by PD-10 column (equilibrated with phosphate buffered saline), followed by spin filtration using 30 kD MWCO filters (washed 4× with PBS buffer), after which it was reconstituted and stored in PBS buffer.

The antibody integrity was controlled by SEC: 98.2% monomer. SEC-MS analysis was performed after purification of the conjugate 7b to determine the DAR and the complex distribution on the fragments of trastuzumab: DAR=2.81 (corresponds to 85% conjugation efficiency), % Hc=87%, % Lc=13%, % F(ab′)2=22%, % Fab=15%, % Fc=85%.

### 7.3. Synthesis and Analytical Characterization of the Bioconjugate trastuzumab-[Pt(auristatin F-(4-(12-amino-3-oxo-7,10-dioxa-2,4-diazadodecyl)piperidine))((1R,2R)-(−)-1,2-diaminocyclohexane)]ₙ (7c)

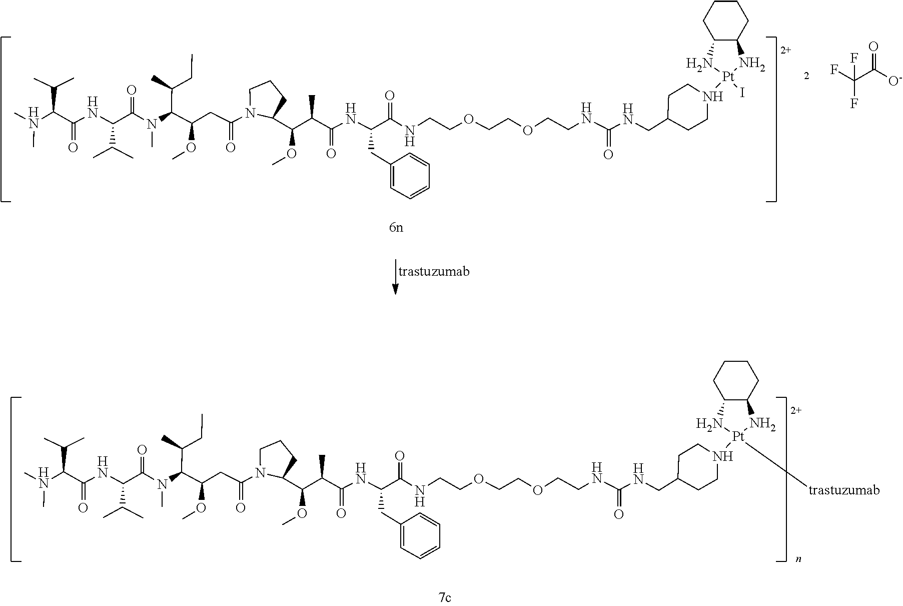

Trastuzumab (HERCEPTIN®; 71 μL, 21 mg/mL, 1.0 eq.), rebuffered from the pharmacy storage buffer to PBS by spin filtration, was diluted with MilliQ water (33.4 μL) and with 200 mM HEPES buffer (12.3 pH 8.1) containing 100 mM of NaI solution, and Pt(auristatin F-(4-(12-amino-3-oxo-7,10-dioxa-2,4-diazadodecyl)piperidine))I((1R,2R)-(−)-1,2-diaminocyclohexane) (6n) (6.6 μL, 5 mM in 20 mM NaI, 3.3 eq.) was added. The sample was incubated in a thermoshaker at 47° C. for 24 h, followed by the addition of a solution of thiourea (123.3 μL, 20 mM in H₂O) and incubation at 37° C. for 30 min. The conjugate was purified by PD-10 column (equilibrated with phosphate buffered saline), followed by spin filtration using 30 kD MWCO filters (washed 4× with PBS buffer), after which it was reconstituted and stored in PBS buffer.

The antibody integrity was controlled by SEC: 98.1% monomer. SEC-MS analysis was performed after purification of the conjugate 7c to determine the DAR: DAR=3.3 (corresponds to a quantitative conjugation efficiency).

### 7.4. Synthesis and Analytical Characterization of the Bioconjugate trastuzumab-[Pt(auristatin F-(4-(12-amino-3-oxo-7,10-dioxa-2,4-diazadodecyl)piperidine))((1S,2S)-(−)-1,2-diaminocyclohexane)]ₙ (7d)

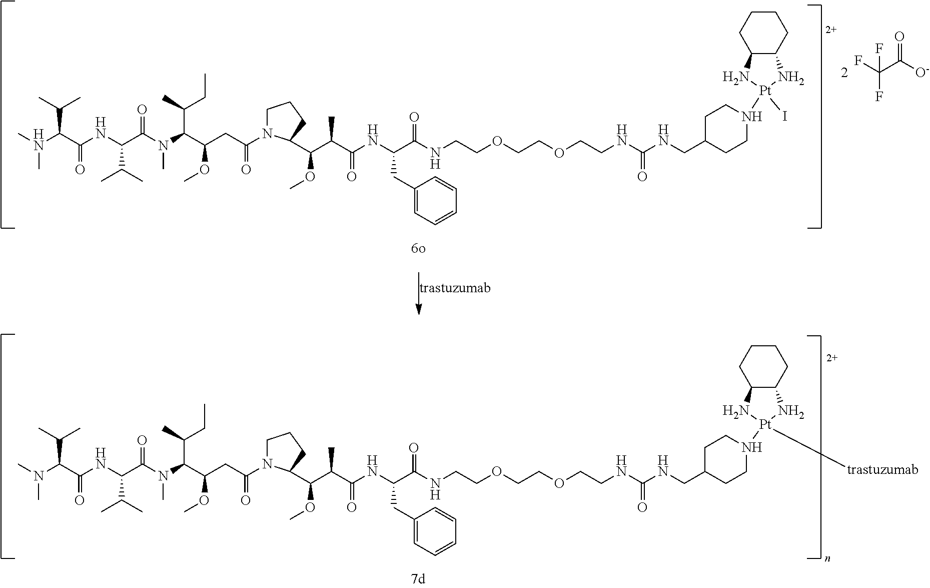

Trastuzumab (HERCEPTIN®; 71 μL, 21 mg/mL, 1.0 eq.), rebuffered from the pharmacy storage buffer to PBS by spin filtration, was diluted with MilliQ water (34 μL) and with 200 mM HEPES buffer (12.3 pH 8.1) containing 100 mM of NaI solution, and Pt(auristatin F-(4-(12-amino-3-oxo-7,10-dioxa-2,4-diazadodecyl)piperidine))I(((1S,2S)-(−)-1,2-diaminocyclohexane)) (6o) (6 μL, 5 mM in 20 mM NaI, 3.0 eq.) was added. The sample was incubated in a thermoshaker at 47° C. for 24 h, followed by the addition of a solution of thiourea (123.3 μL, 20 mM in H₂O) and incubation at 37° C. for 30 min. The conjugate was purified by PD-10 column (equilibrated with phosphate buffered saline), followed by spin filtration using 30 kD MWCO filters (washed 4× with PBS buffer), after which it was reconstituted and stored in PBS buffer.

The antibody integrity was controlled by SEC: 98.5% monomer. SEC-MS analysis was performed after purification of the conjugate 7d to determine the DAR: DAR=3.0 (corresponds to 91% conjugation efficiency).

### 7.5. Synthesis and Analytical Characterization of the Bioconjugate trastuzumab-[Pt(auristatin F-(4-(12-amino-3-oxo-7,10-dioxa-2,4-diazadodecyl)piperidine))(propane-1,3-diamine)]ₙ (7e)

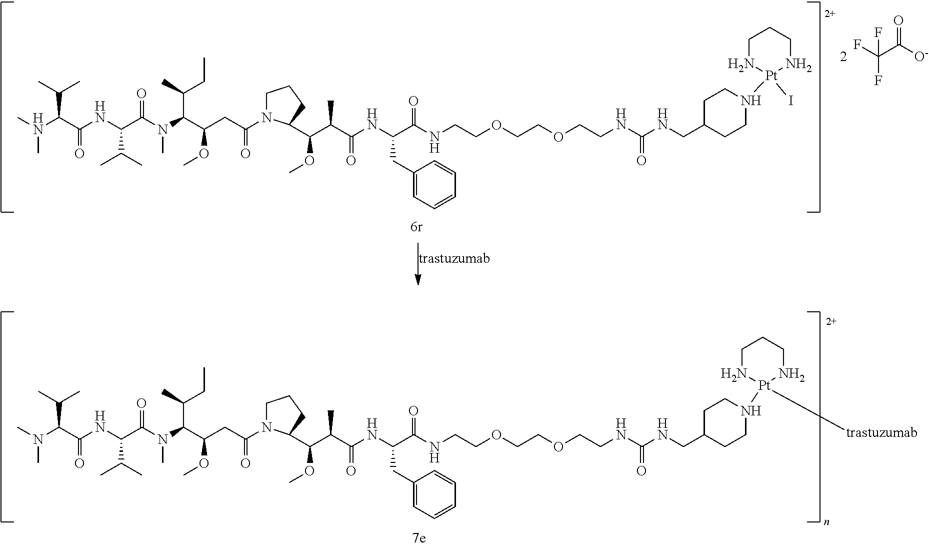

Trastuzumab (HERCEPTIN®; 71 μL, 21 mg/mL, 1.0 eq.), rebuffered from the pharmacy storage buffer to PBS by spin filtration, was diluted with MilliQ water (34 μL) and with 200 mM HEPES buffer (12.3 pH 8.1) containing 100 mM of NaI solution, and Pt(auristatin F-(4-(12-amino-3-oxo-7,10-dioxa-2,4-diazadodecyl)piperidine))I(propane-1,3-diamine) (6r) (6 μL, 5 mM in 20 mM NaI, 3.0 eq.) was added. The sample was incubated in a thermoshaker at 47° C. for 24 h, followed by the addition of a solution of thiourea (123.3 μL, 20 mM in H₂O) and incubation at 37° C. for 30 min. The conjugate was purified by PD-10 column (equilibrated with phosphate buffered saline), followed by spin filtration using 30 kD MWCO filters (washed 4× with PBS buffer), after which it was reconstituted and stored in PBS buffer.

The antibody integrity was controlled by SEC: 96.3% monomer. SEC-MS analysis was performed after purification of the conjugate 7e to determine the DAR: DAR=2.7 (corresponds to 90% conjugation efficiency).

### 7.6. Synthesis and Analytical Characterization of the Bioconjugate trastuzumab-[Pt(auristatin F-(4-(12-amino-3-oxo-7,10-dioxa-2,4-diazadodecyl)piperidine))(1,3-diaminopropan-2-ol)]n (7f)

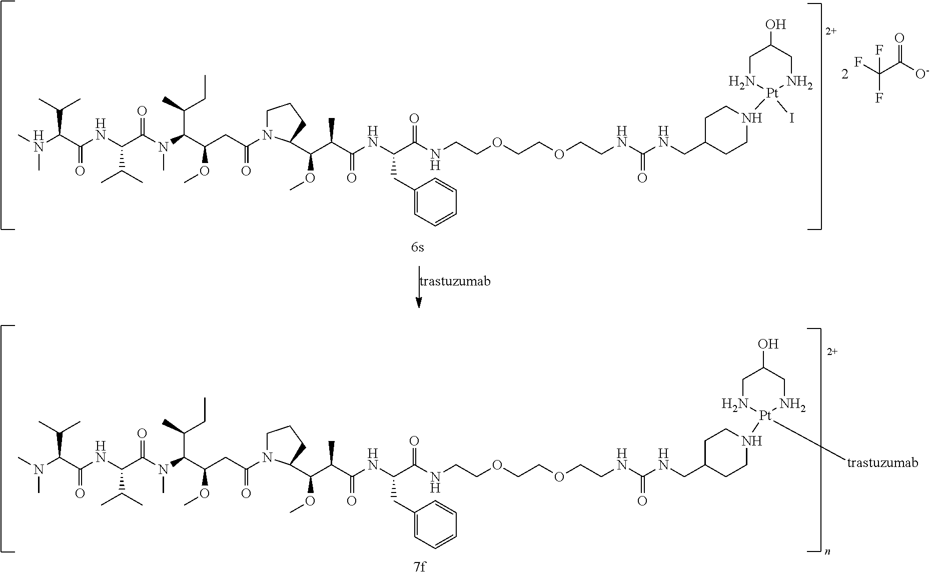

Trastuzumab (HERCEPTIN®; 238 μL, 21 mg/mL, 1.0 eq.), rebuffered from the pharmacy storage buffer to PBS by spin filtration, was diluted with MilliQ water (105.6 μL) and 200 mM HEPES buffer (41.2 pH 8.1) containing 100 mM of NaI solution, and Pt(auristatin F-(4-(12-amino-3-oxo-7,10-dioxa-2,4-diazadodecyl)piperidine))I(1,3-diaminopropan-2-ol) (6s) (28.5 μL, 5 mM in 20 mM NaI, 4.2 eq.) was added. The sample was incubated in a thermoshaker at 47° C. for 2 h, followed by the addition of a solution of thiourea (411 μL, 20 mM in H₂O) and incubation at 37° C. for 30 min. The conjugate was purified by PD-10 column (equilibrated with phosphate buffered saline), followed by spin filtration using 30 kD MWCO filters (washed 4× with PBS buffer), after which it was reconstituted and stored in PBS buffer.

The antibody integrity was controlled by SEC: 98.9% monomer. SEC-MS analysis was performed after purification of the conjugate 7f to determine the DAR: DAR=2.7 (corresponds to 64% conjugation efficiency).

### 7.7. Synthesis and Analytical Characterization of the Bioconjugate trastuzumab-[Pt(auristatin F-(4-(12-amino-3-oxo-7,10-dioxa-2,4-diazadodecyl)piperidine))((1R,2R)-cyclobutane-1,2-diyl)dimethanamine)]ₙ (7g)

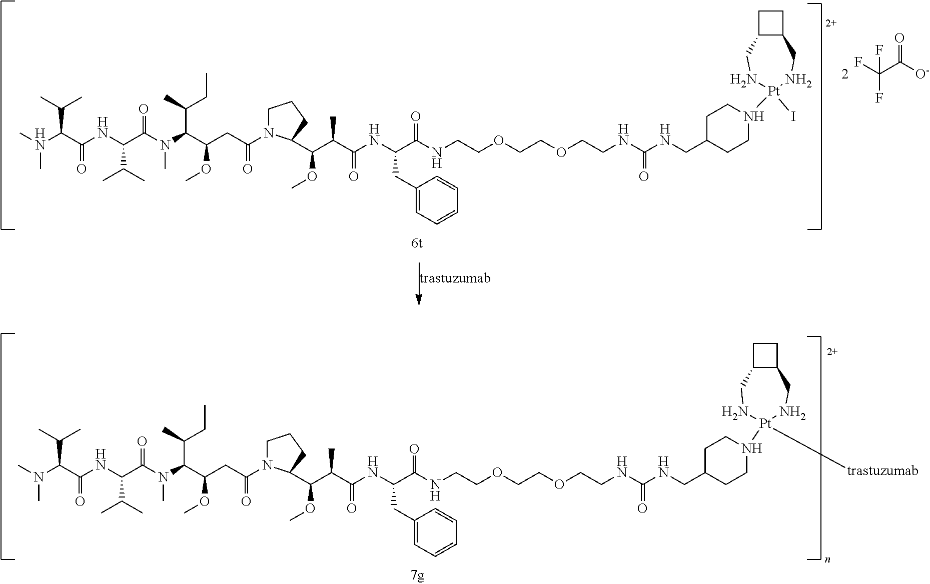

Trastuzumab (HERCEPTIN®; 71 μL, 21 mg/mL, 1.0 eq.), rebuffered from the pharmacy storage buffer to PBS by spin filtration, was diluted with MilliQ water (33.4 μL) and with 200 mM HEPES buffer (12.3 pH 8.1) containing 100 mM of NaI solution, and Pt(auristatin F-(4-(12-amino-3-oxo-7,10-dioxa-2,4-diazadodecyl)piperidine))I((1R,2R)-cyclobutane-1,2-diyl)dimethanamine) (6t) (6.6 μL, 5 mM in 20 mM NaI, 3.3 eq.) was added. The sample was incubated in a thermoshaker at 47° C. for 24 h, followed by the addition of a solution of thiourea (123.3 μL, 20 mM in H₂O) and incubation at 37° C. for 30 min. The conjugate was purified by PD-10 column (equilibrated with phosphate buffered saline), followed by spin filtration using 30 kD MWCO filters (washed 4× with PBS buffer), after which it was reconstituted and stored in PBS buffer.

The antibody integrity was controlled by SEC: 98.0% monomer. SEC-MS analysis was performed after purification of the conjugate 7g to determine the DAR: DAR=3.0 (corresponds to 91% conjugation efficiency).

### 7.8. Synthesis and Analytical Characterization of the Bioconjugate trastuzumab-[Pt(auristatin F-(4-(12-amino-3-oxo-7,10-dioxa-2,4-diazadodecyl)piperidine))((4aR,6R,7R,8R,8aS)-6-methoxy-2-phenylhexahydropyrano[3,2-d][1,3]dioxine-7,8-diamine]ₙ (7h)

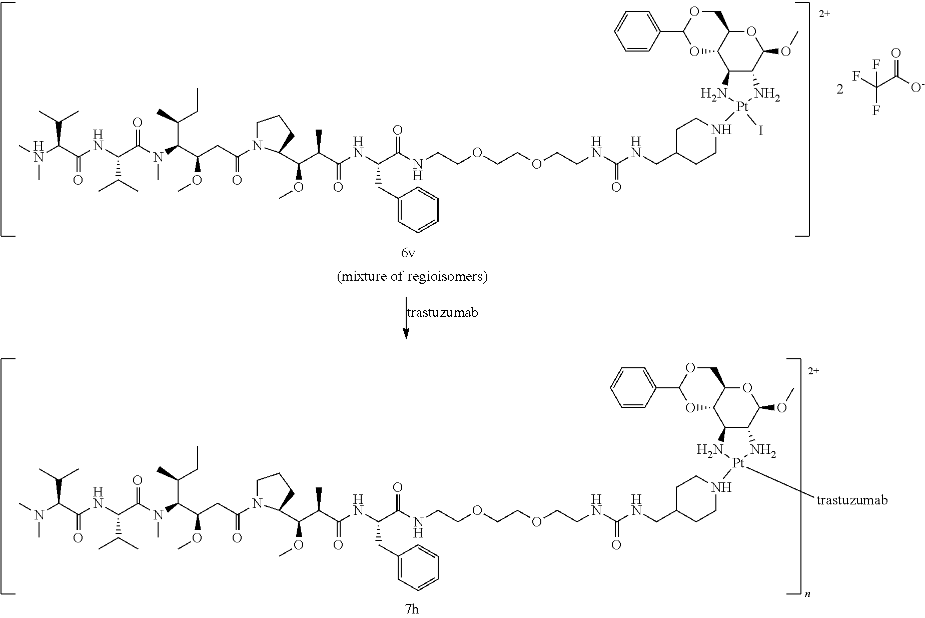

Trastuzumab (HERCEPTIN®; 71 μL, 21 mg/mL, 1.0 eq.), rebuffered from the pharmacy storage buffer to PBS by spin filtration, was diluted with MilliQ water (34 μL) and with 200 mM HEPES buffer (12.3 pH 8.1) containing 100 mM of NaI solution, and Pt(auristatin F-(4-(12-amino-3-oxo-7,10-dioxa-2,4-diazadodecyl)piperidine))I((4aR,6R,7R,8R,8aS)-6-methoxy-2-phenylhexahydropyrano[3,2-d][1,3]dioxine-7,8-diamine) (6v) (6 μL, 5 mM in 20 mM NaI, 3.0 eq.) was added. The sample was incubated in a thermoshaker at 47° C. for 24 h, followed by the addition of a solution of thiourea (123.3 μL, 20 mM in H₂O) and incubation at 37° C. for 30 min. The conjugate was purified by PD-10 column (equilibrated with phosphate buffered saline), followed by spin filtration using 30 kD MWCO filters (washed 4× with PBS buffer), after which it was reconstituted and stored in PBS buffer.

The antibody integrity was controlled by SEC: 96.7% monomer. SEC-MS analysis was performed after purification of the conjugate 7h to determine the DAR: DAR=2.2 (corresponds to 73% conjugation efficiency).

### 7.9. Synthesis and Analytical Characterization of the Bioconjugate trastuzumab-[Pt(auristatin F-(4-(12-amino-3-oxo-7,10-dioxa-2,4-diazadodecyl)piperidine))(2-((2-aminoethyl)amino)ethan-1-ol]n (7i)

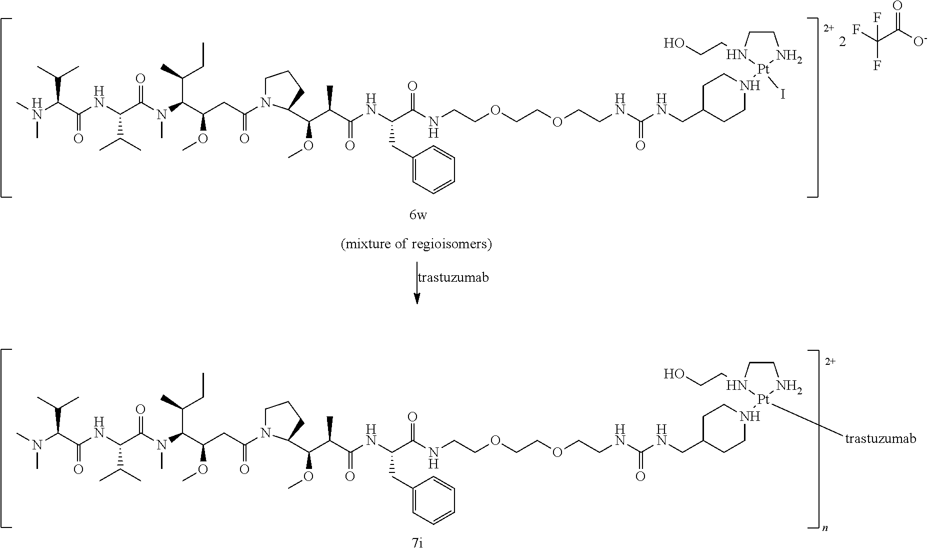

Trastuzumab (HERCEPTIN®; 71 μL, 21 mg/mL, 1.0 eq.), rebuffered from the pharmacy storage buffer to PBS by spin filtration, was diluted with MilliQ water (34 μL) and with 200 mM HEPES buffer (12.3 pH 8.1) containing 100 mM of NaI solution, and Pt(auristatin F-(4-(12-amino-3-oxo-7,10-dioxa-2,4-diazadodecyl)piperidine))I(2-(2-aminoethyl)amino)ethan-1-ol) (6w) (6 μL, 5 mM in 20 mM NaI, 3.0 eq.) was added. The sample was incubated in a thermoshaker at 47° C. for 24 h, followed by the addition of a solution of thiourea (123.3 μL, 20 mM in H₂O) and incubation at 37° C. for 30 min. The conjugate was purified by PD-10 column (equilibrated with phosphate buffered saline), followed by spin filtration using 30 kD MWCO filters (washed 4× with PBS buffer), after which it was reconstituted and stored in PBS buffer.

The antibody integrity was controlled by SEC: 98.4% monomer. SEC-MS analysis was performed after purification of the conjugate 7i to determine the DAR: DAR=2.8 (corresponds to 93% conjugation efficiency).

### 7.10. Synthesis and Analytical Characterization of the Bioconjugate trastuzumab-[Pt(auristatin F-(4-(12-amino-3-oxo-7,10-dioxa-2,4-diazadodecyl)piperidine))(2,2′-(ethane-1,2-diylbis(azanediyl))bis(ethan-1-ol)]n (7j)

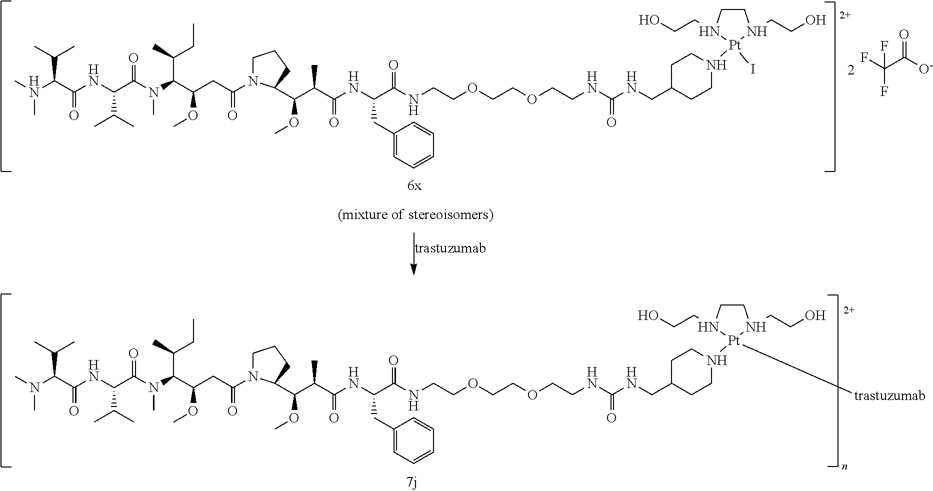

Trastuzumab (HERCEPTIN®; 71 μL, 21 mg/mL, 1.0 eq.), rebuffered from the pharmacy storage buffer to PBS by spin filtration, was diluted with MilliQ water (34 μL) and with 200 mM HEPES buffer (12.3 pH 8.1) containing 100 mM of NaI solution, and Pt(auristatin F-(4-(12-amino-3-oxo-7,10-dioxa-2,4-diazadodecyl)piperidine))I(2,2′-(ethane-1,2-diylbis(azanediyl))bis(ethan-1-ol)) (6x) (6 μL, 5 mM in 20 mM NaI, 3.0 eq.) was added. The sample was incubated in a thermoshaker at 47° C. for 24 h, followed by the addition of a solution of thiourea (123.3 μL, 20 mM in H₂O) and incubation at 37° C. for 30 min. The conjugate was purified by PD-10 column (equilibrated with phosphate buffered saline), followed by spin filtration using 30 kD MWCO filters (washed 4× with PBS buffer), after which it was reconstituted and stored in PBS buffer.

The antibody integrity was controlled by SEC: 98.3% monomer. SEC-MS analysis was performed after purification of the conjugate 7j to determine the DAR: DAR=2.6 (corresponds to 87% conjugation efficiency).

### Example 8: Examples of Azide-Bearing Trastuzumab-Lx Conjugates 8a-b Obtained from the “Semi-Final” Compounds (SFMs) 6z and 6Aa for Use in the Copper-Free Click Chemistry

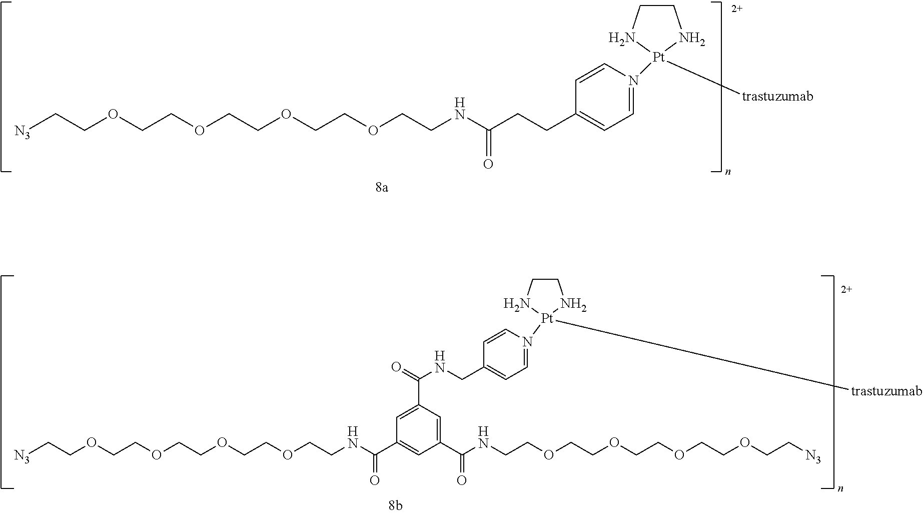

### 8.1. Synthesis and Analytical Characterization of the Bioconjugate trastuzumab-[Pt(N₃-PEG₄-py)(ethane-1,2-diamine)]n (8a)

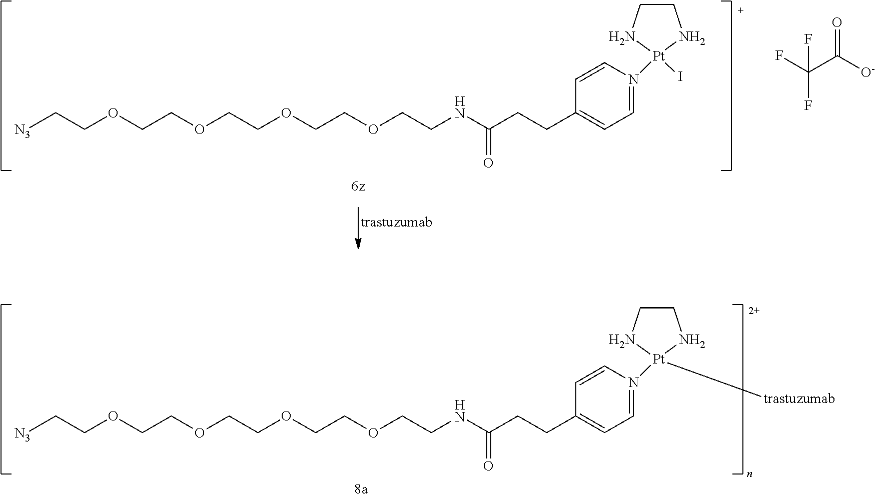

Trastuzumab (HERCEPTIN®; 238 μL, 21 mg/mL, 5.0 mg, 33 nmol, 1.0 eq.), rebuffered from the pharmacy storage buffer to PBS by spin filtration, was diluted with 200 mM HEPES buffer (41.2 μL, pH 8.1) containing 100 mM of NaI solution, and [N-(14-azido-3,6,9,12-tetraoxatetradecyl)-3-(pyridin-4-yl)propanamide-Pt(ethane-1,2-diamine)I]⁺ TFA⁻ (6z) (21.8 μL, 5 mM in 10 mM NaI, 109 nmol, 3.3 eq.) was added. The sample was further diluted with milliQ water (112.2 μL) and incubated in a thermoshaker at 47° C. for 24 h, followed by the addition of a solution of thiourea (413 μL, 20 mM in H₂O) and incubation at 37° C. for 30 min. The conjugate was purified by PD-10 column (equilibrated with phosphate buffered saline), followed by spin filtration using 30 kD MWCO filters (washed 1× with PBS buffer), after which it was reconstituted and stored in PBS buffer.

### 8.2. Synthesis and Analytical Characterization of the bioconjugate trastuzumab-[Pt(N¹,N³-bis(14-azido-3,6,9,12-tetraoxatetradecyl)-N⁵-(pyridin-4-ylmethyl)benzene-1,3,5-tricarboxamide)(ethane-1,2-diamine)]ₙ (8b)

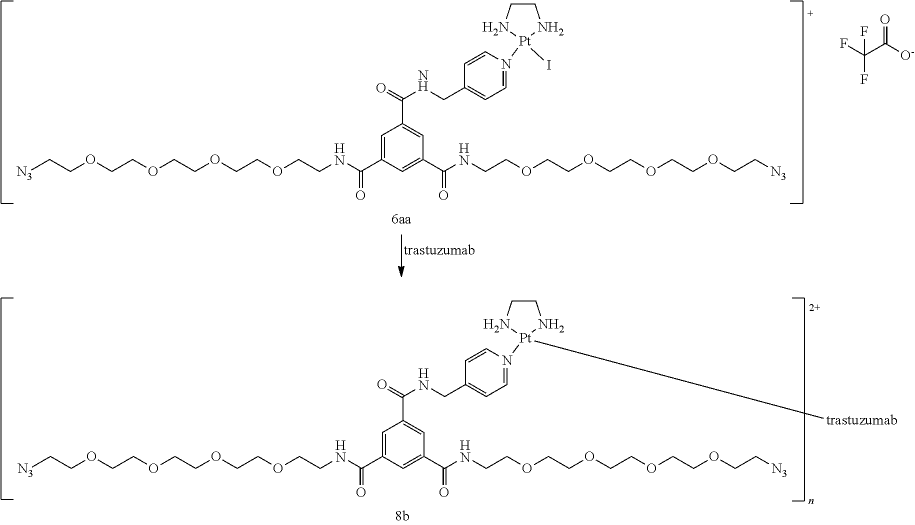

Trastuzumab (HERCEPTIN®; 238 μL, 21 mg/mL, 5.0 mg, 33 nmol, 1.0 eq.), rebuffered from the pharmacy storage buffer to PBS by spin filtration, was diluted with 200 mM HEPES buffer (41.2 pH 8.1) containing 100 mM of NaI solution, and [N¹,N³-bis(14-azido-3,6,9,12-tetraoxatetradecyl-Pt(ethane-1,2-diamine)I]⁺ TFA⁻ (6aa) (21.8 μL, 5 mM in 10 mM NaI, 109 nmol, 3.3 eq.) was added. The sample was further diluted with milliQ water (112.2 μL) and incubated in a thermoshaker at 47° C. for 24 h, followed by the addition of a solution of thiourea (413 μL, 20 mM in H₂O) and incubation at 37° C. for 30 min. The conjugate was purified by PD-10 column (equilibrated with phosphate buffered saline), followed by spin filtration using 30 kD MWCO filters (washed 1× with PBS buffer), after which it was reconstituted and stored in PBS buffer.

### Example 9: Examples of Trastuzumab-Lx Conjugates 9a-f Obtained from the Conjugate 8a Via the Copper-Free Click Chemistry

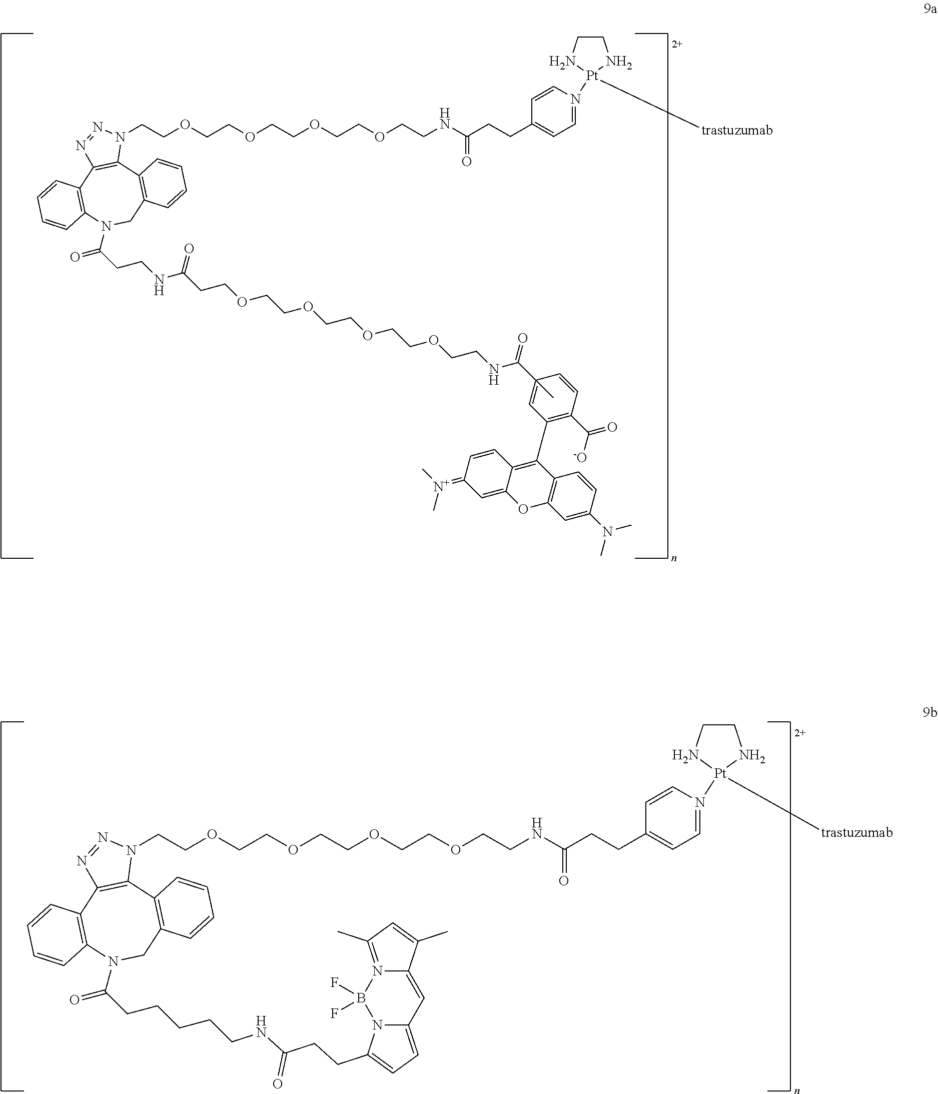

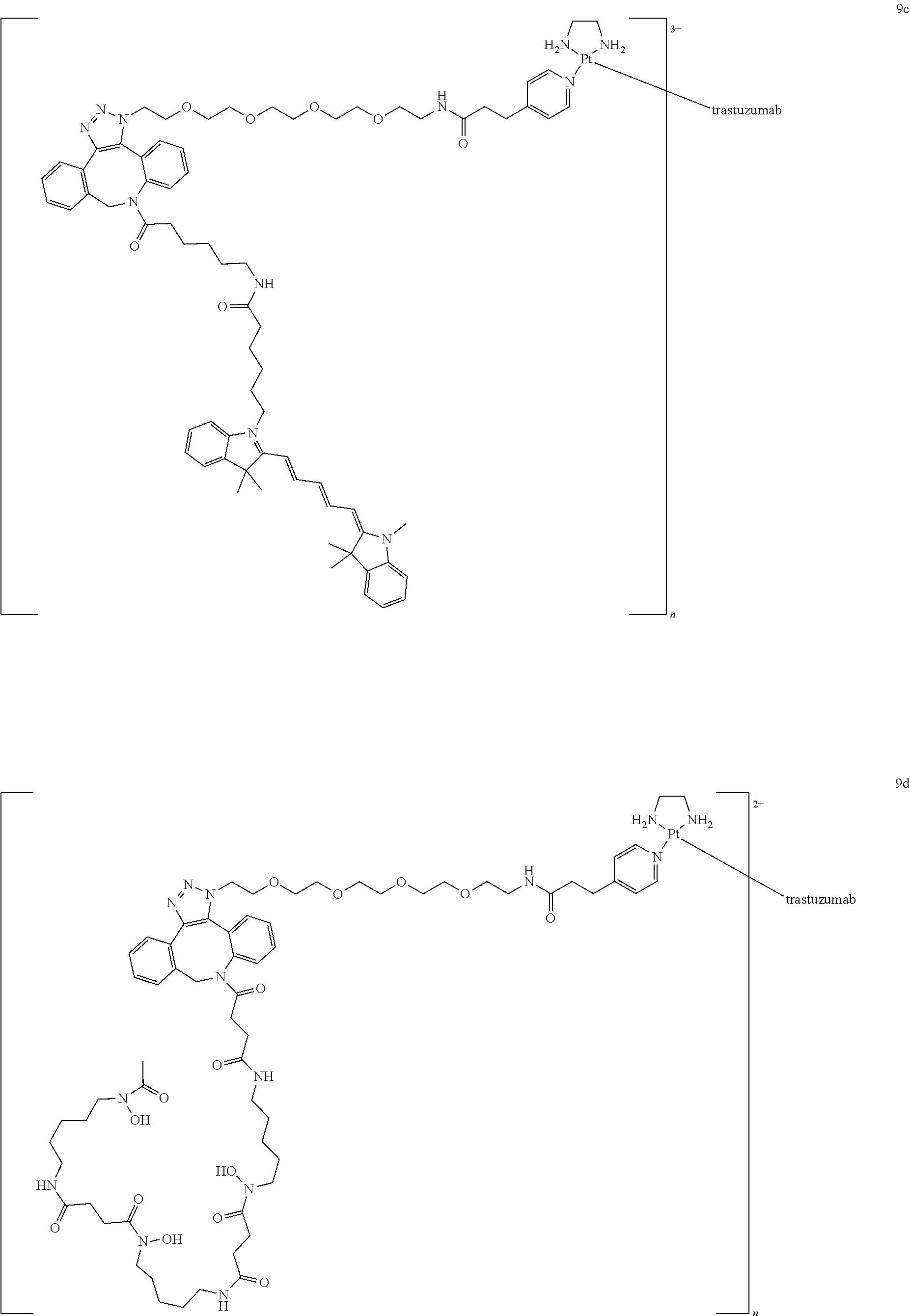

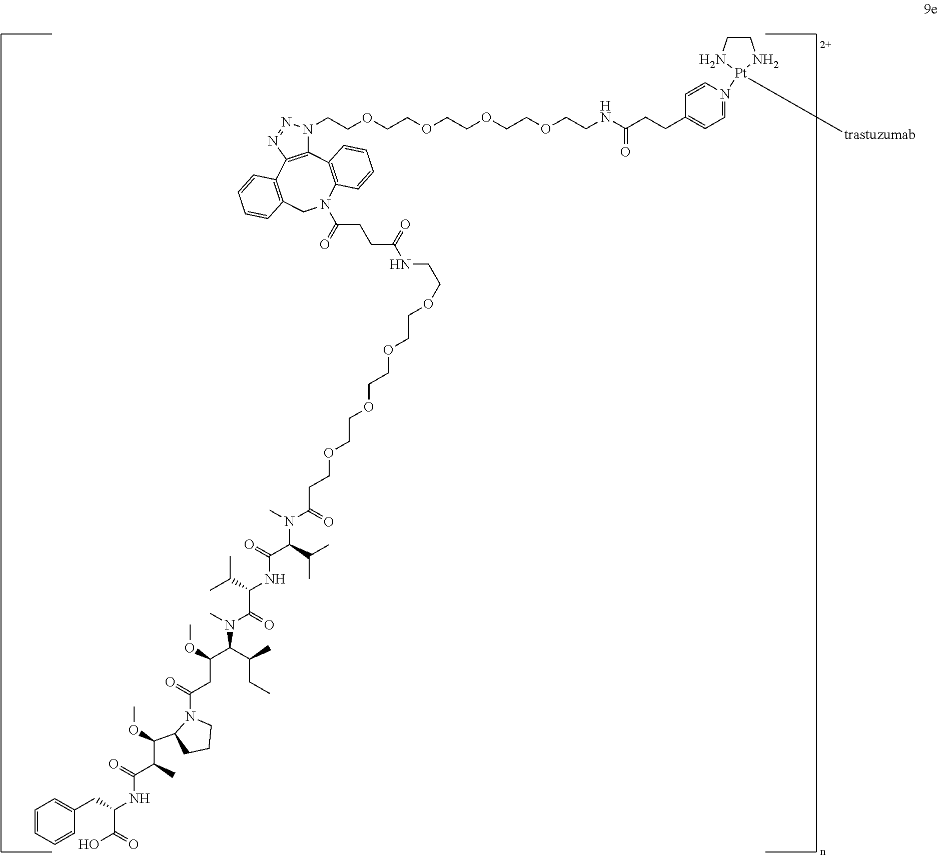

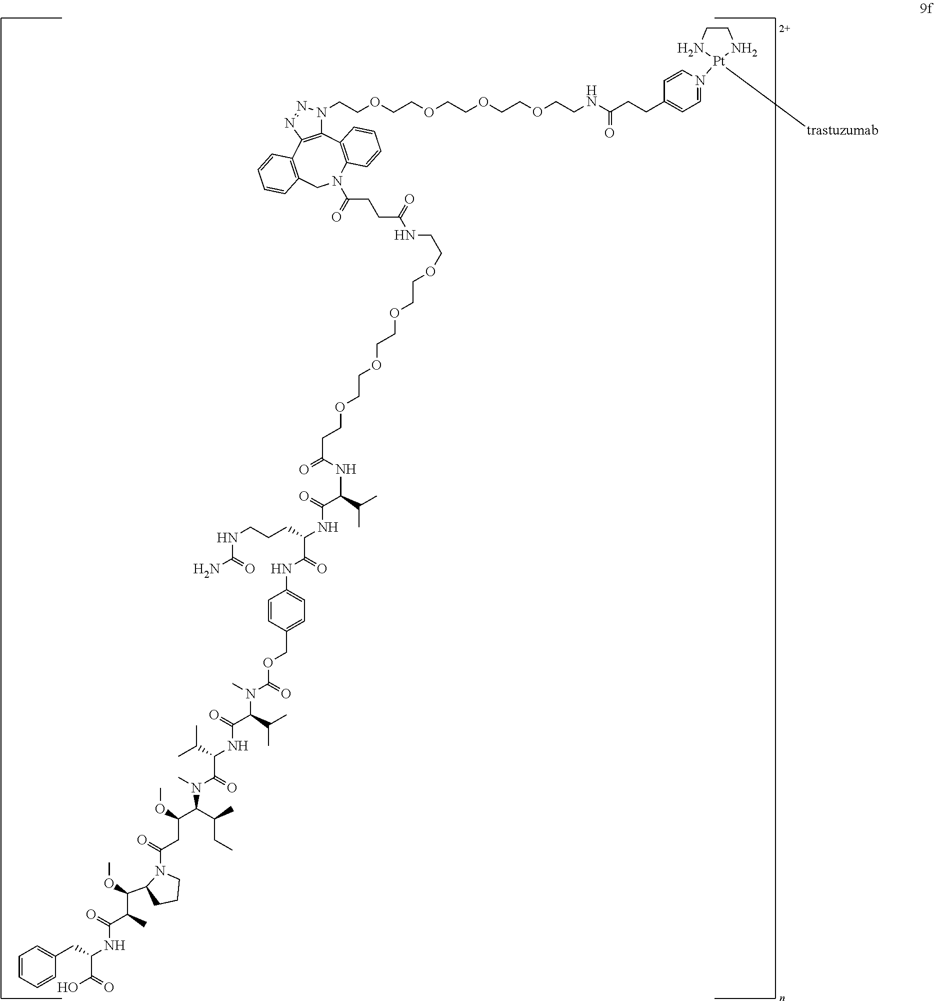

### 9.1. Synthesis of the bioconjugate trastuzumab-[Pt(Fluor 545-PEG₄-DBCO-triazole-PEG₄-pyridine)]ₙ (9a)

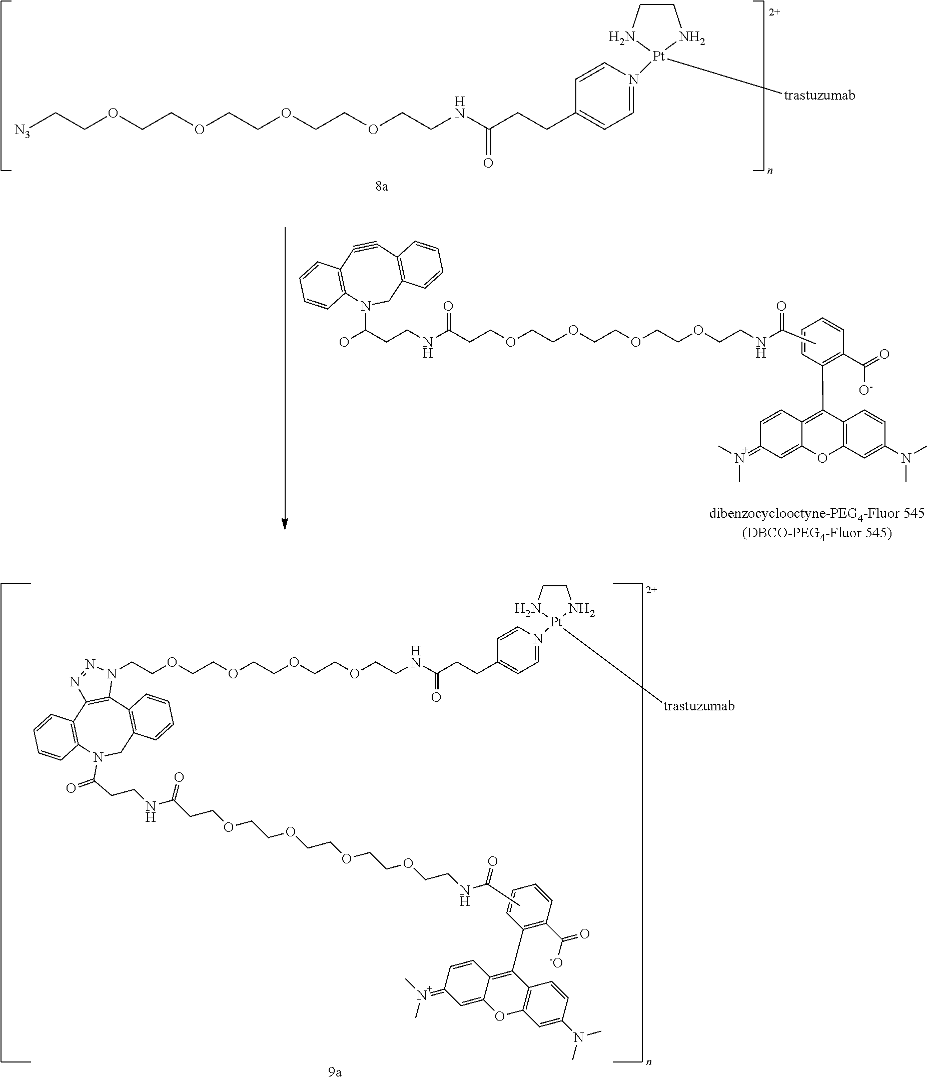

The bioconjugate 8a (303 μL, 4.95 mg/mL, 1.5 mg, 10 nmol, 1.0 eq.) was diluted with PBS (297 μL) and dibenzocyclooctyne-PEG₄-Fluor 545 (DBCO-PEG₄-Fluor 545; 10 μL, 10 mM in DMSO, 200 nmol, 20.0 eq.) was added. The sample was incubated in a thermoshaker at 37° C. for 2 h, after which the conjugate was purified by PD-10 column (equilibrated with phosphate buffered saline), followed by spin filtration using 30 kD MWCO filters (washed 1× with PBS buffer), after which it was reconstituted and stored in PBS buffer. The conjugation afforded a conjugate, which was 98.4% monomeric.

### 9.2. Synthesis of the bioconjugate trastuzumab-[Pt(BODIPY FL-DBCO-triazole-PEG₄-pyridine)]n (9b)

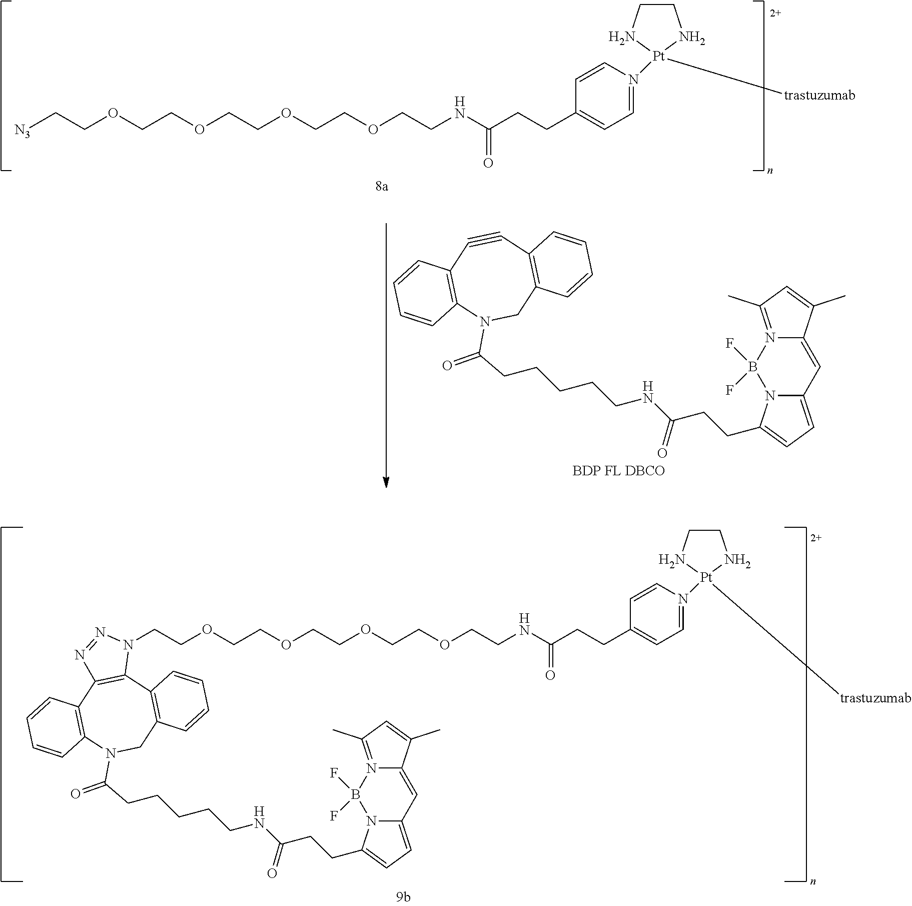

Bioconjugate 8a (57.6 μL, 4.34 mg/mL, 0.25 mg, 1.65 nmol, 1.0 eq.) was diluted with DMSO (57.6 μL) and BDP FL DBCO (2 μL, 10 mM in DMSO, 20 nmol, 12.1 eq.) was added. The sample was incubated in a thermoshaker at 37° C. for 2 h, after which the conjugate was purified by PD-10 column (equilibrated with phosphate buffered saline), followed by spin filtration using 30 kD MWCO filters (washed 1× with PBS buffer), after which it was reconstituted and stored in PBS buffer. The conjugation afforded a conjugate, which was 100% monomeric.

### 9.3. Synthesis of the bioconjugate trastuzumab-[Pt(Cyanine5 DBCO-triazole-PEG₄-pyridine)]ₙ (9c)

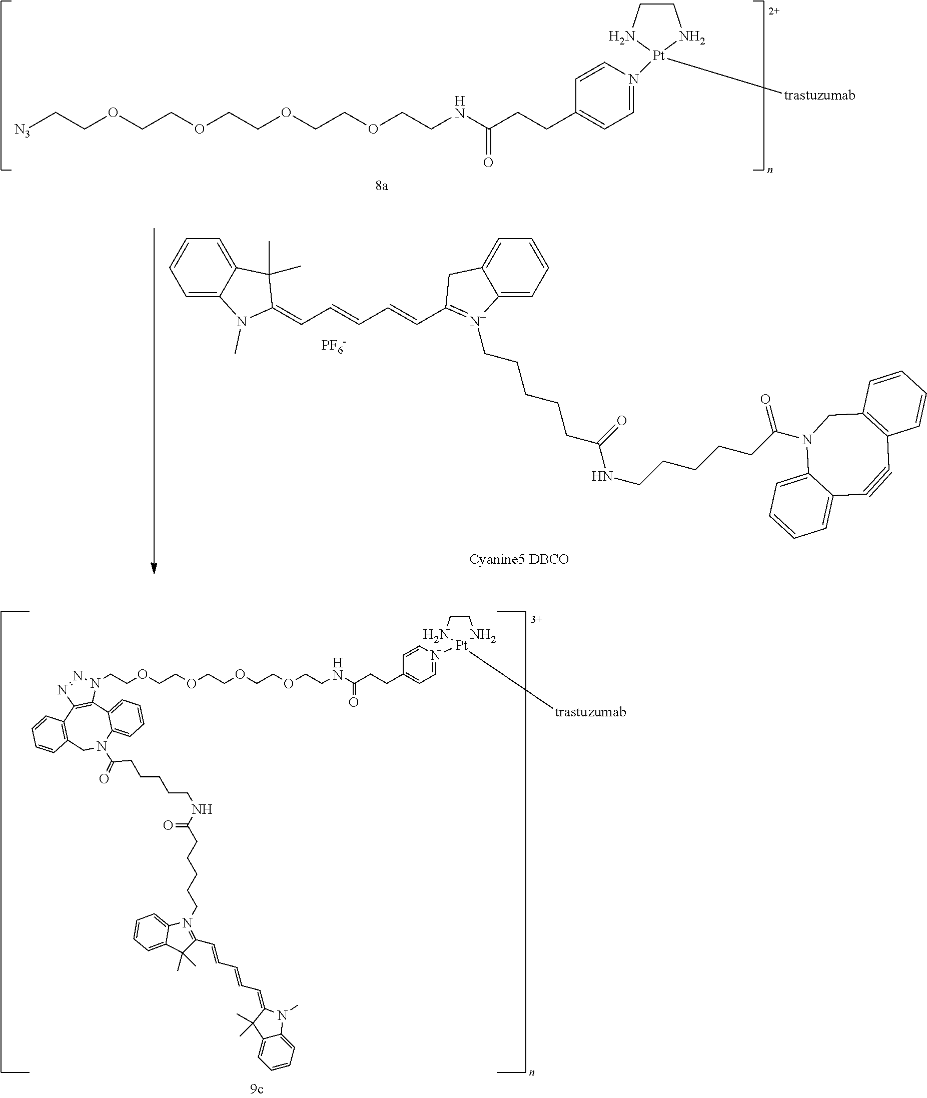

Bioconjugate 8a (57.6 μL, 4.34 mg/mL, 0.25 mg, 1.65 nmol, 1.0 eq.) was diluted with DMSO (57.6 μL) and Cyanine5 DBCO (2 μL, 10 mM in DMSO, 20 nmol, 12.1 eq.) was added. The sample was incubated in a thermoshaker at 37° C. for 2 h, after which the conjugate was purified by PD-10 column (equilibrated with phosphate buffered saline), followed by spin filtration using 30 kD MWCO filters (washed 1× with PBS buffer), after which it was reconstituted and stored in PBS buffer. The conjugation afforded a conjugate, which was 99.1% monomeric.

### 9.4. Synthesis of the bioconjugate trastuzumab-[Pt(DFO-DBCO-triazole-PEG₄-pyridine)]ₙ (9d)

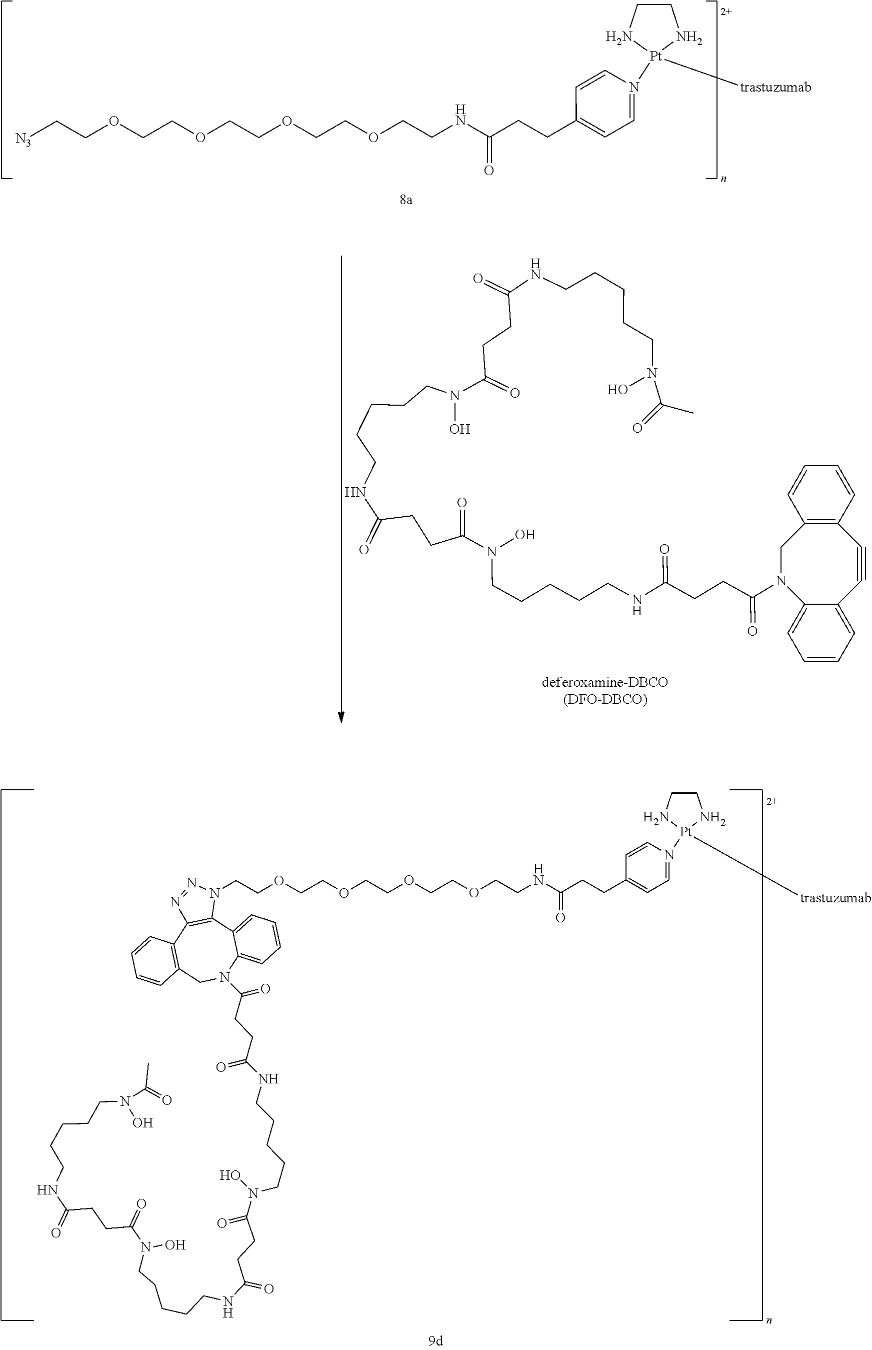

Bioconjugate 8a (300 μL, 5.0 mg/mL, 1.5 mg, 10 nmol, 1.0 eq.) was mixed with deferoxamine-DBCO (DFO-DBCO; 4 μL, 10 mM in DMSO, 40 nmol, 4.0 eq.). The sample was incubated in a thermoshaker at 25° C. for 2 h, after which the conjugate was purified by spin filtration using 30 kD MWCO filters (washed 4× with 0.9% NaCl), after which it was reconstituted and stored in 0.9% NaCl buffer. The conjugation afforded a conjugate, which was 97.8% monomeric.

### 9.5. Synthesis of the bioconjugate trastuzumab-[Pt(MMAF-PEG₄-DBCO-triazole-PEG₄-pyridine)]n (9e)

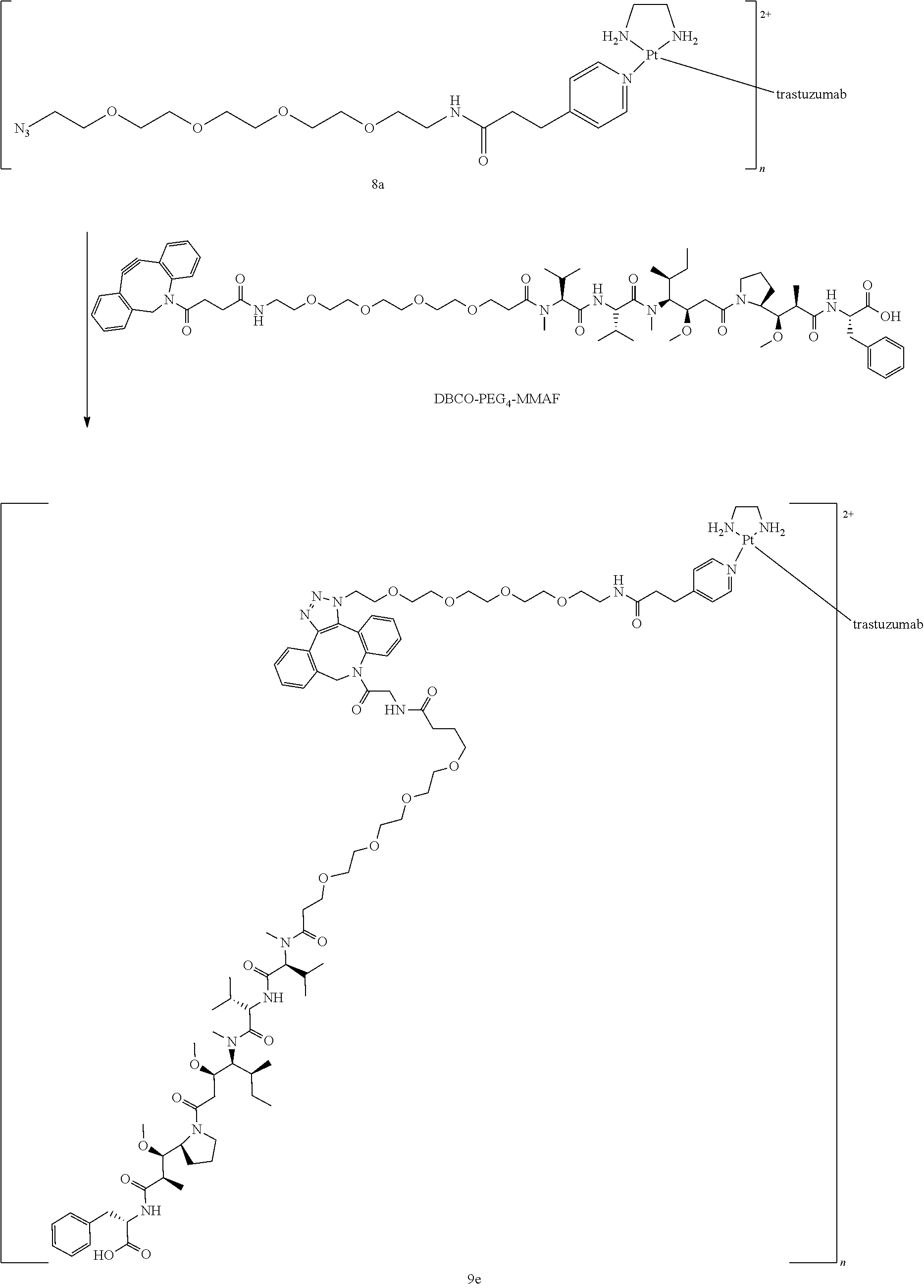

Bioconjugate 8a (300 μL, 5.0 mg/mL, 1.5 mg, 10 nmol, 1.0 eq.) was mixed with DBCO-PEG₄-MMAF (4 μL, 10 mM in DMSO, 40 nmol, 4.0 eq.). The sample was incubated in a thermoshaker at 25° C. for 2 h, after which the conjugate was purified by spin filtration using 30 kD MWCO filters (washed 4× with PBS), after which it was reconstituted and stored in PBS buffer. The conjugation afforded a conjugate, which was 97.4% monomeric and with a DAR of 2.4.

### 9.6. Synthesis of the bioconjugate trastuzumab-[Pt(MMAF-PAB-vc-PEG4-DBCO-triazole-PEG₄-pyridine)]ₙ (9f)

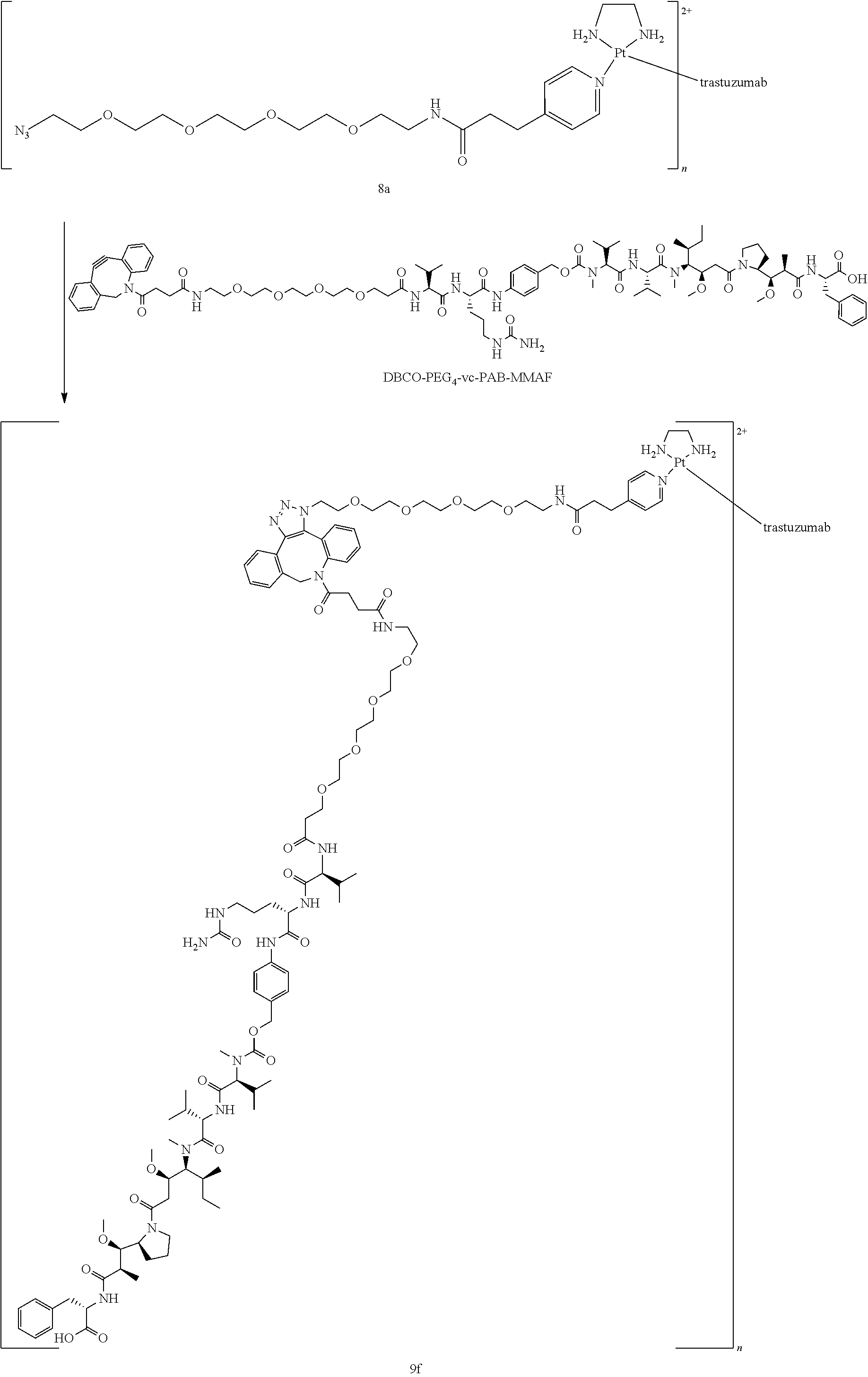

Bioconjugate 8a (300 μL, 5.0 mg/mL, 1.5 mg, 10 nmol, 1.0 eq.) was mixed with DBCO-PEG₄-vc-PAB-MMAF (4 μL, 10 mM in DMSO, 40 nmol, 4.0 eq.). The sample was incubated in a thermoshaker at 25° C. for 2 h, after which the conjugate was purified by spin filtration using 30 kD MWCO filters (washed 4× with PBS), after which it was reconstituted and stored in PBS buffer. The conjugation afforded a conjugate, which was 97.4% monomeric and with a DAR of 2.4.

### Example 10: Example of Trastuzumab-Lx Conjugate 10a Obtained from the Conjugate 8b Via the Copper-Free Click Chemistry

### 10.1. Synthesis of the bioconjugate trastuzumab-[Pt((Fluor 545-PEG₄-DBCO-triazole-PEG₄)₂-benzene-pyridine)]ₙ (10a)

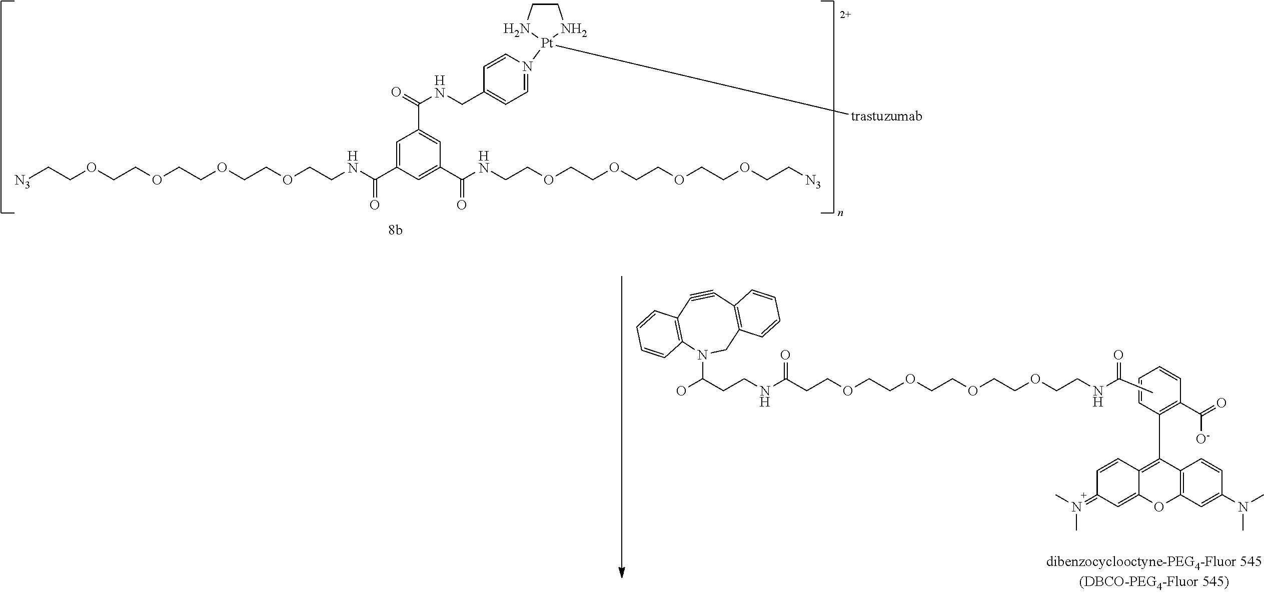

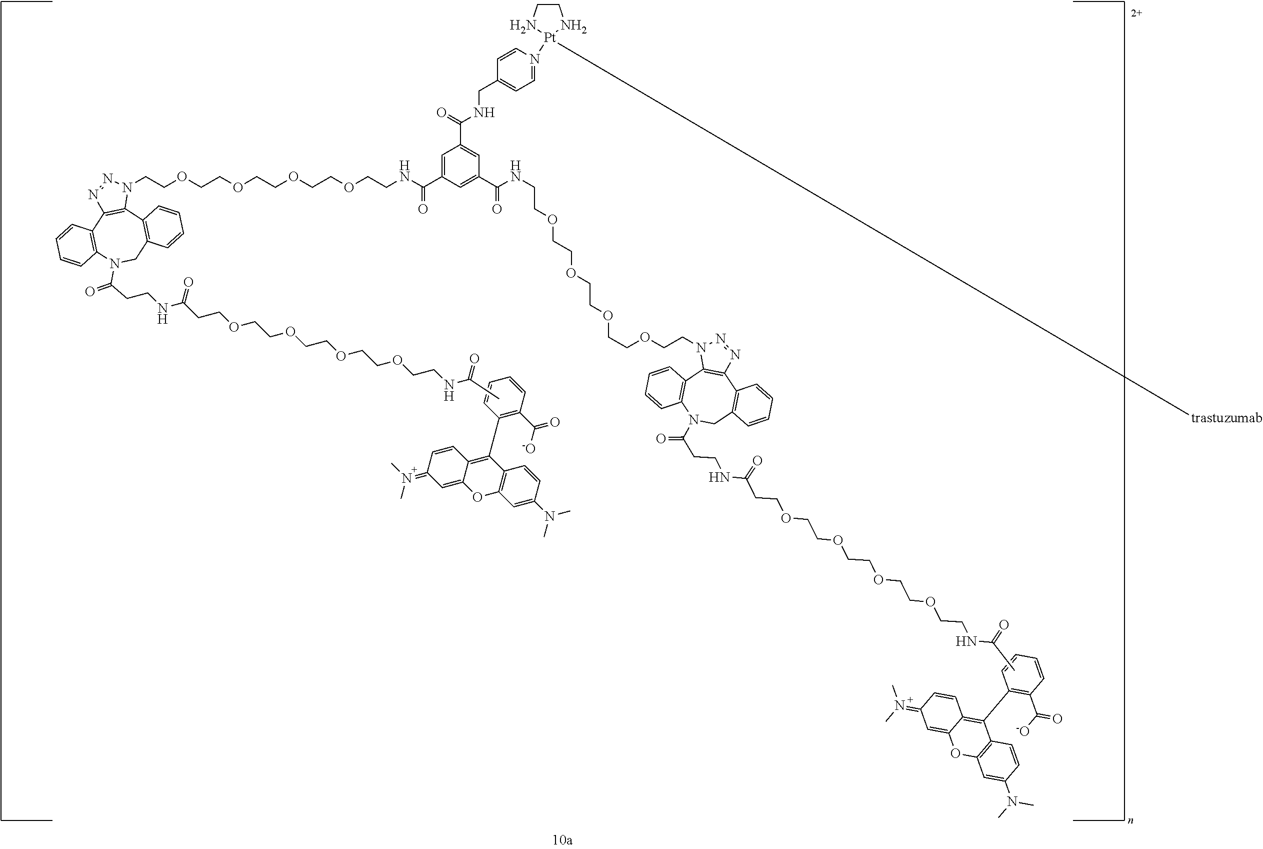

Bioconjugate 8b (303 μL, 4.95 mg/mL, 1.5 mg, 10 nmol, 1.0 eq.) was diluted with PBS (297 μL) and dibenzocyclooctyne-PEG₄-Fluor 545 (DBCO-PEG₄-Fluor 545; 20 μL, 10 mM in DMSO, 200 nmol, 20.0 eq.) was added. The sample was incubated in a thermoshaker at 37° C. for 2 h, after which the conjugate was purified by PD-10 column (equilibrated with phosphate buffered saline), followed by spin filtration using 30 kD MWCO filters (washed 1× with PBS buffer), after which it was reconstituted and stored in PBS buffer. The conjugation afforded a conjugate, which was 98.6% monomeric.

1.-34. (canceled)

35. A method for preparing a cell targeting conjugate, which conjugate comprises a cell binding moiety conjugated to a secondary functional moiety, the method comprising:
a. providing a secondary functional moiety, which secondary moiety comprises a transition metal complex having a primary functional moiety as a first ligand and iodide or bromide as a second ligand;
b. providing a cell binding moiety and binding the secondary functional moiety to the cell binding moiety via substitution of the second ligand with the cell binding moiety; and
c. treating the conjugate of step b) with a nucleophilic agent and purifying the formed cell targeting conjugate.

36. A method for preparing a cell targeting conjugate, which conjugate comprises a cell binding moiety conjugated to a functional moiety, the method comprising:
a. providing a transition metal complex comprising a first and a second leaving ligand each selected from the group consisting of iodide, bromide, and chloride;
b. providing a primary functional moiety and binding the functional moiety to the transition metal complex via substitution of the first leaving ligand by the primary functional moiety, such that a secondary functional moiety is obtained comprising the primary functional moiety as a first ligand and iodide, bromide or chloride as a second ligand;
c. mixing the secondary functional moiety of step b) with an iodide and/or a bromide releasing agent, such that the second ligand of the secondary functional moiety is iodide or bromide;
d. providing a cell binding moiety and letting the secondary functional moiety bind to the cell binding moiety via substitution of the iodide or bromide of the secondary functional moiety with the cell binding moiety; and
e. treating the cell targeting conjugate of step d) with a nucleophilic agent and purifying the formed cell targeting conjugate.

37. The method according to claim 35, wherein the transition metal complex comprises a spacer moiety able to form a bond with the primary functional moiety, such that a secondary functional moiety is formed.

38. The method according to claim 36, wherein step c) and step d) are combined in one step.

39. The method according to claim 35, wherein after the secondary functional moiety is formed, the moiety is isolated and stored for a later formation of cell targeting conjugates.

40. The method according to claim 35, wherein the transition metal complex is a platinum(II) complex.

41. The method according to claim 40, wherein the transition metal complex is a cis-platinum(II) complex or a cis-platinum(II) complex comprising a bidentate ligand.

42. The method according to claim 41, wherein the transition metal complex comprises ^{195m}Pt.

43. The method according to claim 35, wherein the secondary functional moiety is represented by the following formula: 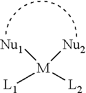
wherein M is a transition metal complex; one of the ligands L₁ or L₂ is a leaving ligand chosen from iodide, bromide or chloride, and the other ligand is a primary functional moiety; Nu is a nucleophilic group wherein Nu₁ and Nu₂ can be the same groups or different groups and which together form a bidentate ligand.

44. The method according to claim 43, wherein Nu₁ and Nu₂ together form a bidentate represented by one of the following formulas:

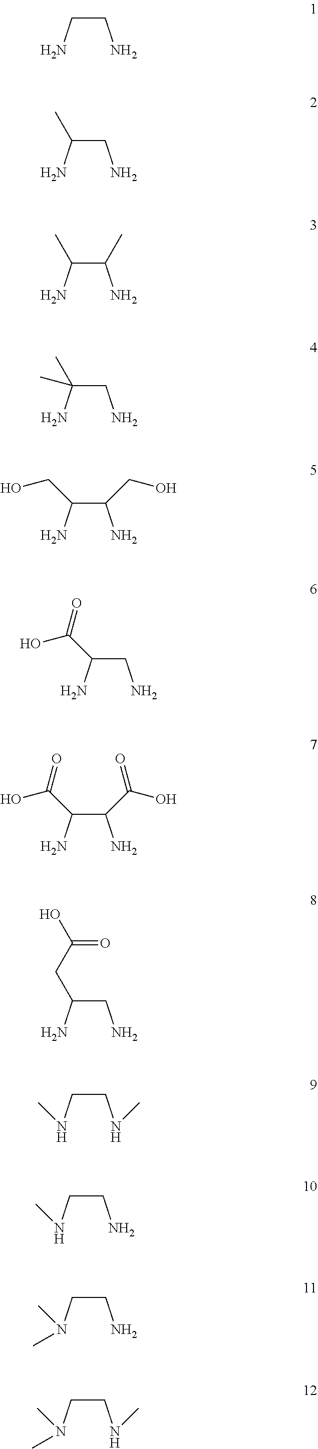

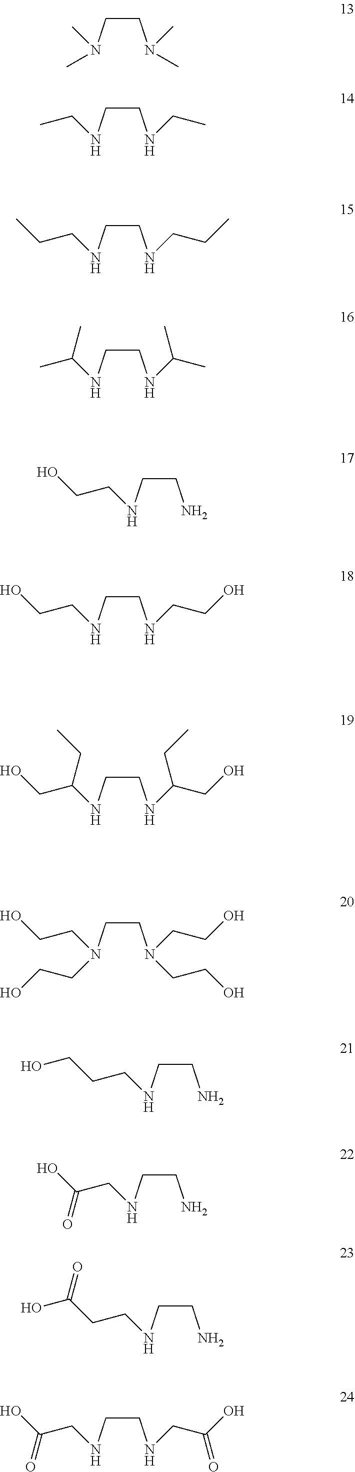

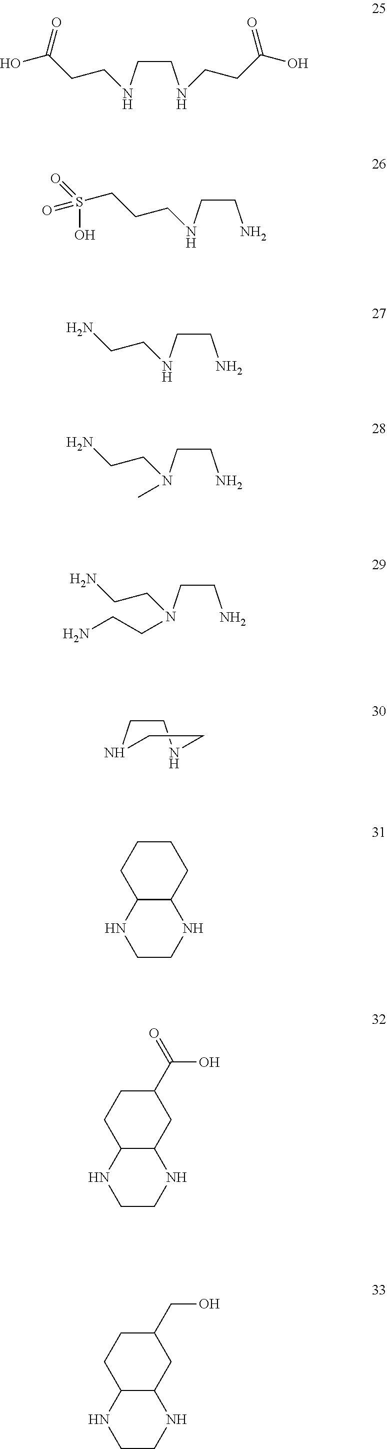

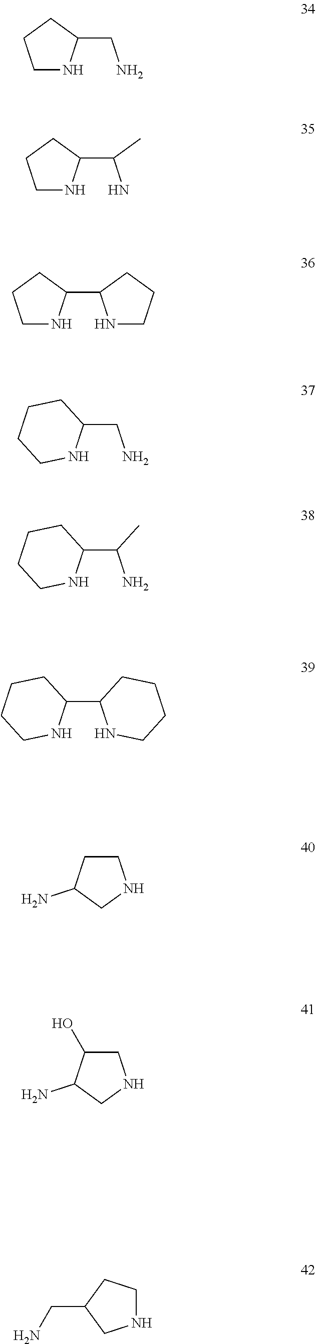

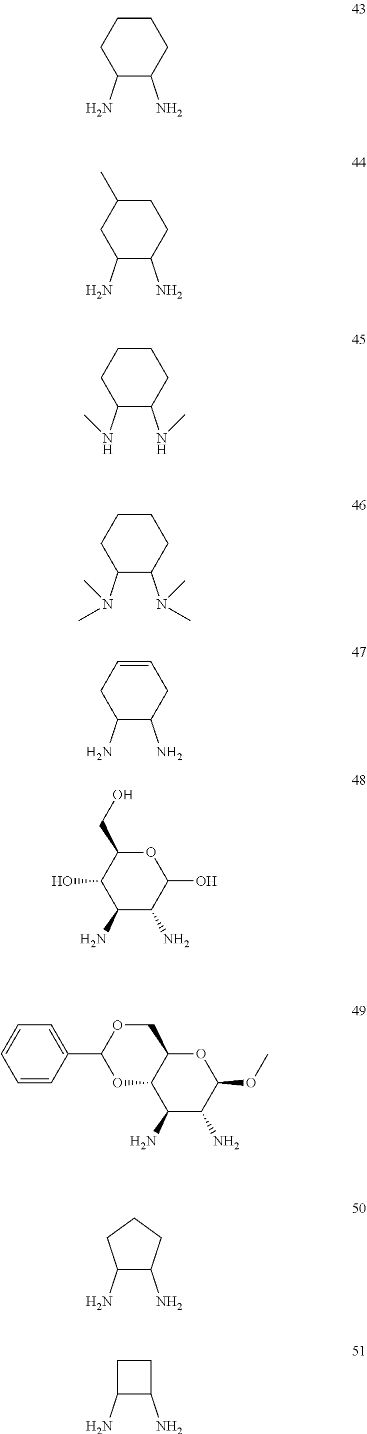

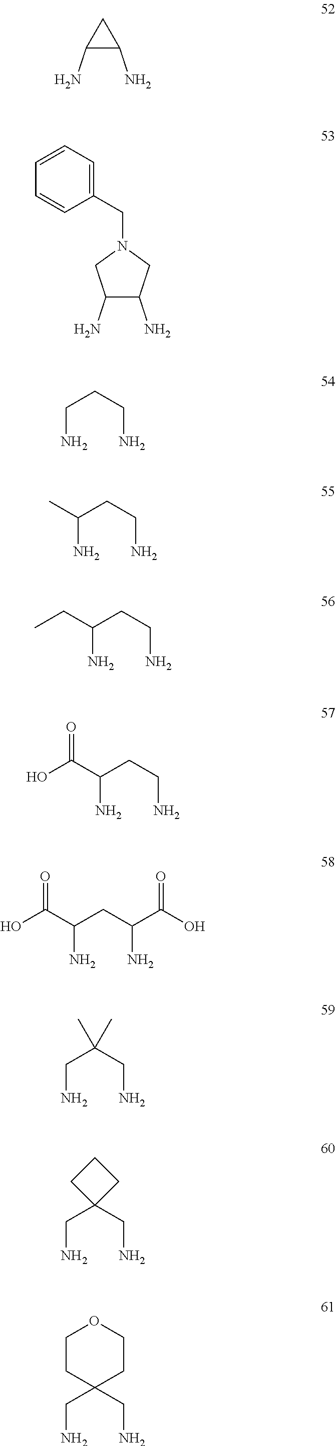

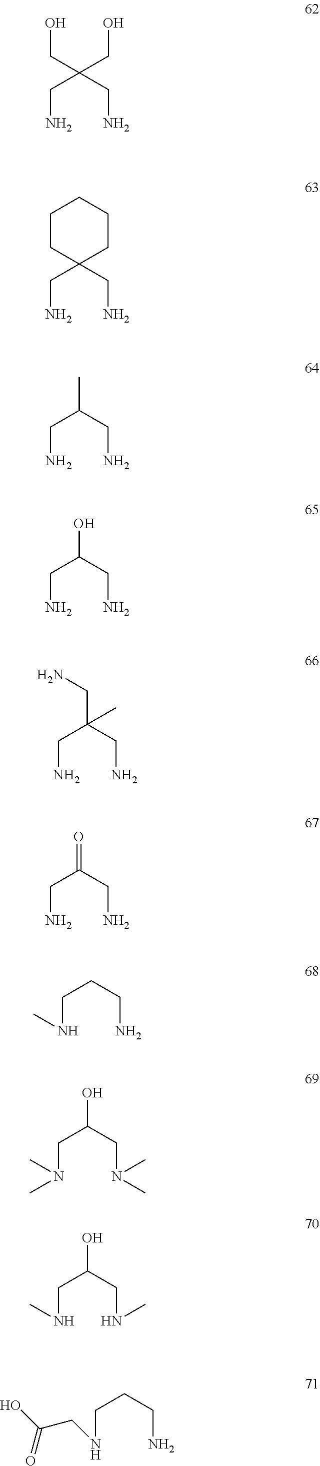

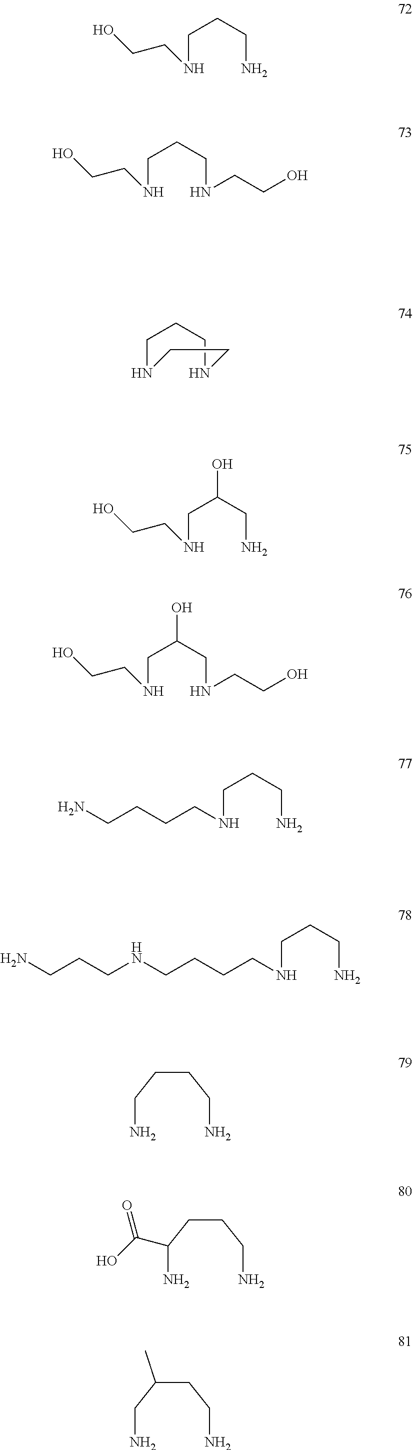

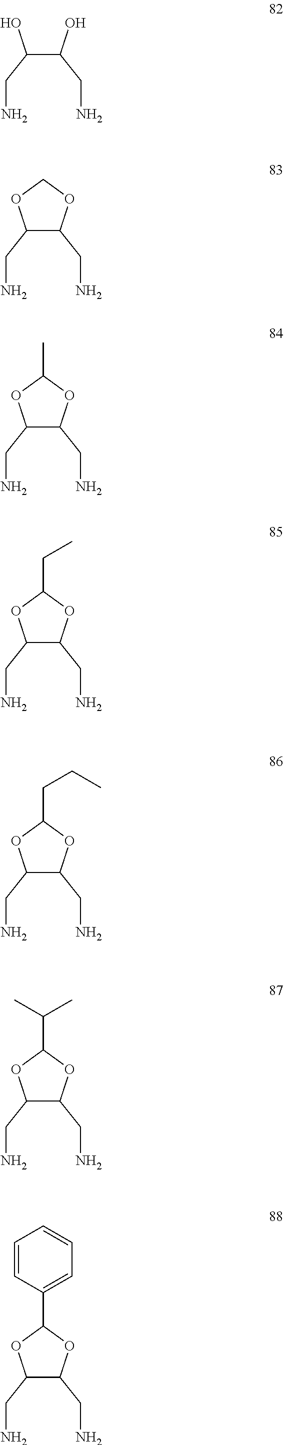

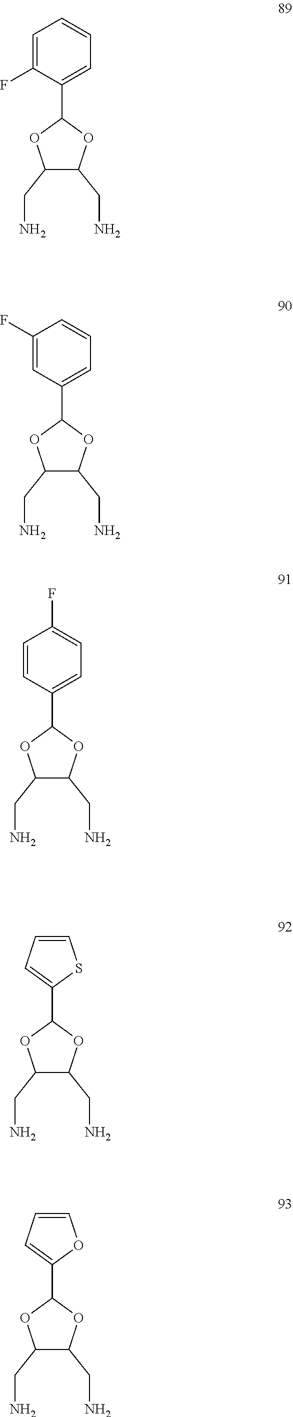 [SMILES: NCC1OC(c2ccccc2F)OC1CN ; NCC1OC(c2cccc(F)c2)OC1CN ; NCC1OC(c2ccc(F)cc2)OC1CN ; NCC1OC(c2cccs2)OC1CN ; NCC1OC(c2ccco2)OC1CN]

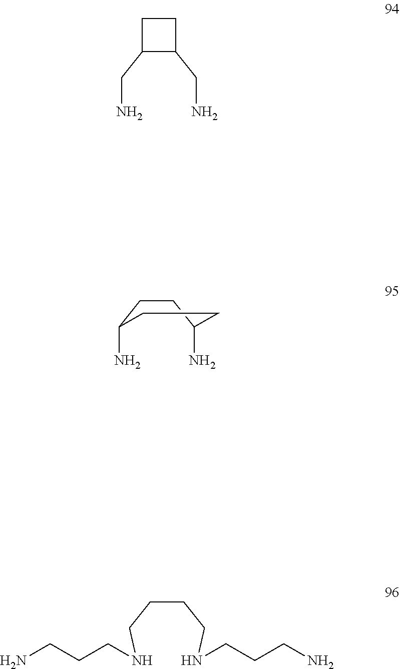 [SMILES: NCC1CCC1CN ; NC1CCC(N)CC1 ; NCCCNCCCCNCCCN]

45. The method according to claim 35, wherein the primary functional moiety is selected from the group consisting of a therapeutic compound, a diagnostic compound, a chelating agent, a dye, and a model compound.

46. The method according to claim 45, wherein the primary functional moiety is a therapeutic cytotoxic compound.

47. The method according to claim 46, wherein the cytotoxic compound is selected from the group consisting of an auristatin, a dolastatin, a symplostatin, a maytansinoid, a tubulysin, HTI-286, a calicheamycin, a duocarmycin, a pyrrolobenzodiazepine (PBD), an indolino-benzodiazepine (IGN), a camptothecin, an anthracycline, an azonafide, an amanitin, a cryptophycin, rhizoxins, epothilones, a spliceostatin, a thailanstatin, a colchicine, an aplyronine, a taxoid, methotrexate, an aminopterin, vinca alkaloids, a proteinaceous toxin, a fragment of Pseudomonas exotoxin-A, a statin, ricin A, gelonin, saporin, interleukin-2, interleukin-12, a viral protein, E4, f4, apoptin, NS1, a non-viral protein, HAMLET, TRAIL, and mda-7.

48. The method according to claim 45, wherein the primary functional moiety is a diagnostic compound selected from the group consisting of PET-imageable agents, SPECT-imageable agents, MRI-imageable agents, IRDye800CW, DY-800, ALEXA FLUOR®750, ALEXA FLUOR 790, indocyanine green, FITC, BODIPY dyes, and rhodamine dyes.

49. The method according to claim 35, wherein an iodide or bromide releasing agent is selected from the group consisting of NaI, KI, LiI, CsI, RbI, NH₄I, MgI₂, CaI₂, SrI₂, MnI₂, InI₃, AlI₃, GeI₄, guanidinium iodide, tetramethyl ammonium iodide, acetylcholine iodide, 5-(2-hydroxyethyl)-3,4-dimethylthiazolium iodide, trimethylsulfoxonium iodide, NaBr, KBr, LiBr or a mixture thereof, and a mixture thereof.

50. The method according to claim 35, having a halide concentration is for an iodide salt between 0.1 and 100 mM.

51. The method according to claim 35, wherein the pH during the binding of the secondary functional moiety to the cell binding moiety to form cell targeting conjugates ranges between 5.5 and 10.0.

52. The method according to claim 35, wherein the cell binding moiety is an antibody, a single chain antibody, an antibody fragment, a monoclonal antibody, an engineered monoclonal antibody, a single chain monoclonal antibody, monoclonal antibody of fragment thereof that specifically binds to a target cell, a chimeric antibody, a chimeric antibody fragment, or a non-traditional protein scaffold, an affibody, anticalin, adnectin, or darpin that specifically binds to a target cell.

53. The method according to claim 35, wherein the cell targeting conjugate comprises 1-10 functional moieties, each functional moiety linked to the cell binding moiety.

54. The method according to claim 35, wherein the cell binding moiety is an antibody selected from the group consisting of trastuzumab, cetuximab, rituximab, ofatumumab, and obinutuzumab.

55. The method according to claim 35, wherein a ratio between the functional moieties and the cell binding moieties is between 1:1 to 10:1.

56. The method according to claim 35, wherein the cell targeting conjugates are separated from the composition used to prepare them.

57. The method according to claim 35, wherein the secondary functional moiety used is represented by the following formula: 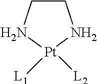
wherein one of the ligands L₁ or L₂ is a leaving ligand chosen from iodide, bromide or chloride, and the other ligand is an auristatin or derivative thereof.

58. The method according to claim 35, wherein the secondary functional moiety used is represented by the following formula: 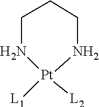
wherein one of the ligands L₁ or L₂ is a leaving ligand chosen from iodide, bromide or chloride, and the other ligand is an auristatin or derivative thereof.

59. The method according to claim 35, wherein the secondary functional moiety used is represented by the following formula: 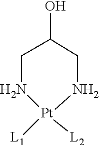
wherein one of the ligands L₁ or L₂ is a leaving ligand chosen from iodide, bromide or chloride, and the other ligand is an auristatin or derivative thereof.

60. The method according to claim 57, wherein the cell binding moiety used is trastuzumab.

61. A cell targeting conjugate and/or secondary functional moiety produced by the method according to claim 61.

62. A pharmaceutical composition comprising:
the cell targeting conjugate of claim 61, and
a pharmaceutically acceptable carrier.

63. A composition comprising:
the secondary functional moiety of claim 61 and
a pharmaceutically acceptable carrier.

64. A method of treating cancer in a mammal, the method comprising:
utilizing the cell targeting conjugate of claim 61 to treat the mammal.

65. The method according to claim 64, wherein the cancer is selected from the group consisting of colorectal cancer, breast cancer, pancreatic cancer, and non-small cell lung carcinomas.

66. The method according to claim 65, wherein the cancer is breast cancer having a low expression level of Her2.

67. A secondary functional moiety produced by the method according to claim 36.

68. A secondary functional moiety comprising:
a transition metal complex having a primary functional moiety as a first ligand and iodide or bromide as a second ligand.
